SEQUENCE LISTING (11 sequences)

SEQ ID NO: 1 - protein, 27 aa, synthetic construct
  MOD_RES 19..27: note = D-Arg
WEADIVADIVADIVAGGGRRRRRRRRR

SEQ ID NO: 2 - protein, 31 aa, synthetic construct
  MOD_RES 23..31: note = D-Arg
WEADIVADIVHDIVADIVAGGGRRRRRRRRR

SEQ ID NO: 3 - protein, 31 aa, synthetic construct
  MOD_RES 23..31: note = D-Arg
WEADIVADIVWDIVADIVAGGGRRRRRRRRR

SEQ ID NO: 4 - protein, 9 aa, synthetic construct
  MOD_RES 1: note = pyroglycine
  MOD_RES 6: note = D-Leu
GHWSYLLRP

SEQ ID NO: 5 - protein, 45 aa, synthetic construct
  MOD_RES 1: note = pyroglycine
  MOD_RES 6: note = D-Leu
  MOD_RES 37..45: note = D-Arg
GHWSYLLRPGGGGGWEADIVADIVWDIVADIVAGGGRRRRRRRRR

SEQ ID NO: 6 - protein, 31 aa, synthetic construct
  MOD_RES 23..31: note = D-Arg
  VARIANT 11: note = Xaa may be Ala, His or Trp
WEADIVADIVXDIVADIVAGGGRRRRRRRRR

SEQ ID NO: 7 - protein, 4 aa, synthetic construct
GFLG

SEQ ID NO: 8 - RNA, 21 nt, synthetic construct
  source 1..21: mol_type = other RNA
cacgtttgagtccatgcccaa

SEQ ID NO: 9 - RNA, 19 nt, synthetic construct
  source 1..19: mol_type = other RNA
atggagacccgctgaacaa

SEQ ID NO: 10 - protein, 45 aa, synthetic construct
  MOD_RES 1: note = pyroglycine
  MOD_RES 6: note = D-Leu
  MOD_RES 37..45: note = D-Arg
GHWSYLLRPGGGVAWEADIVADIVWDIVADIVAGGGRRRRRRRRR

SEQ ID NO: 11 - protein, 47 aa, synthetic construct
  MOD_RES 1: note = pyroglycine
  MOD_RES 6: note = D-Leu
  MOD_RES 39..47: note = D-Arg
GHWSYLLRPGGGGFLGWEADIVADIVWDIVADIVAGGGRRRRRRRRR

(19) United States
(12) Patent Application Publication
(10) Pub. No.: US 2025/0177548 A1
(43) Pub. Date: Jun. 5, 2025

(54) FUSOGENIC PEPTIDE COMPOSITIONS AND METHODS OF CARGO DELIVERY

(71) Applicant: CLEMSON UNIVERSITY, Clemson, SC (US)

(72) Inventors: Timothy SAMEC, Tampa, FL (US); Angela ALEXANDER-BRYANT, Easley, SC (US); K. Lora ALATISE, Central, SC (US)

(21) Appl. No.: 18/833,661

(22) PCT Filed: Jan. 27, 2023
(86) PCT No.: PCT/US2023/061436

§ 371 Date: Jul. 26, 2024

Related U.S. Application Data
(60) Provisional application No. 63/303,670, filed on Jan. 27, 2022.

(51) Int. Cl. A61K 47/68; A61K 47/64; C07K 14/00

(52) U.S. Cl. CPC A61K 47/6807; A61K 47/64; C07K 14/001; C07K 2319/33; C07K 2319/85

(57) ABSTRACT

The present invention is based on the identification of fusogenic polypeptides that not only enhance cellular uptake, but also enhance the fusogenicity of the polypeptides to cause endosomal membrane destabilization, resulting in permeabilization and release of a cargo into the cytosol. Thus, one aspect of the invention relates to a fusogenic polypeptides having a peptide sequence with amphipathic properties and a peptide sequence having multiple positively charged amino acid residues for the binding of a cargo. A further aspect of the invention relates to methods using the fusogenic polypeptides for delivery of cargo into a cell.

### STATEMENT OF PRIORITY

This application claims the benefit, under 35 U.S.C. § 119 (e), of U.S. Provisional Application Ser. No. 63/303,670, filed Jan. 27, 2022, the entire contents of which are incorporated by reference herein.

### STATEMENT OF GOVERNMENT SUPPORT

This invention was made with government support under Grant Nos. OIA-1655740 and 204669 awarded by the National Science Foundation. The government has certain rights in the invention.

### STATEMENT REGARDING ELECTRONIC FILING OF A SEQUENCE LISTING

A Sequence Listing in XML format, entitled 9662-76WO_ST26.xml, 16,542 bytes in size, generated on Jan. 27, 2023 and filed herewith, is hereby incorporated by reference in its entirety for its disclosures.

### FIELD OF THE INVENTION

The invention relates to fusogenic peptides and the use thereof for delivery of cargo to a cell.

### BACKGROUND OF THE INVENTION

Epithelial ovarian cancer is the 7th most commonly diagnosed cancer among women in the United States and has the highest mortality among all gynecological cancers. Ovarian cancer is commonly diagnosed in late stages with low response to therapeutic interventions with many patients experiencing significant systemic toxicity and drug-resistant metastatic cancer and in the past 10 years, advanced-stage patients have only had an increase in survival rates of 1%. Because over 90% of ovarian cancers are derived from epithelial cells, tumors are prone to rapid proliferation and spreading, causing both local and distant metastases within ascites fluid. Approximately 80% of patients will experience recurrence, multidrug resistance, or distant metastasis when treated for advanced-stage disease. To improve patient outcomes, there is a need for more effective ovarian cancer treatment strategies delivered via robust, biocompatible, and reliable mechanisms.

Gene therapy through RNA interference (RNAi) has been extensively studied as a potential therapeutic intervention in many cancer types since its discovery in 1998. RNAi enables gene silencing through sequence-specific inhibition of messenger RNA (mRNA) translation using short hairpin RNA (shRNA) or short interfering RNA (siRNA). RNAi technology has significant implications in cancer treatment through the silencing of proteins responsible for cancer cell survival, migration, invasion, regrowth, and development of multidrug resistance. Although this mechanism is highly specific and has been shown to be advantageous in drug resistant cancers, siRNA delivery can be difficult to achieve without a nanocarrier due to its size, negative charge, and rapid degradation within the endosome.

Peptide-based systems have shown great ability to complex with and efficiently deliver siRNA for sequence-specific mRNA degradation in cancer, providing a method of eluding barriers to siRNA delivery, including rapid RNase-induced degradation, inefficient cell uptake, and lack of endosomal escape. More specifically, fusogenic peptide sequences efficiently complex siRNA through electrostatic interactions, are taken up via endocytosis, and can cause endosomal escape via membrane disruption. Due to their amphipathic nature, fusogenic peptides interact with the cell membrane via hydrophobic amino acid residue affinity for the lipid bilayer membrane, inducing endocytosis. When encapsulated in the endosome, fusogenic peptides undergo a pH-dependent conformational change, primarily forming an α-helical structure, though some β-sheet formation has been recorded and reported to have similar fusion with the endosomal membrane. This newly conformed peptide structure can then insert itself into the endosomal membrane via the projection of hydrophobic amino acid residues, resulting in membrane structure destabilization and breakdown, allowing for the internalized cargo to dissociate from the fusogenic peptide delivery system and escape into the cytosol (FIG. 1).

There is a need in the art for improved fusogenic peptides that enhance intracellular delivery, endosomal escape, and therapeutic effectiveness.

### SUMMARY OF THE INVENTION

The present invention is based on the identification of fusogenic peptides that not only enhance cellular uptake, but also enhance the fusogenicity of the peptides to cause endosomal membrane destabilization, resulting in permeabilization and release of a cargo into the cytosol. Thus, one aspect of the invention relates to a fusogenic peptide for delivery of a cargo to a cell comprising a first peptide sequence with amphipathic properties and a second peptide sequence comprising one or more positively-charged amino acid residues for binding the cargo.

Another aspect of the invention relates to a composition, e.g., a pharmaceutical composition, comprising one or more of the fusogenic peptides and cargo of the invention. The fusogenic peptide and cargo may be in the form of a nanoparticle complex.

A further aspect of the invention relates to a method of delivering cargo into a cell, comprising contacting the cell with the fusogenic peptide and cargo of the invention, thereby delivering the cargo to the cell.

An additional aspect of the invention relates to a method of inhibiting expression of a gene in a cell, comprising contacting the cell with the fusogenic peptide and cargo (e.g., siRNA that targets the gene) of the invention, thereby inhibiting expression of the gene in the cell.

Another aspect of the invention relates to a method of treating, preventing, or delaying progression of a disease or condition (e.g., cancer) in a subject in need thereof, comprising administering to the subject an effective amount of the pharmaceutical composition of the fusogenic peptide and cargo of the invention, thereby treating, preventing, or delaying progression of a disease or condition.

Another aspect of the invention relates to a method of inhibiting expression of a human CK2α protein in a cell, comprising contacting the cell with the fusogenic peptide and cargo of the invention, wherein the cargo is an siRNA targeting the CSNK2A1 gene, thereby inhibiting expression of the human CK2α protein in the cell.

An additional aspect of the invention relates to a method of treating cancer in a subject in need thereof, wherein the cancer comprises over-expression of the human CK2α protein, comprising delivering to the subject the fusogenic peptide and cargo of the invention, wherein the cargo is an siRNA targeting the CSNK2A1 gene, thereby treating cancer in the subject.

These and other aspects of the invention are set forth in more detail in the description of the invention below.

### BRIEF DESCRIPTION OF THE DRAWINGS

FIG. 1 is a pathway of fusogenic peptide nanoparticle delivery and endosomal membrane fusion. FIG. 1 shows that fusogenic peptides may address two primary barriers to siRNA therapeutic delivery in cancer through encouraging endocytosis and facilitating endosomal escape via pH-dependent protonation, conformational shift of the peptide, and insertion into the endosomal membrane. Upon membrane puncture, therapeutic cargo can dissociate from the peptide and escape into the cytosol for RNA-induced silencing complex formation and mRNA degradation.

FIGS. 2A-2F show the characterization of fusogenic peptides. Peptides were electrostatically complexed with siRNAs at increasing N:P ratios and immediately subjected to agarose gel electrophoresis (FIG. 2A) or following incubation in 50% fetal bovine serum (FBS) (FIG. 2B) or RNase A (FIG. 2C) for 1 hour. Size distribution (FIG. 2D), polydispersity index (PDI), and surface ζ-potential (FIG. 2E) were determined by dynamic light scattering and Doppler voltage velocities for each DIV3 (X) peptide/siRNA complex at a 60:1 ratio. Data are mean±standard deviation, performed in triplicate. Transmission electron microscopy confirmed the size and morphology of DIV3W-siRNA complexes at a 60:1 ratio (FIG. 2F). Scale bar: 200 nm.

FIGS. 3A-3B show the fusogenic peptide biocompatibility via MTS assay. MTS analysis indicates cell viability 48-hours after treatment with each peptide formulation using OVCAR3 (FIG. 3A) and CAOV3 (FIG. 3B) cells. Data are mean #standard error of the mean (SEM) of three independent experiments, performed in triplicate.

FIGS. 4A-4C show the fusogenic peptide uptake efficiency in vitro in OVCAR3 cells. (FIG. 4A) Fluorescence microscopy of siRNA uptake in OVCAR3 cells left untreated (UNTD), treated with siNT alone, or DIV3 (X) peptide-siNT-DY547 (red) complexes for 4 hours. Cells are counterstained with Hoechst (blue). Scale bar: 100 μm. FIGS. 4B and 4C show flow cytometric analysis of DIV3 (X)-siNT-DY547 uptake into OVCAR3 cells after 4 hours. Data are mean±SEM of three independent experiments analyzed with one-way ANOVA and post-hoc Tukey multiple comparisons testing, where * p≤0.05, ** p≤0.01, *** p≤0.001, **** p≤0.0001.

FIGS. 5A-5B show the endosomal escape ability of DIV3W. Immunofluorescence microscopy depicts early endosome antigen-1 (green) after incubation of OVCAR3 (FIG. 5A) and CAOV3 (FIG. 5B) cells with siNT-DY547 (red) complexed with DIV3 (X) peptides at a 60:1 N:P ratio for 4 and 8 hours. Colocalized green and red fluorescence (yellow) indicates endosomal entrapment of the peptide/siRNA complexes, identified with white arrows, while siRNA that has escaped can be seen in red in the overlay image. Cell nuclei are counterstained with Hoechst 33342 (blue). Scale: 20 μm.

FIGS. 6A-6D show the fusogenic-mediated bioactivity of siRNAs in OVCAR3 cells. FIGS. 6A-6C are bar graphs showing the qPCR analysis of CSNK2A1 mRNA after treatment of OVCAR3 cells with DIVA3 (FIG. 6A), DIV3H (FIG. 6B), and DIV3W (FIG. 6C) peptides complexed with siNT or siCSNK2A1 for 48 hours. Data are mean±SEM of three independent experiments analyzed with Student's t-test within each N:P ratio, where * p≤0.05 and ** p≤0.01. FIG. 6D is a Western blot showing the CSNK2A1 protein product (CK2a) after treatment of OVCAR3 cells with DIV3W-siCSNK2A1 for 48 hours. β-Actin expression was monitored to ensure equal protein loading.

FIGS. 7A-7E show the downstream cellular effects of CSNK2A1 knockdown. DIV3W, complexed with (FIG. 7A) siNT or siCSNK2A1 at 40:1, 60:1, and 80:1 N:P ratios, was delivered to OVCAR3 cells for 24 hours and the ability of the cells to migrate across a scratch wound was observed using phase microscopy imaging over 72 hours. Scale bar: 1000 μm. FIG. 7B is a graph showing cell regrowth over the course of 72 hours, which was quantified with ImageJ and represented as percent wound heal relative to the initial scratch distance. FIGS. 7C and 7D are white light imaging of clonogenic colony-forming assay of OVCAR3 (FIG. 7C) and CAOV3 (FIG. 7D) cells 14 days after treatment with DIV3W-siCSNK2A1 at 80:1 N:P ratio. FIG. 7E is a bar graph of a clonogenic recolonization assay showing colonization was quantified via plate absorbance at 490 nm and normalized to siNT negative control groups. Data are mean±SEM of three independent experiments and were analyzed using a two-way ANOVA with Tukey's multiple comparisons posthoc test and one-tailed T-test for scratch and clonogenic results, respectively, where * p≤0.05, ** p≤0.01, and **** p<0.0001.

FIGS. 8A-8F show the LHRH-DIV3W peptide sequences and their characteristics. FIG. 8A is a schematic representation of the peptide indicating general properties of various regions. FIG. 8B shows peptide/siRNA complexation at increasing N:P ratios through electrophoretic shift assay with unbound siRNA shown as white banding. FIG. 8C shows the size distribution of the three peptides DIV3W, LHRH, and LHRHDIV3W measured via dynamic light scattering (DLS). FIG. 8D is a gel shift assay of tandem peptide/siRNA complexes incubated with (+) or without (−) 50% serum or RNase A. Intact siRNA is shown with white banding. FIG. 8E shows the peptide diameter measured and quantified via DLS including zeta potential and PDI. FIG. 8F shows LHRH-DIV3W cytotoxicity analysis in OVCAR3 and CAOV3 cells. Experiments were completed to N=3 and data was analyzed using a two-way ANOVA with multiple comparisons. * p≤0.05.

FIGS. 9A-9B show the immunofluorescence microscopy of LHRH receptor (green) in OVCAR3 and CAOV3 cells (FIG. 9A) with nuclei stained with Hoescht 33342. FIG. 9B shows supporting western blot analysis of LHRH receptor protein expression in HOSEpiC lysate, OVCAR3, and CAOV3 cells. Scale: 100 μm.

FIGS. 10A-10F show the cellular uptake of siRNAs via active targeting mechanisms using the LHRH receptor targeting peptide and the DIV3W fusogenic sequence. 4 hr uptake imaging of OVCAR3 (FIG. 10A) and CAOV3 (FIG. 10B) cells incubated with LHRH-DIV3W-siNT-Cy5.5 complexes at increasing N:P ratios. siNT-Cy5.5 is stained red and nuclei are stained using Hoechst 33342 and appear blue. Uptake was quantified with flow cytometry at 4 hr (FIG. 10C) and 24 hr (FIG. 10D). LHRH receptor targeting peptide uptake efficiency compared to LHRH-DIV3W tandem peptide uptake efficiency was analyzed by flow cytometry at 4 hr (FIG. 10E). OVCAR3 cell uptake imaging comparison between LHRH receptor bound with antibody (green) prior to complex delivery, LHRH-siNT-Cy5.5, and LHRH-DIV3W-siNT-Cy5.5 (FIG. 10F). All data are mean±SEM of three independent experiments analyzed via two-way ANOVA (FIG. 10C, FIG. 10D) and multiple t-tests (FIG. 10E), where * p<0.05 compared to CAOV3 80:1 at 4 hours (FIG. 10C) and * p≤0.05, ** p≤0.01 compared to LHRH-mediated uptake. Scale (FIG. 10A, FIG. 10B): 200 μm, (FIG. 10F) 50 μm.

FIGS. 11A-11B show the tandem-mediated bioactivity of siRNAs in OVCAR3 cells. FIG. 11A is a bar graph showing the qPCR analysis of CSNK2A1 mRNA after treatment of OVCAR3 cells with LHRH-DIV3W peptide complexed with siNT or siCSNK2A1 for 48 hours. Data are mean±SEM of three independent experiments analyzed with Student's t-test within each N:P ratio, where * p<0.05. FIG. 11B is a western blot of CSNK2A1 protein product, CK2a, after treatment of OVCAR3 cells with LHRH-DIV3W-siCSNK2A1 for 48 hours. β-Actin expression was also examined to confirm equivalent protein loading.

FIGS. 12A-12B show the downstream knockdown of CSNK2A1 colony reformation analysis at multiple N:P ratios. LHRH-DIV3W-siNT and -siCSNK2A1 treated OVCAR3 cells (FIG. 12A) were harvested after treatment, reseeded, and allowed to regrow over a 2-week period. Relative colonization percent±SEM was quantified via absorbance spectroscopy at 490 nm (FIG. 12B). Experiments were completed to N=3 and data analyzed with two-way ANOVA. * p<0.05, ** p≤0.01, *** p<0.001 compared to LHRH-DIV3W-siNT treatment.

FIGS. 13A-13C show the gel shift analysis of LHRH receptor complexation with siRNAs (FIG. 13A) and protection from RNase A and 50% serum degradation (FIG. 13B). White bands indicate intact, unbound siRNAs (FIG. 13A) and siRNAs released from complexes protected from degradation (FIG. 13B). Viability of OVCAR3 and CAOV3 cells treated with LHRH peptide alone exhibiting biocompatibility are shown in FIG. 13C.

FIG. 14 shows CAOV3 cells incubated with LHRH receptor antibody prior to treatment with LHRH-DIV3W-siNT-Cy5.5. LHRH receptor competitively bound by antibody and Alexa Fluor 488 (green) in each cell line was also incubated with LHRH-DIV3W-siNT-Cy5.5 (red) fluorescence. Cells were also incubated with LHRH-DIV3W-siNT-Cy5.5 without the presence of LHRH receptor antibody.

FIGS. 15A-15B show the size distribution of the DIVA3W, DIVA3, and DIVA3H peptides (FIG. 15A) including the PDI and zeta potential of the assembled Cas9/gRNA nanocomplexes (FIG. 15B).

FIG. 16 shows peptide nanocomplexes delivered Cy3-siRNA in MCF10A cells. Fluorescence microscopy analysis with various peptides complexed with Cas9/Cy-3 labeled siRNA (red) incubated with MCF10A clover cells (green) for 4 hours. Nuclei are counterstained with DAPI (blue).

FIG. 17 shows that truncated DIVA3, DIV3H, and DIV3W peptides containing a penta-arginine tail did not fully complex with siRNAs at various N:P molar ratios. Shown as white bands, free siRNA was observed for all N:P conditions for each of the three peptide systems. MWM-molecular weight marker.

FIG. 18 is a graphical summary of in vivo experimental procedures using DIV3W peptide to deliver siRNAs in a xenograft tumor model of ovarian cancer.

FIGS. 19A-19E are heat map images and a bar graph showing results of treatment with nanocomplexes. Mice with SQ tumors (FIG. 19A) and IP tumors (FIG. 19B) were treated with either siNT-Cy5.5 or DIV3W-siNT-Cy5.5. SQ tumors and IP tumors received intratumoral and intraperitoneal injection of siRNA, respectively. Images were acquired at 3, 6, 12, and 24 hours prior to ex vivo imaging of SQ (FIG. 19C) and IP (FIG. 19D) tumors and organs, with image normalization to siNT-Cy5.5 mice using Aura software for quantitative analysis (FIG. 19E). siNT-Cy5.5 fluorescence intensity is indicated through redshift using an inverted rainbow heatmap. Quantification of siRNA biodistribution is reported as total photon emission (photons/s)=SEM of three independent samples, where **** p≤0.0001, * p≤0.05 as determined by two-way ANOVA analysis with multiple comparisons.

FIGS. 20A-20B are a graphical summary of in vivo experimental procedures and a bar graph showing the relative expression of CSNK2A1 is reduced when treated with the DIV3W-siCSNK2A1 nanocomplex. Mice treated subcutaneously (FIG. 20A) with DIV3W-siCSNK2A1 have significantly reduced CSNK2A1 mRNA expression in comparison to DIV3W-siNT and saline control treatments (FIG. 20B). Data are reported as mean #SEM of relative CSNK2A1 expression normalized to siNT for four independent experiments for siNT and siCSNK2A1, and two experiments for saline-treated groups, where * p≤0.05 and ** p≤0.01 compared to saline and DIV3W-siNT determined by one-way ANOVA analysis.

FIGS. 21A-21B are photographs showing internal metastasis of OVCAR3 cells in mice with IP tumors indicated with red circles.

FIGS. 22A-22D are graphs showing tumor changes over time. Tumor growth over time (FIG. 22A) for saline, DIV3W-siNT, and DIV3W-siCSNK2A1 treated animals of the multidose schedule. Tumor volume change (FIG. 22B) from treatment injection 1 through final excised volume measurement. Tumor mass (FIG. 22C) and tumor volumes (FIG. 22D) recorded after euthanasia and tumor harvesting. Data were acquired to N=5 and analyzed via one-way ANOVA analysis where * p≤0.05.

FIGS. 23A-23C CSNK2A1 mRNA and CK2α protein expression following a multi-dosing regimen of DIV3W-siCSNK2A1 in SQ ovarian tumors. (FIG. 23A) CSNK2A1 gene expression quantified via qPCR relative to saline-treated controls. (FIG. 23B) Western blot analysis of CK2α knockdown caused by CSNK2A1 gene silencing. (FIG. 23C) Immunocytochemistry of CK2α protein expression (GFP, green) in 0.5-μm tumor tissue sections with nuclei stained with Hoechst 33342 (blue). Data are mean±SEM of N=5 analyzed via one-way ANOVA, where ** p<0.01. Scale bar: 50 μm.

FIG. 24 is a graph showing Vimentin and Akt mRNA expression fold change after multi-dosing treatment with DIV3W-siCSNK2A1 relative to the saline control. Data are mean±SEM of N=4 and analyzed via one-way ANOVA. ns, not significant.

FIG. 25 is a graph showing cell viability 48-hours after treatment with DIV3W-siNT peptide complexes using MCF10a healthy, epithelial breast cells. Data are mean±SEM of three independent experiments performed in triplicate.

FIGS. 26A-26C. (FIG. 26A) Fluorescence microscopy of siRNA uptake in CAOV3 cells untreated (UNTD), treated with siNT alone, or treated with DIV3 (X) peptide-siNT-DY547 (red) complexes for 4 hours. Cells are counterstained with Hoechst (blue). Scale bar: 100 μm. (FIGS. 26B, 26C) Flow cytometric analysis of DIV3 (X)-siNT-DY547 uptake into CAOV3 cells after 4 hours. Data are mean±SEM of three independent experiments analyzed with one-way ANOVA and posthoc Tukey multiple comparisons testing, where * p<0.05, ** p<0.01, *** p<0.001, **** p<0.0001.

FIGS. 27A-27B. qPCR analysis of CSNK2A1mRNA after treatment of CAOV3 cells with DIV3W (FIG. 27A) peptide complexed with siNT or siCSNK2A1 for 48 hours. Data are mean±SEM of three independent experiments analyzed with Student's t-test within each N:P ratio, where * p<0.05 and *** p<0.001. (FIG. 27B) Western blot analysis of CSNK2A1protein product, CK2a, after treatment of CAOV3 cells with DIV3W-siCSNK2A1 for 48 hours. β-Actin expression was monitored to ensure equal protein loading.

FIGS. 28A-28C. (FIG. 28A) DIV3W complexed with siNT or siCSNK2A1 was delivered to OVCAR3 and CAOV3 cells, and the viability of the cells was quantified after 48-hour complex treatment. (FIG. 28B) CAOV3 cell percent wound heal after treatment with 80:1 DIV3W-siNT or DIV3W-siCSNK2A1 complexes over 72 hours. Scale bar: 1000 μm. (FIG. 28C) Cell migration over the course of 72 hours was quantified with ImageJ and represented as percent wound heal relative to the initial scratch distance. Data are mean±SEM of three independent experiments and were analyzed using a two-way ANOVA with Tukey's multiple comparisons posthoc test, where ** p≤0.01.

FIGS. 29A-29D depicts an approach for the design and testing of exemplary fusogenic peptide compositions of the invention. FIG. 29A exemplary peptide sequences comprising varying hydrophilic and hydrophobic residues; FIG. 29B formation of a nanoparticle complex comprising a fusogenic peptide and siRNA as an exemplary cargo; FIG. 29C schematic of inhibition of protein expression in a cell with an exemplary nanoparticle complex of the invention; FIG. 29D exemplary testing of intratumoral/intraperitoneal injections of nanoparticle complexes to reduce tumor burden.

FIGS. 30A-30D VA-LD and GF-LD peptides bind siRNA at N:P ratios of 10:1 or higher and protect siRNA in physiologically relevant environments. FIGS. 30A, 30C. Gels showing peptide-siRNA nanocomplexes at increasing N:P ratios for peptides for VA-LD (FIG. 30A) and GF-LD (FIG. 30C) incubated in either Rnase A enzyme or in 50% fetal bovine serum (FBS) visualizing siRNA bands. FIGS. 30B,30D. Gels showing increasing N:P ratios of peptide:siRNA for VA-LD (FIG. 30B) and GF-LD (FIG. 30D) from 20:1 to 80:1 N:P; white bands are indicative of intact siRNA.

FIG. 31. Graph shows ES2 ovarian cancer cell viability at VA-LD and GF-LD N:P ratio from 0 to 80:1 with non-targeting siRNA. Data representative of mean±SEM. (N=3).

FIGS. 32A-32C. Delivery of siRNA VA-LD and GD-LD peptides to ES2 ovarian cancer cells. FIG. 32A. Fluorescence microscopy image of VA-LD and GF-LD, and cy5 labeled non targeting siRNA were electrostatically complexed at increasing N:P ratios and delivered to human ovarian ES2 cells for 4 hours. Cell nuclei (Hoechst 33342), siRNA (cy5). Scale bar=100 μm. FIG. 32B Flow cytometry quantification for (FIG. 32B) mean fluorescence intensity (MFI) and (32C) cells positive for siRNA. of cells from FIG. 32A.

### DETAILED DESCRIPTION OF THE INVENTION

The present invention will now be described in more detail with reference to the accompanying drawings, in which preferred embodiments of the invention are shown. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention. All publications, patent applications, patents, patent publications and other references cited herein are incorporated by reference in their entireties for the teachings relevant to the sentence and/or paragraph in which the reference is presented.

Nucleotide sequences are presented herein by single strand only, in the 5′ to 3′ direction, from left to right, unless specifically indicated otherwise. Nucleotides and amino acids are represented herein in the manner recommended by the IUPAC-IUB Biochemical Nomenclature Commission, or (for amino acids) by either the one-letter code, or the three letter code, both in accordance with 37 C.F.R. § 1.822 and established usage.

Except as otherwise indicated, standard methods known to those skilled in the art may be used for cloning genes, amplifying and detecting nucleic acids, and the like. Such techniques are known to those skilled in the art. See, e.g., Green et al., Molecular Cloning: A Laboratory Manual 4th Ed. (Cold Spring Harbor, NY, 2012); Ausubel et al. Current Protocols in Molecular Biology (Green Publishing Associates, Inc. and John Wiley & Sons, Inc., New York).

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination. To illustrate further, if, for example, the specification indicates that a particular amino acid can be selected from A, G, I, L and/or V, this language also indicates that the amino acid can be selected from any subset of these amino acid(s) for example A, G, I or L; A, G, I or V; A or G; only L; etc. as if each such subcombination is expressly set forth herein. Moreover, such language also indicates that one or more of the specified amino acids can be disclaimed. For example, in particular embodiments the amino acid is not A, G or I; is not A; is not G or V; etc. as if each such possible disclaimer is expressly set forth herein.

### Definitions

The following terms are used in the description herein and the appended claims.

The singular forms “a” and “an” are intended to include the plural forms as well, unless the context clearly indicates otherwise.

Furthermore, the term “about,” as used herein when referring to a measurable value such as an amount of the length of a polynucleotide or polypeptide sequence, dose, time, temperature, and the like, is meant to encompass variations of 10%, 5%, 1%, 0.5%, or even 0.1% of the specified amount.

Also as used herein, “and/or” refers to and encompasses any and all possible combinations of one or more of the associated listed items, as well as the lack of combinations when interpreted in the alternative (“or”).

As used herein, the transitional phrase “consisting essentially of” is to be interpreted as encompassing the recited materials or steps and those that do not materially affect the basic and novel characteristic(s) of the claimed invention (e.g., tissue staining). Thus, the term “consisting essentially of” as used herein should not be interpreted as equivalent to “comprising.”

The term “consists essentially of” (and grammatical variants), as applied to a polypeptide sequence of this invention, means a polypeptide that consists of both the recited sequence (e.g., SEQ ID NO) and a total of ten or less (e.g., 1, 2, 3, 4, 5, 6, 7, 8, 9, or 10) additional amino acids on the N-terminal and/or C-terminal ends of the recited sequence such that the function of the polypeptide is not materially altered. The total of ten or less additional amino acids includes the total number of additional amino acids on both ends added together. The term “materially altered,” as applied to polypeptides of the invention, refers to an increase or decrease in cargo delivery activity of at least about 50% or more as compared to the activity of a polypeptide consisting of the recited sequence.

By the term “express” or “expression” of a polynucleotide coding sequence, it is meant that the sequence is transcribed, and optionally, translated. Typically, according to the present invention, expression of a coding sequence of the invention will result in production of, e.g., a siRNA or polypeptide of the invention. The entire expressed polypeptide or fragment can also function in intact cells without purification.

As used herein, the term “over-expression” or “over-expressing” refers to increased levels of a polypeptide being produced and/or increased time of expression (e.g., constitutively expressed) compared to a wild-type cell.

The term “modulate,” “modulates,” or “modulation” refers to enhancement (e.g., an increase) or inhibition (e.g., a decrease) in the specified level or activity.

The term “enhance” or “increase” refers to an increase in the specified parameter of at least about 1.25-fold, 1.5-fold, 2-fold, 3-fold, 4-fold, 5-fold, 6-fold, 8-fold, 10-fold, twelve-fold, or even fifteen-fold.

The term “inhibit” or “reduce” or grammatical variations thereof as used herein refers to a decrease or diminishment in the specified level or activity of at least about 15%, 25%, 35%, 40%, 50%, 60%, 75%, 80%, 90%, 95% or more. In particular embodiments, the inhibition or reduction results in little or essentially no detectible activity (at most, an insignificant amount, e.g., less than about 10% or even 5%).

The term “contact” or grammatical variations thereof as used with respect to a complex and a cell, refers to bringing the complex and the cell in sufficiently close proximity to each other for one to exert a biological effect on the other. In some embodiments, the term contact means binding of the complex to the cell.

By the terms “treat,” “treating,” or “treatment of” (and grammatical variations thereof) it is meant that the severity of the subject's condition is reduced, at least partially improved or stabilized and/or that some alleviation, mitigation, decrease or stabilization in at least one clinical symptom is achieved and/or there is a delay in the progression of the disease or disorder.

The terms “prevent,” “preventing,” and “prevention” (and grammatical variations thereof) refer to prevention and/or delay of the onset of a disease, disorder and/or a clinical symptom(s) in a subject and/or a reduction in the severity of the onset of the disease, disorder and/or clinical symptom(s) relative to what would occur in the absence of the methods of the invention. The prevention can be complete, e.g., the total absence of the disease, disorder and/or clinical symptom(s). The prevention can also be partial, such that the occurrence of the disease, disorder and/or clinical symptom(s) in the subject and/or the severity of onset is less than what would occur in the absence of the present invention.

A “therapeutically effective” amount as used herein is an amount that provides some improvement or benefit to the subject. Alternatively stated, a “therapeutically effective” amount is an amount that will provide some alleviation, mitigation, or decrease in at least one clinical symptom in the subject. Those skilled in the art will appreciate that the therapeutic effects need not be complete or curative, as long as some benefit is provided to the subject.

A “prevention effective” amount as used herein is an amount that is sufficient to prevent and/or delay the onset of a disease, disorder and/or clinical symptoms in a subject and/or to reduce and/or delay the severity of the onset of a disease, disorder and/or clinical symptoms in a subject relative to what would occur in the absence of the methods of the invention. Those skilled in the art will appreciate that the level of prevention need not be complete, as long as some benefit is provided to the subject.

The term “fragment,” as applied to a polypeptide, will be understood to mean an amino acid sequence of reduced length relative to a reference polypeptide or amino acid sequence and comprising, consisting essentially of, and/or consisting of an amino acid sequence of contiguous amino acids identical or almost identical (e.g., 90%, 92%, 95%, 98%, 99% identical) to the reference polypeptide or amino acid sequence. Such a polypeptide fragment according to the invention may be, where appropriate, included in a larger polypeptide of which it is a constituent. In some embodiments, such fragments can comprise, consist essentially of, and/or consist of peptides having a length of at least about 4, 6, 8, 10, 12, 15, 20, 25, 30, 35, 40, 45, 50, 75, 100, 150, 200, or more consecutive amino acids of a polypeptide or amino acid sequence according to the invention. In other embodiments, such fragments can comprise, consist essentially of, and/or consist of peptides having a length of less than about 15, 14, 13, 12, 11, 10, 9, 8, 7, 6, 5, 4, or less consecutive amino acids of a peptide or amino acid sequence according to the invention.

As used herein, the terms “protein” and “polypeptide” are used interchangeably and encompass both peptides and proteins, unless indicated otherwise.

The “N-terminus” of a polypeptide is any portion of the polypeptide that starts from the N-terminal amino acid residue and continues to a maximum of the midpoint of the polypeptide.

The “C-terminus” of a polypeptide is any portion of the polypeptide that starts from the C-terminal amino acid residue and continues to a maximum of the midpoint of the polypeptide.

A “fusion protein” is a polypeptide produced when two heterologous nucleotide sequences or fragments thereof coding for two (or more) different polypeptides not found fused together in nature are fused together in the correct translational reading frame. Illustrative fusion polypeptides include fusions of a peptide of the invention (or a fragment thereof) to all or a portion of glutathione-S-transferase, maltose-binding protein, or a reporter protein (e.g., Green Fluorescent Protein, β-glucuronidase, β-galactosidase, luciferase, etc.), hemagglutinin, c-myc, FLAG epitope, an Fc region, etc.

A “fusogenic peptide” is a peptide designed with a core amphiphilic sequence that enables fusion of the fusogenic peptide with the endosomal membrane. Additional peptides can be attached to the fusogenic peptide confering additional properties to the fusogenic peptide (e.g., a positively-charged amino acid sequence for carrying a cargo for endosomal delivery or a targeting peptide sequence for targeting specific cell types).

“Amphipathic properties” describes a peptide having both hydrophilic and hydrophobic residues. Peptides with amphipathic properties described herein are able to intereact with the cell membrane via the hydrophobic amino acid residue affinity for the lipic bilayer membrane, undergo a conformational change in the endosome, and insert itself into the endosomal membrane, causing destabilization and breakdown of the endosome.

A “N/P ratio” is a measure of the number of amine groups present in the peptide times the number of peptides (e.g., concentration) divided by the number of phosphates in a nucleic acid (e.g., siRNA) times the number of nucleic acids present, creating a charge and/or molar content ratio between the two.

A “target peptide” or “targeting peptide” is a peptide designed to preferentially bind to a specific molecule (e.g., protein, lipid, glycosylation site) associated with a specific tissue or cell type (e.g., ovarian tissue, breast tissue, cervical tissue, cancer cells).

As used herein, a “functional” polypeptide or “functional fragment” is one that substantially retains at least one biological activity normally associated with that polypeptide (e.g., cargo delivery). In particular embodiments, the “functional” polypeptide or “functional fragment” substantially retains all of the activities possessed by the unmodified polypeptide. By “substantially retains” biological activity, it is meant that the polypeptide retains at least about 50%, 60%, 75%, 85%, 90%, 95%, 97%, 98%, 99%, or more, of the biological activity of the native polypeptide (and can even have a higher level of activity than the native polypeptide). A “non-functional” polypeptide is one that exhibits little or essentially no detectable biological activity normally associated with the polypeptide (e.g., at most, only an insignificant amount, e.g., less than about 10% or even 5%). Biological activities such as membrane binding and cargo delivery activity can be measured using assays that are well known in the art and as described herein.

As used herein, “nucleic acid,” “nucleotide sequence,” and “polynucleotide” are used interchangeably and encompass both RNA and DNA, including cDNA, genomic DNA, mRNA, synthetic (e.g., chemically synthesized) DNA or RNA and chimeras of RNA and DNA. The term nucleic acid refers to a chain of nucleotides without regard to length of the chain. The nucleic acid can be double-stranded or single-stranded. Where single-stranded, the nucleic acid can be a sense strand or an antisense strand. The nucleic acid can be synthesized using oligonucleotide analogs or derivatives (e.g., inosine or phosphorothioate nucleotides). Such oligonucleotides can be used, for example, to prepare nucleic acids that have altered base-pairing abilities or increased resistance to nucleases. The present invention further provides a nucleic acid that is the complement (which can be either a full complement or a partial complement) of a nucleic acid or nucleotide sequence of this invention. When dsRNA is produced synthetically, less common bases, such as inosine, 5-methylcytosine, 6-methyladenine, hypoxanthine and others can also be used for antisense, dsRNA, and ribozyme pairing. For example, polynucleotides that contain C-5 propyne analogues of uridine and cytidine have been shown to bind RNA with high affinity and to be potent antisense inhibitors of gene expression. Other modifications, such as modification to the phosphodiester backbone, or the 2′-hydroxy in the ribose sugar group of the RNA can also be made.

An “isolated polynucleotide” is a nucleotide sequence (e.g., DNA or RNA) that is not immediately contiguous with nucleotide sequences with which it is immediately contiguous (one on the 5′ end and one on the 3′ end) in the naturally occurring genome of the organism from which it is derived. Thus, in one embodiment, an isolated nucleic acid includes some or all of the 5′ non-coding (e.g., promoter) sequences that are immediately contiguous to a coding sequence. The term therefore includes, for example, a recombinant DNA that is incorporated into a vector, into an autonomously replicating plasmid or virus, or into the genomic DNA of a prokaryote or eukaryote, or which exists as a separate molecule (e.g., a cDNA or a genomic DNA fragment produced by PCR or restriction endonuclease treatment), independent of other sequences. It also includes a recombinant DNA that is part of a hybrid nucleic acid encoding an additional polypeptide or peptide sequence. An isolated polynucleotide that includes a gene is not a fragment of a chromosome that includes such gene, but rather includes the coding region and regulatory regions associated with the gene, but no additional genes naturally found on the chromosome.

The term “isolated” can refer to a nucleic acid, nucleotide sequence or polypeptide that is substantially free of cellular material, viral material, and/or culture medium (when produced by recombinant DNA techniques), or chemical precursors or other chemicals (when chemically synthesized). Moreover, an “isolated fragment” is a fragment of a nucleic acid, nucleotide sequence or polypeptide that is not naturally occurring as a fragment and would not be found in the natural state. “Isolated” does not mean that the preparation is technically pure (homogeneous), but it is sufficiently pure to provide the polypeptide or nucleic acid in a form in which it can be used for the intended purpose.

An “isolated cell” refers to a cell that is separated from other components with which it is normally associated in its natural state. For example, an isolated cell can be a cell in culture medium and/or a cell in a pharmaceutically acceptable carrier of this invention. Thus, an isolated cell can be delivered to and/or introduced into a subject. In some embodiments, an isolated cell can be a cell that is removed from a subject and manipulated as described herein ex vivo and then returned to the subject.

A “vector” is any nucleic acid molecule for the cloning of and/or transfer of a nucleic acid into a cell. A vector may be a replicon to which another nucleotide sequence may be attached to allow for replication of the attached nucleotide sequence. A “replicon” can be any genetic element (e.g., plasmid, phage, cosmid, chromosome, viral genome) that functions as an autonomous unit of nucleic acid replication in vivo, i.e., capable of replication under its own control. The term “vector” includes both viral and nonviral (e.g., plasmid) nucleic acid molecules for introducing a nucleic acid into a cell in vitro, ex vivo, and/or in vivo. A large number of vectors known in the art may be used to manipulate nucleic acids, incorporate response elements and promoters into genes, etc. For example, the insertion of the nucleic acid fragments corresponding to response elements and promoters into a suitable vector can be accomplished by ligating the appropriate nucleic acid fragments into a chosen vector that has complementary cohesive termini. Alternatively, the ends of the nucleic acid molecules may be enzymatically modified or any site may be produced by ligating nucleotide sequences (linkers) to the nucleic acid termini. Such vectors may be engineered to contain sequences encoding selectable markers that provide for the selection of cells that contain the vector and/or have incorporated the nucleic acid of the vector into the cellular genome. Such markers allow identification and/or selection of host cells that incorporate and express the proteins encoded by the marker. A “recombinant” vector refers to a viral or non-viral vector that comprises one or more heterologous nucleotide sequences (i.e., transgenes), e.g., two, three, four, five or more heterologous nucleotide sequences.

Viral vectors have been used in a wide variety of gene delivery applications in cells, as well as living animal subjects. Viral vectors that can be used include, but are not limited to, retrovirus, lentivirus, adeno-associated virus, poxvirus, alphavirus, baculovirus, vaccinia virus, herpes virus, Epstein-Barr virus, and adenovirus vectors. Non-viral vectors include plasmids, liposomes, electrically charged lipids (cytofectins), nucleic acid-protein complexes, and biopolymers. In addition to a nucleic acid of interest, a vector may also comprise one or more regulatory regions, and/or selectable markers useful in selecting, measuring, and monitoring nucleic acid transfer results (delivery to specific tissues, duration of expression, etc.).

The term “transfection” or “transduction” means the uptake of exogenous or heterologous nucleic acid (RNA and/or DNA) by a cell. A cell has been “transfected” or “transduced” with an exogenous or heterologous nucleic acid when such nucleic acid has been introduced or delivered inside the cell. A cell has been “transformed” by exogenous or heterologous nucleic acid when the transfected or transduced nucleic acid imparts a phenotypic change in the cell and/or a change in an activity or function of the cell. The transforming nucleic acid can be integrated (covalently linked) into chromosomal DNA making up the genome of the cell or it can be present as a stable plasmid.

As used herein, the term “gene” refers to a nucleic acid molecule capable of being used to produce mRNA, antisense RNA, miRNA, and the like. Genes may or may not be capable of being used to produce a functional protein. Genes can include both coding and non-coding regions (e.g., introns, regulatory elements, promoters, enhancers, termination sequences and 5′ and 3′ untranslated regions). A gene may be “isolated” by which is meant a nucleic acid that is substantially or essentially free from components normally found in association with the nucleic acid in its natural state. Such components include other cellular material, culture medium from recombinant production, and/or various chemicals used in chemically synthesizing the nucleic acid.

As used herein, “complementary” polynucleotides are those that are capable of base pairing according to the standard Watson-Crick complementarity rules. Specifically, purines will base pair with pyrimidines to form a combination of guanine paired with cytosine (G: C) and adenine paired with either thymine (A: T) in the case of DNA, or adenine paired with uracil (A: U) in the case of RNA. For example, the sequence “A-G-T” binds to the complementary sequence “T-C-A.” It is understood that two polynucleotides may hybridize to each other even if they are not completely complementary to each other, provided that each has at least one region that is substantially complementary to the other.

The terms “complementary” or “complementarity,” as used herein, refer to the natural binding of polynucleotides under permissive salt and temperature conditions by base-pairing. Complementarity between two single-stranded molecules may be “partial,” in which only some of the nucleotides bind, or it may be complete when total complementarity exists between the single stranded molecules. The degree of complementarity between nucleic acid strands has significant effects on the efficiency and strength of hybridization between nucleic acid strands.

As used herein, the terms “substantially complementary” or “partially complementary” mean that two nucleic acid sequences are complementary at least about 50%, 60%, 70%, 80% or 90% of their nucleotides. In some embodiments, the two nucleic acid sequences can be complementary at least at 85%, 90%, 95%, 96%, 97%, 98%, 99% or more of their nucleotides. The terms “substantially complementary” and “partially complementary” can also mean that two nucleic acid sequences can hybridize under high stringency conditions and such conditions are well known in the art.

As used herein, “heterologous” refers to a nucleic acid sequence that either originates from another species or is from the same species or organism but is modified from either its original form or the form primarily expressed in the cell. Thus, a nucleotide sequence derived from an organism or species different from that of the cell into which the nucleotide sequence is introduced, is heterologous with respect to that cell and the cell's descendants. In addition, a heterologous nucleotide sequence includes a nucleotide sequence derived from and inserted into the same natural, original cell type, but which is present in a non-natural state, e.g., a different copy number, and/or under the control of different regulatory sequences than that found in nature.

“Introducing” in the context of a cell or organism means presenting the nucleic acid molecule to the organism and/or cell in such a manner that the nucleic acid molecule gains access to the interior of a cell. Where more than one nucleic acid molecule is to be introduced these nucleic acid molecules can be assembled as part of a single polynucleotide or nucleic acid construct, or as separate polynucleotide or nucleic acid constructs, and can be located on the same or different nucleic acid constructs. Accordingly, these polynucleotides can be introduced into cells in a single transformation event or in separate transformation events. Thus, the term “transformation” as used herein refers to the introduction of a heterologous nucleic acid into a cell. Transformation of a cell may be stable or transient.

“Transient transformation” in the context of a polynucleotide means that a polynucleotide is introduced into the cell and does not integrate into the genome of the cell.

By “stably introducing” or “stably introduced” in the context of a polynucleotide introduced into a cell, it is intended that the introduced polynucleotide is stably incorporated into the genome of the cell, and thus the cell is stably transformed with the polynucleotide.

“Stable transformation” or “stably transformed” as used herein means that a nucleic acid molecule is introduced into a cell and integrates into the genome of the cell. As such, the integrated nucleic acid molecule is capable of being inherited by the progeny thereof, more particularly, by the progeny of multiple successive generations. “Genome” as used herein includes the nuclear and mitochondrial genome, and therefore includes integration of the nucleic acid into, for example, the mitochondrial genome. Stable transformation as used herein can also refer to a transgene that is maintained extrachromasomally, for example, as a minichromosome.

Transient transformation may be detected by, for example, an enzyme-linked immunosorbent assay (ELISA) or Western blot, which can detect the presence of a peptide or polypeptide encoded by one or more transgene introduced into an organism or, in the case of, for example, siRNA, detect the reduction in expression of a protein by the host cell or organism. Stable transformation of a cell can be detected by, for example, a Southern blot hybridization assay of genomic DNA of the cell with nucleic acid sequences which specifically hybridize with a nucleotide sequence of a transgene introduced into an organism. Stable transformation of a cell can be detected by, for example, a Northern blot hybridization assay of RNA of the cell with nucleic acid sequences which specifically hybridize with a nucleotide sequence of a transgene introduced into an organism. Stable transformation of a cell can also be detected by, e.g., a polymerase chain reaction (PCR) or other amplification reactions as are well known in the art, employing specific primer sequences that hybridize with target sequence(s) of a transgene, resulting in amplification of the transgene sequence, which can be detected according to standard methods Transformation can also be detected by direct sequencing and/or hybridization protocols well known in the art.

As used herein “sequence identity” refers to the extent to which two optimally aligned polynucleotide or polypeptide sequences are invariant throughout a window of alignment of components, e.g., nucleotides or amino acids. “Identity” can be readily calculated by known methods including, but not limited to, those described in: Computational Molecular Biology (Lesk, A. M., ed.) Oxford University Press, New York (1988); Biocomputing: Informatics and Genome Projects (Smith, D. W., ed.) Academic Press, New York (1993); Computer Analysis of Sequence Data, Part I (Griffin, A. M., and Griffin, H. G., eds.) Humana Press, New Jersey (1994); Sequence Analysis in Molecular Biology (von Heinje, G., ed.) Academic Press (1987); and Sequence Analysis Primer (Gribskov, M. and Devereux, J., eds.) Stockton Press, New York (1991).

As used herein, the term “substantially identical” or “corresponding to” means that two nucleic acid sequences or two polypeptide sequences have at least 60%, 70%, 80% or 90% sequence identity. In some embodiments, the two nucleic acid sequences can have at least 85%, 90%, 95%, 96%, 97%, 98%, 99% or 100% of sequence identity.

An “identity fraction” for aligned segments of a test sequence and a reference sequence is the number of identical components which are shared by the two aligned sequences divided by the total number of components in reference sequence segment, i.e., the entire reference sequence or a smaller defined part of the reference sequence.

As used herein, the term “percent sequence identity” or “percent identity” refers to the percentage of identical nucleotides in a linear polynucleotide sequence of a reference (“query”) polynucleotide molecule (or its complementary strand) as compared to a test (“subject”) polynucleotide molecule (or its complementary strand) when the two sequences are optimally aligned (with appropriate nucleotide insertions, deletions, or gaps totaling less than 20 percent of the reference sequence over the window of comparison). In some embodiments, “percent identity” can refer to the percentage of identical amino acids in an amino acid sequence.

Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and optionally by computerized implementations of these algorithms such as GAP, BESTFIT, FASTA, and TFASTA available as part of the GCG® Wisconsin Package® (Accelrys Inc., Burlington, Mass.). Percent sequence identity is represented as the identity fraction multiplied by 100. The comparison of one or more polynucleotide sequences may be to a full-length polynucleotide sequence or a portion thereof, or to a longer polynucleotide sequence. For purposes of this invention “percent identity” may also be determined using BLASTX version 2.0 for translated nucleotide sequences and BLASTN version 2.0 for polynucleotide sequences.

The percent of sequence identity can be determined using the “Best Fit” or “Gap” program of the Sequence Analysis Software Package™ (Version 10; Genetics Computer Group, Inc., Madison, Wis.). “Gap” utilizes the algorithm of Needleman and Wunsch (Needleman and Wunsch, J Mol. Biol. 48:443-453, 1970) to find the alignment of two sequences that maximizes the number of matches and minimizes the number of gaps. “BestFit” performs an optimal alignment of the best segment of similarity between two sequences and inserts gaps to maximize the number of matches using the local homology algorithm of Smith and Waterman (Smith and Waterman, Adv. Appl. Math., 2:482-489, 1981, Smith et al., Nucleic Acids Res. 11:2205-2220, 1983).

Useful methods for determining sequence identity are also disclosed in Guide to Huge Computers (Martin J. Bishop, ed., Academic Press, San Diego (1994)), and Carillo, H., and Lipton, D., (Applied Math 48: 1073 (1988)). More particularly, preferred computer programs for determining sequence identity include but are not limited to the Basic Local Alignment Search Tool (BLAST) programs which are publicly available from National Center Biotechnology Information (NCBI) at the National Library of Medicine, National Institute of Health, Bethesda, Md. 20894; see BLAST Manual, Altschul et al., NCBI, NLM, NIH; (Altschul et al., J. Mol. Biol. 215:403-410 (1990)); version 2.0 or higher of BLAST programs allows the introduction of gaps (deletions and insertions) into alignments; for peptide sequence BLASTX can be used to determine sequence identity; and, for polynucleotide sequence BLASTN can be used to determine sequence identity.

As used herein, “RNAi” or “RNA interference” refers to the process of sequence-specific post-transcriptional gene silencing, mediated by double-stranded RNA (dsRNA). As used herein, “dsRNA” refers to RNA that is partially or completely double stranded. Double stranded RNA is also referred to as small interfering RNA (siRNA), small interfering nucleic acid (siNA), microRNA (miRNA), and the like. In the RNAi process, dsRNA comprising a first (antisense) strand that is complementary to a portion of a target gene and a second (sense) strand that is fully or partially complementary to the first antisense strand is introduced into an organism. After introduction into the organism, the target gene-specific dsRNA is processed into relatively small fragments (siRNAs) and can subsequently become distributed throughout the organism, leading to a phenotype that, over the period of a generation, may come to closely resemble the phenotype arising from a complete or partial deletion of the target gene.

MicroRNAs (miRNAs) are non-protein coding RNAs, generally of between about 18 to about 25 nucleotides in length. These miRNAs direct cleavage in trans of target transcripts, negatively regulating the expression of genes involved in various regulation and development pathways (Bartel, Cell, 116:281-297 (2004); Zhang et al. Dev. Biol. 289:3-16 (2006)). As such, miRNAs have been shown to be involved in different aspects of growth and development as well as in signal transduction and protein degradation. Since the first miRNAs were discovered in plants (Reinhart et al. Genes Dev. 16:1616-1626 (2002), Park et al. Curr. Biol. 12:1484-1495 (2002)) many hundreds have been identified. Many microRNA genes (MIR genes) have been identified and made publicly available in a database (miRBase; microrna.sanger.ac.uk/sequences). miRNAs are also described in U.S. Patent Publications 2005/0120415 and 2005/144669A1, the entire contents of which are incorporated by reference herein.

Genes encoding miRNAs yield primary miRNAs (termed a “pri-miRNA”) of 70 to 300 bp in length that can form imperfect stem-loop structures. A single pri-miRNA may contain from one to several miRNA precursors. In animals, pri-miRNAs are processed in the nucleus into shorter hairpin RNAs of about 65 nt (pre-miRNAs) by the RNaseIII enzyme Drosha and its cofactor DGCR8/Pasha. The pre-miRNA is then exported to the cytoplasm, where it is further processed by another RNaseIII enzyme, Dicer, releasing a miRNA/miRNA* duplex of about 22 nt in size. Many reviews on microRNA biogenesis and function are available, for example, see, Bartel Cell 116:281-297 (2004), Murchison et al. Curr. Opin. Cell Biol. 16:223-229 (2004), Dugas et al. Curr. Opin. Plant Biol. 7:512-520 (2004) and Kim Nature Rev. Mol. Cell Biol. 6:376-385 (2005).

Chemical and/or biological modifications may be conducted to optimize pharmacodynamics or water solubility of the peptide or to lower its side effects. For example, PEGylation, PASylation and/or HESylation may be applied to slow down renal clearance and thereby increase plasma half-life time of the peptide. Modification of the N-terminal and/or C-terminal residue may inhibit peptide degradation. Additionally, or alternatively, a modification may add a different functionality to the peptide, e.g., a toxin to more efficiently combat cancer cells, or a detection molecule for diagnostic purposes.

Glycosylation refers to a process that attaches carbohydrates to proteins. In biological systems, this process is performed enzymatically within the cell as a form of co-translational and/or post-translational modification. A protein, here the fusogenic peptide, can also be chemically glycosylated. Typical glycosylation patterns include, but are not limited to (i) N-linked to a nitrogen of asparagine or arginine side-chains; (ii)O-linked to the hydroxy oxygen of serine, threonine, tyrosine, hydroxylysine, or hydroxyproline side-chains; (iii) involves the attachment of xylose, fucose, mannose, and N-acetylglucosamine to a phospho-serine; or (iv) in form of C-mannosylation wherein a mannose sugar is added to a tryptophan residue found in a specific recognition sequence. Glycosylation patterns can, e.g., be controlled by choosing appropriate cell lines, culturing media, protein engineering manufacturing modes and process strategies (HOSSLER, P. Optimal and consistent protein glycosylation in mammalian cell culture. Glycobiology 2009, vol. 19, no. 9, p. 936-949.).

Protein engineering to control or alter the glycosylation pattern may involve the deletion and/or the addition of one or more glycosylation sites. The creation of glycosylation sites can conveniently be accomplished by introducing the corresponding enzymatic recognition sequence into the amino acid sequence of the fusogenic peptide or by adding or substituting one or more of the above enumerated amino acid residues.

### Fusogenic Polypeptides

A set of rationally designed fusogenic peptides have been developed for complexation with cargo to be delivered into cells. These novel fusogenic peptides were designed to address two significant barriers to siRNA delivery: insufficient cellular uptake and endosomal entrapment. Each peptide comprises a first peptide sequence and a second peptide sequence and was designed to maintain amphipathic properties and include a sequence of positive amino acid residues (e.g., D-arginine residues) responsible for cargo complexation. Accordingly, one aspect of the invention relates to fusogenic peptides for the delivery of cargo to a cell. In one embodiment, the fusogenic peptides have a first peptide sequence with amphipathic properties and a second peptide sequence comprising postively-charged amino acid residues. The fusogenic peptide may further comprise one or more linkers.

In some embodiments, the first peptide is linked to the second peptide by a linker. In some embodiments, the first peptide is further linked to a targeting peptide. In some embodiments, the first peptide is linked to the targeting peptide by a linker. The linker can be a flexible or cleavable linker. Flexible and cleavable linkers and design considerations are known in the art. See, e.g., Chen et al., Adv Drug Deliv Rev. 2013 Oct. 15; 65 (10): 1357-1369; doi: 10.1016/j.addr.2012.09.039. In some embodiments, flexible linkers can be used that can each independently be a series of glycine residues. The flexible linkers can each independently be from 1 to 10 residues, e.g., 1, 2, 3, 4, 5, 6, 7, 8, 9, or 10 residues, from 3 to 7 residues, 3 residues, 4 residues, or 5 residues in length. In some embodiments, cleavable linkers are used. Cleavable linkers can comprise one or more repeats of the amino acid residues VA or GFLG (SEQ ID NO:7) and can be cleaved by cathespin B. In one embodiment, the cleavable linker links a targeting peptide to the first peptide sequence in the fusogenic peptide so that the targeting peptide is removed after the fusogenic peptide enters a cell. In one embodiment, the linker between the targeting peptide and the first peptide is a cleavable linker and the linker between the first peptide and the seond peptide is a flexible linker.

In some embodiments, the first peptide is located near or at the N-terminal end of the fusogenic peptide and the second peptide is located near or at the C-terminal end of the fusogenic peptide. In other embodiments, the targeting peptide is located near or at the N-terminal end of the fusogenic peptide, the second peptide is located near or at the C-terminal end of the fusogenic peptide, and the first peptide is located between the targeting peptide and the second peptide. In an embodiment, the fusogenic peptide comprises in order, from the N-terminal end to the C-terminal end, the first peptide sequence with amphipathic properties, a linker, and a second peptide sequence comprising two or more positively-charged amino acid residues. In an embodiment, the fusogenic peptide comprises in order, from the N-terminal end to the C-terminal end, a targeting peptide, a linker, the first peptide sequence with amphipathic properties, optionally a linker, and a second peptide sequence comprising two or more positively-charged amino acid residues.

The first peptide sequence can be any length suitable with ampipathic properties. In some embodiments, the first peptide sequence imparts, in whole or in part, capabilities of interacting with the cell membrane, induction of endocytosis, insertion into the endosomal membrane, or any combination thereof. In an embodiment, the first peptide sequence with ampipathic properties is from 8 to 38 residues in length, e.g., about 8, 9, 10, 11, 12, 13, 14, 15, 16, 17, 18, 19, 20, 21, 22, 23, 24, 25, 26, 27, 28, 29, 30, 31, 32, 33, 34, 35, 36, 37 or 38 residues in length or any range therein.

In embodiments, the first peptide sequence with ampipathic properties comprises one or more sequences of DIVX wherein X is any amino acid. In some embodiments, the amphipathic sequence of the fusogenic polypeptide includes from 2 to 6 or from 3 to 4 repeats of a DIV (X) amino acid sequence, wherein each X is independently selected from alanine, histidine, and tryptophan. In some embodiments, the fusogenic polypeptide includes more than 6 repeats of a DIV (X) sequence, wherein each X is independently selected from alanine, histidine, and tryptophan. Each DIV (X) sequence repeat within a peptide may be the same or different. In one embodiment, the fusogenic peptide is from 8-38, from 18 to 38, from 20 to 35, or from 25 to 33 residues in length. In one embodiment, the fusogenic peptides are DIV (X) peptides (SEQ ID NO: 6) wherein X is selected from alanine, histidine, and tryptophan and the arginine reidues are D-arginine. The arginine residues present as D-amino acids are underlined.

DIV(X)
(SEQ ID NO: 6)
WEADI VADIV XDIVA DIVAG GGRRR RRRRR R

In another embodiment, the fusogenic peptides are DIVA3 (SEQ ID NO:1), DIV3H (SEQ ID NO:2), or DIV3W (SEQ ID NO:3).

DIVA3
(SEQ ID NO: 1)
WEADI VADIV ADIVA GGGRR RRRRR RR
DIV3H
(SEQ ID NO: 2)
WEADI VADIV HDIVA DIVAG GGRRR RRRRR R
DIV3W
(SEQ ID NO: 3)
WEADI VADIV WDIVA DIVAG GGRRR RRRRR R

In some embodiments, the fusogenic polypeptide comprises, consists essentially of, or consists of an amino acid sequence that is at least 70% identical, e.g., at least 75%, 80%, 85%, 90%, 95%, 96%, 97%, 98%, or 99% identical to DIVA3 (SEQ ID NO:1), DIV3H (SEQ ID NO:2), or DIV3W (SEQ ID NO:3).

The second peptide sequence may be any length that is suitable for binding a cargo, e.g., from 6-16, 7-14, 8-11, or 9 residues in length. The second peptide sequence may be 6, 7, 8, 9, 10, 11, 12, 13, 14, 15, or 16 amino acid residues in length or any range therein. The second peptide sequence may contain all or substantially all (at least 70%, 75%, 80%, 85%, 90%, or 95%) positively-charged amino acids. Positively-charged amino acids include arginine, histidine, and lysine. The amino acid residues may be naturally occurring amino acids or non-naturally occurring amino acids, including amino acid residues modified to comprise positively charged side chains. The amino acid residues may be in the D orientation. In an aspect, the second peptide sequence comprises two or more positively charged amino acid residues for binding the cargo. In an aspect, at least one of the two or more positively charged residues for binding the cargo comprises arginine. In an aspect, the arginine is D-arginine.

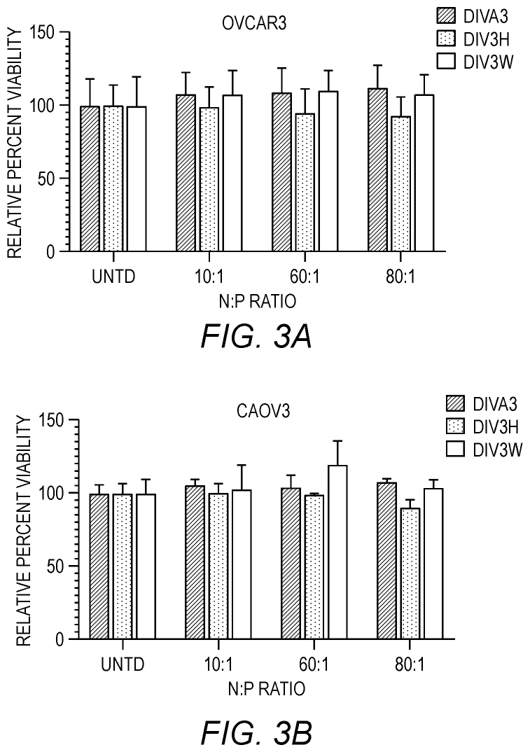

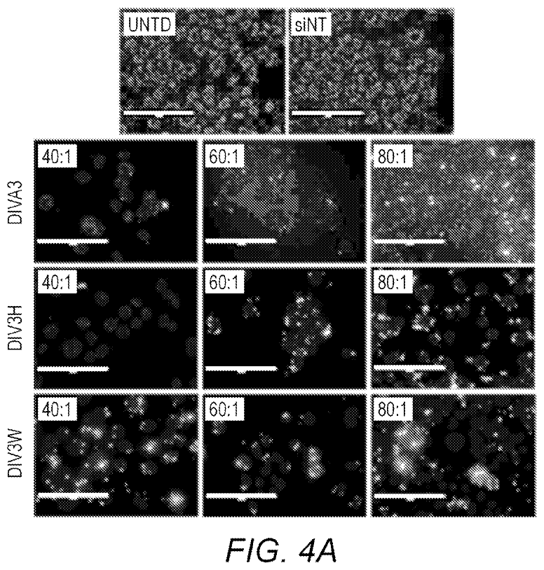

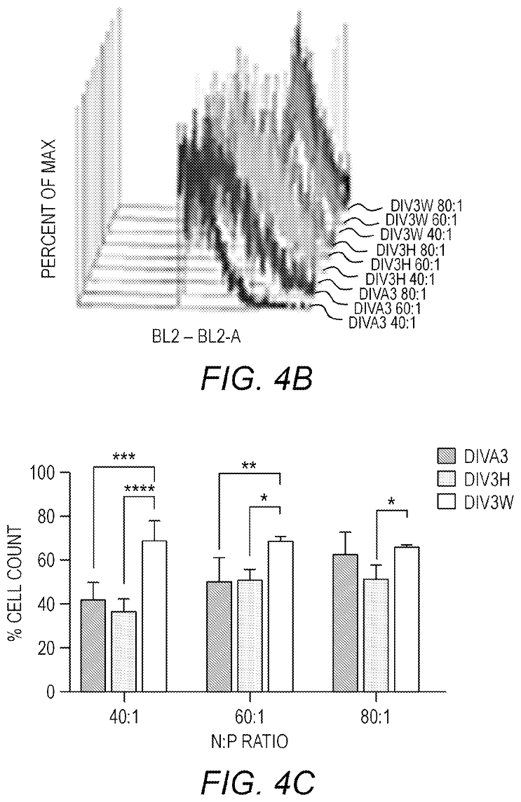

To increase cell and tissue specificity, a targeting peptide can be included within the fusogenic delivery system so that the fusogenic peptide binds to a selected target (e.g., cancer cells). The tandem peptide comprising the targeting peptide will not only enhance cellular uptake, but may also enhance the fusogenicity of the peptide to cause endosomal membrane destruction and release of bioactive cargo such as siRNAs. Accordingly, in one aspect of the invention, the fusogenic peptide further comprises a targeting peptide. The targeting peptide can be designed for a cell surface receptor or transmembrane protein target. In an aspect, the targeting peptide can be designed for two or more targets, for example, to simultaneously target different tumor cell surface receptors. Example peptide-binding receptors that have been studied in cancer treatment that may be used for designing target peptides are known in the art, including integrins, epidermal growth factor receptor, G protein-coupled receptors, gonadotropin-releasing hormone receptor, bombesin receptor family, and neuropeptide Y receptor family. See, e.g., Hoppenz et al., Front. Chem., 2020; Volume 8 doi: 10.3389/fchem.2020.00571, incorporated by reference herein in its entirety, with specific reference to Table 1. In one embodiment, the targeting sequence is a targeting peptide for HER2. See, Geng et al., Theranostics. 2016; 6 (8): 1261-1273, doi: 10.7150/thno. 14302, and FIGS. 3B-3C and FIGS. 4A-4B for exemplary HER2 targeting peptide sequences, specifically incorporated herein by reference. In one embodiment, the targeting sequence is a targeting peptide for the luteinizing hormone releasing hormone (LHRH) receptor. In some embodiments, the fusogenic peptide is longer when a targeting sequence is included. Accordingly, the fusogenic peptide together with the targeting peptide may be from 18 to 68, from 28 to 58, or from 35 to 55 residues in length. In other embodiments, the fusogenic peptide together with the targeting peptide is more than 68 residues in length. In one embodiment, the fusogenic peptide is the DIV3W polypeptide including a LHRH receptor targeting peptide (SEQ ID NO:4) linked by a flexible linker to yield a LHRD-DIV3W peptide. In an aspect, the fusogenic peptide is the DIV3W polypeptide including a LHRH receptor targeting peptide linked by a glycine linker to yield a LHRH-DIV3W polypeptide according to SEQ ID NO:5. The leucine and arginine residues present as D-amino acids are underlined. The lowercase p indicates that the N-terminal glycine is in the pyro form.

LHRH receptor targeting peptide
(SEQ ID NO: 4)
pGHWSY LLRP
LHRH-DIV3W
(SEQ ID NO: 5)
pGHWSY LLRPG GGGGW EADIV ADIVW DIVAD IVAGG GRRRR
RRRRR

In one embodiment, the fusogenic peptide is the DIV3W polypeptide including a LHRH receptor targeting peptide (SEQ ID NO:4) linked by a cleavable linker to yield LHRH-DIV3W polypeptide. In an aspect, the fusogenic peptide is the DIV3W polypeptide including a LHRH receptor targeting peptide (SEQ ID NO:4) linked by a cleavable linker comprising VA to yield an LHRH-DIV3W polypeptide according to SEQ ID NO:10. In an aspect, the fusogenic peptide is the DIV3W polypeptide including a LHRH receptor targeting peptide (SEQ ID NO:4) linked by a cleavable linker comprising GFLG (SEQ ID NO:7) to yield an LHRH-DIV3W polypeptide, GF-LD, according to SEQ ID NO:11. The leucine and arginine residues present as D-amino acids are underlined. The lowercase p indicates that the N-terminal glycine is in the pyro form.

VA-LD Peptide sequence
(SEQ ID NO: 10)
pGHWSY-LLRPGGGVAWEADIVADIVWDIVADIVAGGG-RRRRRRRRR
GF-LD peptide sequence
(SEQ ID NO: 11)
pGHWSY-LLRPGGGGFLGWEADIVADIVWDIVADIVAGGG-
RRRRRRRRR

In some embodiments, the fusogenic polypeptide comprises, consists essentially of, or consists of an amino acid sequence that is at least 70% identical, e.g., at least 75%, 80%, 85%, 90%, 95%, 96%, 97%, 98%, or 99% identical to LHRH-DIV3W (SEQ ID NO:5), VA-LD (SEQ ID NO: 10) or GF-LD (SEQ ID NO: 11).

### Complexes of the Fusogenic Peptides

The fusogenic peptides are designed to include a sequence of positively-charged amino acid residues responsible for cargo complexation. Accordingly, one aspect of the invention relates to the formation of a nanoparticle complex including the fusogenic peptide and a cargo. In some embodiments, the nanoparticle complex has a hydrodynamic diameter from about 60 to about 225 nm, from about 75 to about 175 nm, or from about 80 nm to about 150 nm. In other embodiments, the nanoparticle complex has a hydrodynamic diameter larger than about 225 nm (e.g., when a targeting peptide is attached to the fusogenic peptide and accordingly increases the hydrodynamic diameter).

A further aspect of the invention relates to a nanoparticle complex including the fusogenic peptide and a cargo. The cargo may be any molecule with negative charges that is capable of binding the second peptide of the fusogenic peptide. In an aspect, the cargo is a biologically active agent, imaging agent, or therapeutic agent. Example cargos may be a nucleic acid, a protein, a complex of a nucleic acid and a protein, a carbohydrate, a lipid, or a small molecule. The cargo may be formulated as a salt. One or two or more different cargos may be delivered in a complex with the fusogenic peptides described herein. The cargo can be selected for the treatment of a disease or disorder, for the detection of a disease or disorder, or for monitoring of a disease or disorder. In one embodiment, the cargo targets an oncogene. In one embodiment, the cargo targets the CSNK2A1 gene or the HER2 gene. In an aspect, the cargo inhibits expression of an oncoprotein encoded by an oncogene. In an example embodiment, the cargo is an mRNA for delivery to a cell to increase expression of one or more proteins. In an embodiment, the cargo is an antisense oligonucleotide or siRNA for delivery to a cell to decrease expression or achieve gene silencing of one or more target sequences. The cargo may be an imaging agent or detectable label or may be a molecule comprising an imaging agent or detectable label, that can be delivered to a cell or tissue. In an aspect, the cargo is a genetic modulating agent such as a CRISPR-Cas system comprising a CRISPR-Cas protein, or a polynucleotide encoding the CRISPR-Cas protein, and a guide sequence specific for a target sequence that can be utilized for a variety of gene editing applications. One or both of the CRISPR-Cas system components can be provided in the same or in different particles. Accordingly, the particles comprising cargo will find use in therapeutic, detection, diagnostic, and other applications. In some embodiments, the cargo is a nucleic acid (e.g., siRNA, mRNA). In some embodiments, the cargo is a chemotherapeutic agent.

### Biologically Active Agents

A biologically active agent, i.e., an agent that modulates an effect or activity in a cell, tissue, organ, or other biological media such as biological fluid, includes nucleic acids, proteins, small molecules, carbohydrates, lipids and complexes, salts thereof, and combinations thereof. The biologically active agent can be a genetic modifying agent. Representative nucleic acids include DNA, RNA, transposon DNA, antisense nucleic acids, ribozymes, plasmids, expression constructs, and RNA, such as mRNA, guide RNA, tRNA, ribosomal RNA, small nucleolar RNA, antisense oligonucleotides, or RNAi, such as siRNA, shRNA, and miRNA.

RNAi therapeutic agents comprise a polynucleotide that is complementary to a portion of the target sequence mRNA. In an example embodiment, the siRNA is a nucleic acid that can form a double stranded RNA with the ability to reduce or inhibit expression of a gene or target gene. Each complementary sequence of the double stranded siRNA is about 15-50 nucleotides in length, and the double stranded siRNA is about 15-50 base pairs in length. A small hairpin RNA (shRNA) is also contemplated for use. The shRNA is an antisense strand of about 19 to about 25 nucleotides followed by a short nucleotide loop (approximately 5 to 9 nt) followed by the analogous sense strand. In an embodiment, an RNAi is a microRNA or miRNA, endogenous RNAs, some of which are known to regulate the expression of protein-coding genes at the posttranscriptional level. See, e.g., Lim et al., Science 299, 1540 (2003), Lee and Ambros, Science, 294, 862 (2001), Lau et al., Science 294, 858-861 (2001), Lagos-Quintana et al., RNA, 9, 175-179 (2003).

Different criteria are available for selecting the nucleic acid for use and may comprise scanning the mRNA sequence of the target, and may include empiric determination in accordance with, for example, Sui G et al., Proc. Natl. Acad. Sci. USA 99:5515-20 (2002). In some embodiments, criteria may further include confirmation the sequence lacks significant sequence homology with other genes as analyzed by BLAST search. Additional approaches may comprise any accessible site in endogenous mRNA that can be targeted for degradation by synthetic oligodeoxyribonucleotide/RNase H method (see, e.g., Lee N S et al., Nature Biotechnol. 20:500-05 (2002)). RNAi treatment may comprise miRNA or siRNA, or a pre-miRNA which is processed by Dicer to form a miRNA. The RNAi may also comprise a dsRNA or shRNA which is processed by Dicer to form a siRNA. The polynucleotides may comprise one or more modifications to suppress innate immune activation, enhance activity and specificity, and reduce off-target induced toxicity. Example teachings can be found, for example at Provost et al., E.M.B.O. J., 2002 Nov. 1; 21 (21): 5864-5874; Tabara et al., Cell 2002 Jun. 28; 109 (7): 861-71; Martinez et al., Cell 2002 Sep. 6; 110 (5): 563; Hutvagner & Zamore, Science 2002, 297:2056. In certain embodiments, a single-stranded RNAi agent disclosed herein can comprise substitutions, or modifications, including chemically modified nucleotides, and non-nucleotides which may include incorporation in the backbone, sugars, bases, or nucleosides. In an example, siRNA may comprise dual ribose modifications, including 2′,4′- and 2′,5′-modifications, 5′-E/Z-vinylphosphonate, and northern methanocarbacyclic (NMC) modifications. See, Gangopadhyay, RNA Biol. 2022 January; 19 (1): 452-467; doi: 10.1080/15476286.2022.2052641. The use of substituted or modified single-stranded RNAi agents can be designed to have an increased half-life in a subject. Furthermore, certain substitutions or modifications can be used to improve the bioavailability of single-stranded RNAi agents by targeting particular cells or tissues or improving cellular uptake of the single-stranded RNAi agents.

The RNAi molecule may decrease the mRNA level in a cell for a target gene by at least about 5%, about 10%, about 20%, about 30%, about 40%, about 50%, about 60%, about 70%, about 80%, about 90%, about 95%, about 99%, or about 100% of the mRNA level found in the cell without the presence of the miRNA or RNA interference molecule.

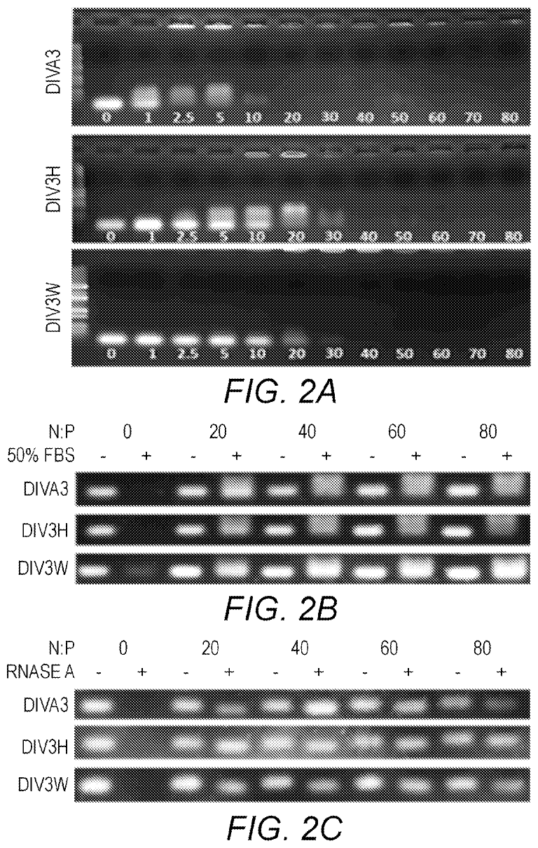

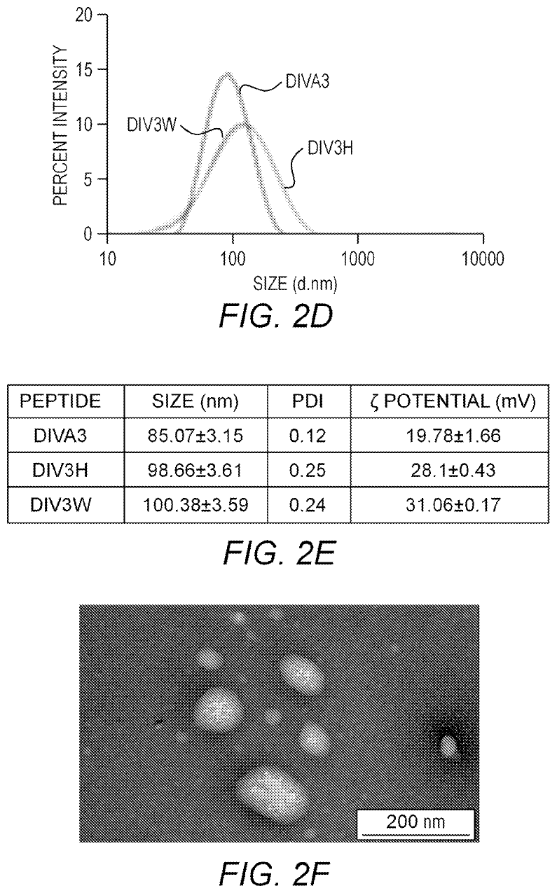

General design considerations for siRNAs, including potential chemical modifications in the siRNA are known in the art. See, generally, Hu, et al., Therapeutic siRNA: state of the art. Sig Transduct Target Ther 5, 101 (2020); doi: 10.1038/s41392-020-0207-x., see, e.g., FIGS. 2 and 3, specifically for its teachings of modifications. Exemplary FDA approved siRNA include patisiran, givosiran, lumasiran, and inclisiran which are approved for rare metabolic disorders. Additional example siRNAs are found in Hu et al. at Table 1, specifically incorporated by reference herein. In some embodiments, the siRNA are designed for an oncogene. In some embodiments, the siRNA inhibits the expression of an oncogene such as CSNK2A1. Exemplary human genes of CSNK2A1 include those described in Table 1 below, which can be used for design of siRNAs. In an aspect, the siRNA is designed for CSNK2A1 targeting exon 9, 10 or 11.

TABLE 1
CSNK2A1 sequences

| Description | Reference Sequence |
|---|---|
| Homo sapiens casein kinase 2 alpha 1 | NM_001895.4 |
| (CSNK2A1), transcript variant 2, mRNA | |
| Homo sapiens casein kinase 2 alpha 1 | NM_177559.3 |
| (CSNK2A1), transcript variant 1, mRNA | |
| Homo sapiens casein kinase 2 alpha 1 | NM_177560.3 |
| (CSNK2A1), transcript variant 3, mRNA | |
| Homo sapiens casein kinase 2 alpha 1 | NM_001362770.2 |
| (CSNK2A1), transcript variant 4, mRNA | |
| Homo sapiens casein kinase 2 alpha 1 | NM_001362771.2 |
| (CSNK2A1), transcript variant 5, mRNA | |

In some embodiments, the siRNA is specific for human epidermal growth factor receptor type 2 (HER2; ErbB2/neu), a member of the HER family, which is a transmembrane receptor tyrosine kinase. HER2 is known as an oncogenic driver of human breast and ovarian cancer. Example siRNAs targeting the Erb-b2 receptor have been interrogated, see, e.g., Oncotarget. 2016 Mar. 22; 7 (12): 14727-14741; doi: 10.18632/oncotarget.7409, incorporated herein by reference in its entirety, and specifically incorporated for Table SI siRNA sequences for Erb2, including d75 CACGUUUGAGUCCAUGCCCAA (SEQ ID NO:8) and d4 AUGGAGACCCGCUGAACAA (SEQ ID NO:9). Exemplary human genes of HER2 include those described in Table 2 below, which can be used for design of siRNA. In an aspect, the siRNA is designed for HER2 targeting exon 4, 7, or 8.

TABLE 2
HER2 Sequences

| Description | Reference Sequence |
|---|---|
| Homo sapiens erb-b2 receptor tyrosine kinase | NM_001005862.3 |
| 2 (ERBB2), transcript variant 2, mRNA | |
| Homo sapiens erb-b2 receptor tyrosine kinase | NM_001289936.2 |
| 2 (ERBB2), transcript variant 3, mRNA | |
| Homo sapiens erb-b2 receptor tyrosine kinase | NM_001289937.2 |
| 2 (ERBB2), transcript variant 4, mRNA | |
| Homo sapiens erb-b2 receptor tyrosine kinase | NM_001289938.2 |
| 2 (ERBB2), transcript variant 5, mRNA | |
| Homo sapiens erb-b2 receptor tyrosine kinase | NM_004448.4 |
| 2 (ERBB2), transcript variant 1, mRNA | |
| Homo sapiens erb-b2 receptor tyrosine kinase | NR_110535.2 |
| 2 (ERBB2), transcript variant 6, non-coding | |
| RNA | |

Commercially designed siRNAs, as well as siRNAs designed using design tools available from publicly available sources such as GenScript, Eurofins Genomics' siRNA design tool, and InvivoGen may be used. Design principles for the design of siRNA include those taught in Tuschl et al.: Targeted mRNA degradation by double-stranded RNA in vitro. Genes Dev 1999, 13 (24): 3191-7. doi: 10.1101/gad.13.24.3191; Reynolds et al., Rational siRNA design for RNA interference. Nat Biotechnol 22:326-330, 2004; Ui-Tei et al, Guidelines for the selection of highly effective siRNA sequences for mammalian and chick RNA interference. Nucleic Acids Res 32:936-948, 2004; and Vert et al., An accurate and interpretable model for siRNA efficacy prediction. BMC Bioinformatics 7:520, 2006.

Examples of a chemotherapeutic agent include without limitation: alkylating agents (e.g., which may include doxorubicin, cyclophosphamide, estramustine, carmustine, mitomycin, bleomycin and the like); antimetabolites (e.g., which may include 5-fluoro-uracil, capecitabine, gemcitabine, nelarabine, fludarabine, methotrexate and the like); platinating agents (e.g., which may include cisplatin, oxaliplatin, carboplatin and the like); topoisomerase inhibitors (e.g., which may include topotecan, irinotecan, etoposide and the like); tubulin agents (e.g., which may include paclitaxel, docetaxel, vinorelbine, vinblastine, vincristine, other taxanes, epothilones, and the like); signaling inhibitors (e.g., kinase inhibitors, antibodies, farnesyltransferase inhibitors, and the like); and other chemotherapeutic agents (e.g., tamoxifen, anti-mitotic agents such as polo-like kinase inhibitors or aurora kinase inhibitors, and the like).

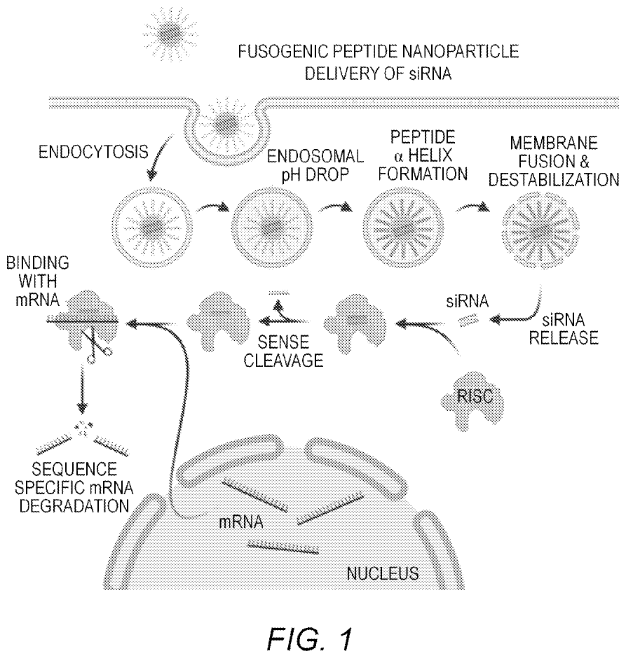

The cargo can be a gene editing system. Accordingly, methods of delivering a gene editing system-fusogenic peptide to a cell are provided. In an embodiment, methods of delivering a gene editng system for modulating a target gene are provided, comprising contacting a cell with a composition of the present invention comprising a cargo specific for the target polynucleotide (e.g., gene of interest), thereby modulating the expression of the gene in the cell. Gene editing systems that can be utilized as cargo in the present invention may comprise a CRISPR system, a zinc finger nuclease system, a meganuclease, or a TALEN system. A CRISPR-Cas system can comprise a Class 1 or Class 2 CRISPR-Cas system, which may comprise a guide sequence engineered to specifically bind a polynucleotide of interest and a Cas protein(s). As such, a ribonucleoprotein comprised of a Cas protein and a guide polynucleotide can be delivered. The CRISPR-Cas system that can be used to modify a target polynucleotide of the present invention described herein can be a Class 1 CRISPR-Cas system. Class 1 CRISPR-Cas systems are divided into types I, II, and IV. Makarova et al. 2020. Nat. Rev. 18:67-83., particularly as described in FIG. 1. Type I CRISPR-Cas systems include Types I-A, I-B, I-C, I-D, I-E, I-F1, I-F2, I-F3, and IG; Type III CRISPR-Cas systems can be Types III-A, III-B, III-C, III-D, III-E, and III-F; which can contain a Cas10 that can include an RNA recognition motif called Palm and a cyclase domain that can cleave polynucleotides; Type IV CRISPR-Cas systems include Types IV-A, IV-B, and IV-C. Class 2 systems comprise a single, large, multi-domain effector protein and can be a Type II, Type V, or Type VI system, which are described in Makarova et al. “Evolutionary classification of CRISPR-Cas systems: a burst of class 2 and derived variants” Nature Reviews Microbiology, 18:67-81 (February 2020), incorporated herein by reference. Class 2, Type II systems include II-A, II-B, II-C1, and II-C2; Type V systems include V-A, V-B1, V-B2, V-C, V-D, V-E, V-F1, V-F1 (V-U3), V-F2, V-F3, V-G, V-H, V-I, V-K (V-U5), V-U1, V-U2, and V-U4. Class 2, Type VI systems include VI-A, VI-B1, VI-B2, VI-C, and VI-D. Design of guides for targeting a nucleic acid for modification is known in the art, see, e.g., IDTdna.com and Synthego.com for guidance on custom guide RNAs. Reduction of off-target effects can be tailored using programs such as GUIDE-seq for the design of guide sequences for a desired target. See, e.g., Malinin, et al., Nature Protocols, 16, 5592-5615 (2012). Example targets can be designed for HER2 or CSNK2A1, for example, using the sequences as described in Tables 1 and 2. CRISPR-Cas systems that comprise a catalytically impaired Cas protein (e.g., Cas9) that is fused to an engineered reverse transcriptase, and a prime editing guide RNA (pegRNA) specific for a target polynucleotide that further encodes a desired edit for genome editing, (Anzalone et al. Nature 2019). These prime editing systems can be used for insertions, deletions and point mutations. Similarly, Adenine base editors (ABEs) to perform targeted A·T-to-G.C base pair conversion and Cytosine base editors (CBEs) to convert C·G to T·A have been be utilized with and without CRISPR-Cas systems for gene editing and can be utilized as cargo with the fusogenic peptides of the present invention. See, e.g., Komor et al., Nature 533, 420-424 (2016), Gaudelli et al., Nature 551, 464-471 (2017), Mok et al., Nature 583, 631-637 (2020); Koblan et al., Nature. 589, 608-614 (2021), Anzalone et al., Nature Biotechnology, 40, 731-740 (2022).

TALEN based gene editing is also contemplated and can be used in in vivo applications. See, S Becker, J Boch-Gene and Genome Editing, 2021. Zinc finger nuclease editing can also be utilized, and further modified to ensure high-precision gene editing. See, e.g., Conway et al., Molecular Therapy, 27:4, 10, 866-877 (2019); Paschon et al. Nature Comm, 10:1133 (2019). Similarly, editing can be made by meganucleases, characterized by a large recognition site of 12 to 40 base pairs of a double-stranded DNA sequence. See, e.g., U.S. Pat. Nos. 8,119,381, 10,273,524. Gene editing tools are well known in the art, with advantages and comparison of the tools that can be considered for the desired application. See, Rahim et al., Int'l J. of Innovative Science and Research Tech., 6:8 (2021), incorporated herein by reference.

Transposases may also be used as cargo in the compositions and methods of the present invention, including with gene editing systems described herein. Transposases include those comprising RNase H-like nuclease domains, such as Tn5, MuA, Mos1, Hermes, Serine and Tyrosine recombinases, including CTnDOT, Tn916, IS607 and TnpX, transposases comprising an HUH domain, including TnpA of IS91 or ISHp608, and helitron transposases, which can be as detailed in International Patent Publication WO2022056309, page 26, line 26-page 27, line 17, specifically incorporated by reference. See also nuclease guided transposase systems as described in WO2022150651 (DNA nuclease guided Transposase systems, Tn7-like transposition proteins with a Cas12k protein), WO2022147321 (Type I-B CRISPR Associated Transposase systems), WO2022076830 (Type I CRISPR Associated transposase systems), WO2021257997 (CAST); Li, et al., Int. J. Mol. Sci. 2020, 21 (21), 8329; doi: 10.3390/ijms21218329 (Tn5 transposase in applied genomic research).

The nanoparticle complex including the fusogenic peptide and a cargo can be designed to optimize complexation of the fusogenic peptide and the cargo. In an embodiment, the nanoparticle complex optimizes a ratio of N, the positively charged amine content of the fusogenic peptide, and the negatively charged content of the cargo. In some embodiments, the nanoparticle complex has a N:P ratio from about 20:1 to about 80:1. In some embodiments, the N:P molar ratio can be adjusted to optimize siRNA uptake for the fusogenic peptide complexes while still allowing for complexation of the fusogenic peptide and the cargo, as further described in the working examples.

### Methods of Using the Fusogenic Peptides

One aspect of the invention relates to a method of delivering cargo to a cell, comprising contacting the cell with an effective amount of a fusogenic peptide with a cargo, e.g., a complex, thereby delivering the cargo to the cell.

An aspect of the invention related to a method of increasing endosomal escape of a cargo delivered to the cell, comprising delivering the cargo as a nanoparticle complex comprising the fusogenic peptide and a cargo to the cell.

Another aspect of the invention relates to a method of inhibiting expression of a gene in a cell, comprising contacting the cell with an effective amount of a fusogenic peptide with a cargo such as, for example, siRNA that targets the gene, e.g., a complex, thereby inhibiting expression of the gene in the cell.

Methods may comprise modulating expression of a gene in a cell comprising contacting the cell with an effective amount of a fusogenic peptide with a cargo such as, for example, a gene editing system specific for a polynucleotide of interest (e.g., target sequence), as a nanoparticle complex, thereby modifying a polynucleotide of interest that modulates expression of a gene. In an embodiment, the polynucleotide of interest is targeted by a guide molecule (e.g., guide RNA) of the CRISPR-Cas system. In an embodiment, the gene editing system modulates the target system via deletion or insertion at a target polynucleotide. In an embodiment, the target is an oncogene, e.g., HER2 or CSNK2A1.

Another aspect of the invention relates to a method of treating, preventing, and/or delaying progression or onset of a disease or condition in a subject in need thereof, comprising delivering to the subject a therapeutically effective amount of a fusogenic peptide with a cargo, e.g., a complex, wherein the disease or condition is treatable by the cargo present in the pharmaceutical composition, thereby treating, preventing, and/or delaying progression of a disease or condition in the subject.

In some embodiments, the method comprises administration of a therapeutically effective amount of a fusogenic peptide with a cargo that targets a gene encoding a protein whose expression is associated with a disease or condition. In other embodiments, the administration of a therapeutically effective amount of a fusogenic polypeptide with a cargo inhibits the progression of cancer (e.g., ovarian or cervical cancer). In other embodiments, the administration of a therapeutically effective amount of a fusogenic polypeptide with a cargo reverses an existing cancer. In other embodiments, the administration of a therapeutically effective amount of a fusogenic polypeptide with a cargo accelerates the resolution of cancer.

Another aspect of the invention relates to a method of inhibiting expression of a human CK2α protein in a cell comprising contacting the cell with a therapeutically effective amount of a fusogenic peptide with a cargo, e.g., a complex, wherein the cargo is an siRNA targeting the CSNK2A1 gene, thereby inhibiting expression of the human CK2α protein in the cell. In another aspect, the invention further encompasses a method of treating cancer, inhibiting cancer growth, and/or decreasing the number of cancerous cells present in a subject in need thereof, comprising administering to the subject a therapeutically effective amount of a complex that has cargo with anti-cancer activity, thereby treating cancer, inhibiting cancer growth, and/or decreasing the number of cancerous cells present in the subject. For example, the complex may include cargo that suppresses the expression of an oncogene such as CSNK2A1.

Another aspect of the invention relates to a method of treating cancer in a subject in need thereof, wherein the cancer expresses or overexpresses a human CK2α protein, comprising delivering to the subject a therapeutically effective amount of a fusogenic peptide with a cargo, e.g., a complex, wherein the cargo is an siRNA targeting the CSNK2A1 gene, thereby treating cancer in the subject.

Another aspect of the invention relates to a method of treating cancer in a subject in need thereof, wherein the cancer expresses a human HER2 protein comprising delivering to the subject a therapeutically effective amount of a fusogenic peptide with a cargo, e.g., a complex, wherein the cargo is an siRNA targeting the HER2 gene, thereby treating cancer in the subject.

### Subjects, Pharmaceutical Formulations, and Modes of Administration

Fusogenic peptides and complexes according to the present invention find use in both veterinary and medical applications. Suitable subjects include both avians and mammals. The term “avian” as used herein includes, but is not limited to, chickens, ducks, geese, quail, turkeys, pheasant, parrots, parakeets, and the like. The term “mammal” as used herein includes, but is not limited to, humans, non-human primates, bovines, ovines, caprines, equines, felines, canines, lagomorphs, etc. Human subjects include neonates, infants, juveniles and adults.

In particular embodiments, the present invention provides a pharmaceutical composition comprising complexes, the complex comprising the fusogenic peptide and cargo of the invention, in a pharmaceutically acceptable carrier and, optionally, other medicinal agents, pharmaceutical agents, stabilizing agents, buffers, carriers, adjuvants, diluents, etc. For injection, the carrier will typically be a liquid. For other methods of administration, the carrier may be either solid or liquid. For inhalation administration, the carrier will be respirable, and optionally can be in solid or liquid particulate form.

By “pharmaceutically acceptable” it is meant a material that is not toxic or otherwise undesirable, i.e., the material may be administered to a subject without causing any undesirable biological effects.

The complexes of the invention can be formulated for administration in a pharmaceutical carrier in accordance with known techniques. See, e.g., Remington, The Science And Practice of Pharmacy (23^{rd} Ed. 2020). In the manufacture of a pharmaceutical formulation according to the invention, the complexes are typically admixed with, inter alia, an acceptable carrier. The carrier can be a solid or a liquid, or both, and may be formulated with the complexes as a unit-dose formulation, for example, a single use vial or a metered dose inhaler, which can contain from 0.01 or 0.5% to 95% or 99% by weight of the complex. One or more complexes, which may be the same or different (e.g., different cargoes and/or different targeting peptides), can be incorporated in the formulations of the invention, which can be prepared by any of the well-known techniques of pharmacy.

Another aspect of the invention relates to a kit comprising the fusogenic peptide, cargo and/or complex of the invention and useful for carrying out the methods of the invention. The kit may further comprise additional reagents for carrying out the methods (e.g., buffers, containers, additional therapeutic agents) as well as instructions.

One aspect of the present invention is a method of contacting a complex comprising a fusogenic peptide and cargo of the invention to a cell in vitro. The complex may be contacted with the cells at the appropriate concentration according to standard methods suitable for the particular target cells. Concentrations of the complex to administer can vary, depending upon the target cell type and number, and can be determined by those of skill in the art without undue experimentation. In representative embodiments, complexes at a concentration of at least about 10, 100, or 1,000 ng/mL, are contacted with the cell.

The cell(s) with which the complex is contacted can be of any type. Moreover, the cell can be from any species of origin, as indicated above.

The complex can be contacted with cells in vitro for the purpose of administering the modified cell to a subject. In particular embodiments, the cells have been removed from a subject, the complex is contacted therewith, and the cells are then administered back into the subject. Methods of removing cells from subject for manipulation ex vivo, followed by introduction back into the subject are known in the art (see, e.g., U.S. Pat. No. 5,399,346). In particular embodiments, the cells with the complex are administered to the subject in a treatment effective or prevention effective amount in combination with a pharmaceutical carrier.

In some embodiments, the complex is delivered to a cell in vitro or ex vivo by contacting the cell with an effective amount of the complex, thereby delivering the complex to the cell. In some embodiments, the cells are then transplanted to a subject in need thereof.

A further aspect of the invention is a method of administering the complex to subjects. Administration of the complex according to the present invention to a human subject or an animal in need thereof can be by any means known in the art. Optionally, the complex is delivered in a treatment effective or prevention effective dose in a pharmaceutically acceptable carrier. In some embodiments, the methods treat or prevent a disease, disorder, or condition by introducing a cargo, thereby modulating the expression of a gene. An example application includes cancer treatments, infection disease therapies, and gene editing. The administration of a nanoparticle comprising a cargo may increase or enhance expression or reduce or silence gene expression.

Dosages of the complex to be administered to a subject depend upon the mode of administration, the disease or condition to be treated and/or prevented, the individual subject's condition, and the like, and can be determined in a routine manner. Exemplary doses for achieving therapeutic effects are doses that achieve intracellular concentrations of at least about 10, 100, and 1,000 ng/mL. The amount of nanoparticle to administer can vary, depending upon the target cell type and number, and the particular nanoparticle and cargo, and can be determined by those of skill in the art without undue experimentation.

In particular embodiments, more than one administration (e.g., two, three, four or more administrations) may be employed to achieve the desired level of cargo in the cells or the desired level of gene expression (including increased or decreased levels) over a period of various intervals, e.g., daily, weekly, monthly, yearly, etc.

In particular embodiments, a complex according to the present invention is administered to the subject to treat, inhibit, and/or reverse cancer.

In some embodiments, the complex is administered to the subject by injection. In other embodiments, the complex is administered to the subject orally or topically. In the methods of the invention, the subject may be one has been diagnosed with cancer or is suspected of having cancer. Exemplary modes of administration include oral, rectal, transmucosal, topical, intranasal, inhalation (e.g., via an aerosol), buccal (e.g., sublingual), vaginal, intrathecal, intraocular, transdermal, in utero (or in ovo), parenteral (e.g., intravenous, subcutaneous, intradermal, intramuscular [including administration to skeletal, diaphragm and/or cardiac muscle], intradermal, intrapleural, intracerebral, and intraarticular), topical (e.g., to both skin and mucosal surfaces, including airway surfaces, and transdermal administration), intro-lymphatic, and the like, as well as direct tissue or organ injection (e.g., to liver, skeletal muscle, cardiac muscle, diaphragm muscle or kidney). The most suitable route in any given case will depend on the nature and severity of the condition being treated.

In one embodiment, the complex of the invention is administered directly to a subject. Generally, the compositions of the invention will be suspended in a pharmaceutically-acceptable carrier (e.g., physiological saline) and administered orally or by intravenous infusion, or administered subcutaneously, intramuscularly, intrathecally, intraperitoneally, intrarectally, intravaginally, intranasally, intragastrically, intratracheally, or intrapulmonarily. In another embodiment, the intratracheal or intrapulmonary delivery can be accomplished using a standard nebulizer, jet nebulizer, wire mesh nebulizer, dry powder inhaler, or metered dose inhaler. They can be delivered directly to the site of the disease or disorder, such as lungs, kidney, or intestines. The dosage required depends on the choice of the route of administration; the nature of the formulation; the nature of the patient's illness; the subject's size, weight, surface area, age, and sex; other drugs being administered; and the judgment of the attending physician. Suitable dosages are in the range of 0.01-100.0 μg/kg. Wide variations in the needed dosage are to be expected in view of the variety of polypeptides and cargo available and the differing efficiencies of various routes of administration. For example, oral administration would be expected to require higher dosages than administration by i.v. injection. Variations in these dosage levels can be adjusted using standard empirical routines for optimization as is well understood in the art. Administrations can be single or multiple (e.g., 2-, 3-, 4-, 6-, 8-, 10-; 20-, 50-, 100-, 150-, or more fold). Encapsulation of the polypeptides and cargo in a suitable delivery vehicle (e.g., polymeric microparticles or implantable devices) may increase the efficiency of delivery, particularly for oral delivery.

According to certain embodiments, the complex can be targeted to specific cells or tissues in vivo. Targeting delivery vehicles, including liposomes and targeted systems are known in the art. For example, a liposome can be directed to a particular target cell or tissue by using a targeting agent, such as an antibody, soluble receptor or ligand, incorporated with the liposome, to target a particular cell or tissue to which the targeting molecule can bind. Targeting liposomes are described, for example, in Ho et al., Biochemistry 25:5500 (1986); Ho et al., J. Biol. Chem. 262:13979 (1987); Ho et al., J. Biol. Chem. 262:13973 (1987); and U.S. Pat. No. 4,957,735 to Huang et al., each of which is incorporated herein by reference in its entirety).

An additional aspect of the invention relates to a dosage delivery device comprising the pharmaceutical composition. In some embodiments, the dosage delivery device is an inhaler for delivery of the composition to the airways of a subject, e.g., by oral and/or nasal inhalation.

Injectables can be prepared in conventional forms, either as liquid solutions or suspensions, solid forms suitable for solution or suspension in liquid prior to injection, or as emulsions. Alternatively, one may administer the complex of the invention in a local manner, for example, in a depot or sustained-release formulation. Further, the complex can be delivered adhered to a surgically implantable matrix.

Non-limiting examples of formulations of the invention include those suitable for intravenous administration of the nanoparticles. Oral, rectal, buccal (e.g., sub-lingual), vaginal, parenteral (e.g., subcutaneous, intramuscular including skeletal muscle, cardiac muscle, diaphragm muscle and smooth muscle, intradermal, intravenous, intraperitoneal), topical (i.e., both skin and mucosal surfaces, including airway surfaces), intranasal, transdermal, intraarticular, intracranial, intrathecal, and inhalation administration, administration to the liver by intraportal delivery, as well as direct organ injection (e.g., into the liver, into a limb, into the brain or spinal cord for delivery to the central nervous system, into the pancreas, or into a tumor or the tissue surrounding a tumor) are also envisioned. The most suitable route in any given case will depend on the nature and severity of the condition being treated and on the nature of the particular complex which is being used. In some embodiments, it may be desirable to deliver the formulation locally to avoid any side effects associated with systemic administration. For example, local administration can be accomplished by direct injection at the desired treatment site, by introduction intravenously at a site near a desired treatment site (e.g., into a vessel that feeds a treatment site, or intramuscular administration with muscle specific promoters). In some embodiments, the formulation can be delivered locally to ischemic tissue.

For injection, the carrier will typically be a liquid, such as sterile pyrogen-free water, pyrogen-free phosphate-buffered saline solution, bacteriostatic water, or Cremophor EL [R] (BASF, Parsippany, N.J.). For other methods of administration, the carrier can be either solid or liquid.

For oral administration, the compound can be administered in solid dosage forms, such as capsules, tablets, and powders, or in liquid dosage forms, such as elixirs, syrups, and suspensions. Examples of additional inactive ingredients that can be added to provide desirable color, taste, stability, buffering capacity, dispersion or other known desirable features are red iron oxide, silica gel, sodium lauryl sulfate, titanium dioxide, edible white ink and the like. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain coloring and flavoring to increase patient acceptance.

Formulations suitable for buccal (sub-lingual) administration include lozenges comprising the compound in a flavored base, usually sucrose and acacia or tragacanth; and pastilles comprising the compound in an inert base such as gelatin and glycerin or sucrose and acacia.

Formulations of the present invention suitable for parenteral administration comprise sterile aqueous and non-aqueous injection solutions of the compound, which preparations are preferably isotonic with the blood of the intended recipient. These preparations can contain anti-oxidants, buffers, bacteriostats and solutes which render the formulation isotonic with the blood of the intended recipient. Aqueous and non-aqueous sterile suspensions can include suspending agents and thickening agents. The formulations can be presented in unit/dose or multi-dose containers, for example sealed ampoules and vials, and can be stored in a freeze-dried (lyophilized) condition requiring only the addition of the sterile liquid carrier, for example, saline or water-for-injection immediately prior to use.

Having described the present invention, the same will be explained in greater detail in the following examples, which are included herein for illustration purposes only, and which are not intended to be limiting to the invention.

### Example 1

A set of rationally designed fusogenic peptides were developed for complexation with siRNAs to be delivered into genetically relevant epithelial ovarian cancer cell lines, OVCAR3 and CAOV3. Each peptide was designed to maintain amphipathic properties and include a sequence of D-arginine residues responsible for siRNA complexation. The ability of three fusogenic peptides to electrostatically bind with siRNAs, protect siRNA cargo from serum degradation, and encourage endosomal escape and delivery of bioactive siRNAs was demonstrated. Specifically, the potential of CSNK2A1 as a gene target was examined in ovarian cancer via delivery of the novel fusogenic peptides complexed with siRNAs. CSNK2A1 is an oncogene found overexpressed in several cancer types, including ovarian cancer, and is responsible for increased growth and cell survivability in the presence of chemotherapeutic agents. The protein encoded by CSNK2A1, CK2a, plays a major role in cell cycle regulation, cell-cell communication, DNA repair activation, and apoptosis in response to external stimuli in lung adenocarcinoma, urothelial carcinoma, and pancreatic adenocarcinoma. Roles in ovarian cancer, however, have only been suggested through observation of upregulation of CK2α and combination treatment with chemotherapeutics including dasatinib, cisplatin, and gemcitabine. RNAi-based therapies alone that target CSNK2A1 have not been comprehensively studied; thus, the therapeutic efficacy of siCSNK2A1 monotherapy in ovarian cancer delivered using novel fusogenic peptides was assessed.

The knockdown of CK2α protein using the DIV3W peptide complexed with siRNA, targeting CSNK2A1, decreased cell migration and invasion in ovarian cancer. The DIV3W peptide encouraged high cellular uptake efficiency and significant gene and protein silencing. Cytotoxic effects from targeting CSNK2A1 were not observed. The reduction in cell migration and colonization demonstrates the therapeutic potential of CSNK2A1 as a gene target to reduce ovarian cancer aggressiveness. Furthermore, these data demonstrate the potential of the DIV3W peptide as a delivery system for RNAi-based therapy.

### Materials and Methods

Cell Culture. Ovarian epithelial cancer cell lines, OVCAR3, obtained from the American Type Culture Collection (ATCC, Manassas, VA), and CAOV3 (donated from Hollings Cancer Center, Charleston, SC) were cultured in McCoy's 5A culture medium and Dulbecco's Modified Eagles Medium (DMEM, ATCC), respectively. The cultures were supplemented with 10% fetal bovine serum (FBS, Corning, Corning, NY) and 1% penicillin/streptomycin (P/S) antibiotic solution (Corning). All cultures were incubated at 37° C. in 5% CO₂.

Peptide Synthesis. Peptides, DIVA3 (WEADIVADIVADIVAGGG-(d) RRRRRRRRR) (SEQ ID NO:1), DIV3H (WEADIVADIVHDIVADIVAGGG-(d) RRRRRRRRR) (SEQ ID NO: 2), and DIV3W (WEADIVADIVWDIVADIVAGGG-(d) RRRRRRRRR) (SEQ ID NO:3), were synthesized, purified (>95% purity), and analyzed via high performance liquid chromatography (HPLC) by Genscript (Genscript USA Inc., Piscataway, NJ). All formulations contained an end sequence of nine D-arginine residues with no terminal modifications. Lyophilized peptide formulations were resuspended in analytical grade dimethyl sulfoxide (DMSO) to create stock peptide solutions of 100 μM (10 mg/mL). Working solutions of 10 μM (1 mg/mL) were created using a 1:10 dilution of stock peptide in RNase-free water and all solutions were stored at −80° C.

Gel Shift Assays. DIVA3, DIV3H, and DIV3W (all three denoted as DIV3 (X)) peptides suspended in RNase-free water (10 μM), were thawed, and kept on ice prior to complexation with 10 μM of negative control siGENOME non-targeting siRNA #5 (siNT, GE Healthcare Dharmacon, Lafayette, CO) in RNase-free water and incubated for 20 minutes at room temperature to allow the formation of DIV3 (X)-siNT complexes. Various molar ratios of peptides were complexed with siNT at increasing N:P molar ratios to determine the minimum molar ratio necessary to completely complex free siRNAs using an electrophoretic mobility shift assay. To determine the ability of each peptide variant to protect siRNAs from degradation, DIV3 (X)-siNT complexes or siNT alone was left untreated or incubated with either 50% v/v FBS or 5 μg/mL RNase A at 37° C. for one hour. Next, complexes were dissociated using 6% sodium dodecyl sulfate (SDS). Samples were added to a 2% agarose gel and run at 100 V. Gels were stained with ethidium bromide, destained with Milli-Q water, and imaged using a ChemiDoc XRS imager (Bio-Rad, Hercules, CA).

Dynamic Light Scattering and Transmission Electron Microscopy. DIV3 (X)-siNT complexes were prepared at 2 mg/mL in RNase-free water. Hydrodynamic size and surface charge of the peptide/siRNA complexes were determined using a Nano ZS Zetasizer (Malvern Pananalytical, Malvern, UK). All data were recorded using Malvern's Zetasizer software.

To obtain transmission electron microscopy (TEM) images of the peptide-siRNA complexes, DIV3W-siNT complexes were prepared to a concentration of 1 mg/mL in RNase-free water. After a 20-minute incubation, TEM samples were prepared via drop casting onto a copper mesh grid. Following a 5-minute drying period, the remaining water was wicked away and the sample was background stained with 1% uranyl acetate solution (Electron Microscopy Sciences, Hatfield, PA). Samples were stored for 48 hours to allow for complete solution evaporation and sample crystallization. TEM was completed using a Hitachi HT7800 microscope (Hitachi, White Plains, NY) and imaged at 80,000× magnification.

Cytotoxicity. Cells were seeded at 10,000 cells per well with complete culture media in a 96-well plate and incubated overnight to allow the cells to attach. DIV3 (X) peptides were complexed with siNT at 40:1 to 80:1 N:P molar ratios. DIV3 (X)-siNT complexes were incubated with OVCAR3 or CAOV3 cells at a final concentration of 100 nM siRNA and 10% FBS for 24 hours. Media was then aspirated, cells were washed with 1× phosphate-buffered saline (PBS), and fresh media with 10% FBS was added to each well and incubated another 24 hours.

Viability was measured using an MTS Assay performed with CellTiter 96 AQueous One Solution Cell Proliferation Assay (Promega, Madison, WI). Assay reagent was added according to the manufacturer's protocol, and cells were incubated for 4 hours. Absorbance was read using a BioTek Synergy LX (BioTek, Winooksi, VT) plate reader at 490 nm. An MTS cell viability assay was also conducted as described for OVCAR3 and CAOV3 cells treated with DIV3W-siRNA complexes.

Cellular Uptake. OVCAR3 and CAOV3 cells were prepared at 50,000 cells per well in a 24-well plate. DY547 fluorescently labeled siNT (siNT-DY547, GE Healthcare Dharmacon) was complexed with the DIV3 (X) peptides in RNase free water at 40:1 to 80:1 N:P molar ratios and incubated with OVCAR3 or CAOV3 cells for 4 hours. Post-incubation, media was aspirated and cells were washed with 1× PBS. To counterstain the nuclei, SuperSignal NucBlue Hoechst stain (Thermo Fisher, Waltham, MA) was added to the cells for 30 minutes, followed by an additional 1×PBS wash. Subsequent imaging was performed using an EVOS FL Microscope (Thermo Fisher) at 10× magnification. For flow cytometric analysis, cells were detached, resuspended in 1× PBS, and analyzed using an Attune NXT Acoustic flow cytometer (Invitrogen, Carlsbad, CA) with the blue laser line and BL2 (574/26 nm) channel.

For analysis of endosomal escape, following the 4- and 8-hour treatments, OVCAR3 and CAOV3 cells were fixed with 4% paraformaldehyde in 1×PBS and permeated with 0.5% Triton X-100 in 1× PBS for 15 minutes at room temperature. Blocking was completed after washing three times with 1× PBS by incubating with 1% bovine serum albumin in 1× PBS for 30 minutes at room temperature. Primary antibodies for early endosome antigen-1 (rabbit anti-EEA1, Thermo Fisher) were diluted in 1× PBS (1:100) and incubated with cells for 1 hour at 4° C. in the dark. Following washing three times using 1×PBS, cells were incubated with Alexa Fluor 488 goat anti-rabbit secondary antibodies (Invitrogen) in a 1:100 dilution at room temperature for 1 hour. Cells were washed three more times with 1×PBS, stained with SuperSignal NucBlue Hoechst 33342, and imaged using an EVOS FL microscope at 10× magnification.

qRT-PCR. Cells were seeded at 50,000 cells per well and treated with siRNA targeting CSNK2A1 (siCSNK2A1 #s3638, Thermo Fisher Scientific) at increasing N:P ratios and incubated for a total of 48 hours in McCoys 5A/DMEM-10% FBS. Treated cells were washed with 1×PBS at 24 hours and fresh media was added. RNAisolation was completed using a Qiagen Mini-RNeasy Kit (Qiagen, Hilden, Germany) according to the manufacturer's protocol. RNA isolation quality and quantity were measured through the Gen5 protocol using a Take3 plate (BioTek). RNA was reverse-transcribed using the QuantiTect Reverse Transcription kit (Qiagen). Quantitative real-time PCR was completed with a QuantStudio 3 qPCR system (Applied Biosystems) using Applied Biosystems TaqMan FAST Master Mix (Thermo Fisher) and CSNK2A1 (ID #Hs00751002_s1) and 18S (ID #Hs99999901_s1) predesigned TaqMan probes, according to the manufacturer's protocol.

Western Blot. Cells were seeded at 100,000 cells per well with complete culture media in a 6-well plate and left to incubate overnight. Cells were washed and treated with DIV3 (X)-siRNA complexes as previously described. Following the 48-hour treatment, cells were washed with 1× PBS and lysed with cold radioimmunoprecipitation assay (RIPA) buffer supplemented with protease inhibitor cocktail (Thermo Fisher). Protein concentrations were determined via bicinchoninic assay according to the manufacturer's protocol and absorbance was read at 562 nm. Equivalent protein concentrations for each treatment condition were separated by SDS-PAGE on a BioRad Stain-Free 10% gel according to BioRad protocols and transferred to a Stain-Free PVDF membrane using a wet transfer system. The PVDF membrane was cut at appropriate molecular weight intervals consistent with protein weights, then analyzed and blocked for 1 hour with 5% non-fat dried milk solution in 1× Tris-HCl-buffered-saline-0.1% Tween 20 (TBST) at room temperature. Blots were probed at 4° C. overnight with rabbit monoclonal anti-CK2α antibody (1:1000, Invitrogen) or mouse monoclonal anti-β-actin (1:10,000, Sigma-Aldrich, St. Louis, MO). Membranes were washed five times with 1× TBST and incubated with goat anti-rabbit (1:1000, Thermo Fisher) or goat anti-mouse (1:10,000, Thermo Fisher) secondary antibodies for 1 hour at room temperature. Five additional washes with 1× TBST were performed and protein bands were detected with SuperSignal West Pico Plus Chemiluminescent Substrate (Thermo Fisher) and imaged using a BioRad ChemiDoc Imaging System.

Cell Migration and Colonization Analysis. Scratch migration was completed by seeding OVCAR3 or CAOV3 cells at 100,000 cells per well in a 12-well plate to achieve near 100% confluency. Following a 48-hour treatment with DIV3W-siCSNK2A1 or DIV3W-siNT complexes, a vertical and horizontal scratch was made in the cell monolayer using a 200 μL micropipette tip and edges were smoothed using a gentle wash with 1× PBS. Cells were imaged with an EVOS FL microscope at 4× magnification at 24-hour intervals at the cross intersections to ensure consistent imaging areas. Using image analysis software, measurements across the horizontal axis were taken for each image with wound closure percentages calculated at each time point using the equation below (Eq. 1) where ‘ti’ is the distance at any time point ‘i’ and ‘to’ is the distance at time 0.

$$Percent Wound Heal ( {t}_{i} ) = 100 - [ \frac{( {t}_{i} - {t}_{0} )}{{t}_{0}} \times 100 ] \% . \qquad Equation 1$$

$$Formula for calculating percent wound heal in the scratch wound assay . The distance at t = 0 , {t}_{0} , was deducted from each subsequent distance measurement , {t}_{i} , at t = i . Distance difference was normalized to {t}_{0} and subtracted from 100 to represent the percent distance closed from {t}_{0} .$$

Colonization of treated cells was determined using a clonogenic assay and quantified via absorbance at 490 nm. Cells were seeded at 50,000 cells per well in a 24-well plate and treated with DIV3W-siCSNK2A1 or DIV3W-siNT complexes as previously described. Cells were harvested, counted, and reseeded in a 6-well plate at a concentration of 2,500 cells per well. Cell growth was monitored for 14 days with media changes every two days. After the growth period, cells were washed with 1×PBS and stained with a 0.1% crystal violet solution for 30 minutes. Unbound crystal violet was washed with ddH₂O and plates were air-dried for 15 minutes and imaged with the ChemiDoc Imaging System. Recolonization was quantified by dissolving bound crystal violet in a 33% acetic acid solution and transferring 100 μL of each treatment group to a 96-well plate in triplicate. Absorbance was measured at 490 nm using a BioTek Synergy LX plate reader.

Statistical Analysis. All data are mean±SEM of three independent experiments. Statistical comparisons were carried out using GraphPad Prism 8.4.3. Comparisons between two independent groups were conducted using a Student's t-test. Three or more independent groups with one factor were analyzed using ANOVA and post-hoc Tukey comparisons. Three or more independent groups with two factors were analyzed using two-way ANOVA, also with post-hoc Tukey comparisons. Results were considered statistically significant where p≤0.05.

### Results

Characterization of DIVA3, DIV3H, and DIV3W peptides. Peptide design was inspired by the prominent fusogenic peptide, GALA, a synthetic peptide derived from the viral fusion sequence of the HA2 protein found in the influenza virus. Each amino acid was selected to formulate an amphipathic sequence repeat, consisting of hydrophilic and hydrophobic residues. The N-terminus includes a nona-d-arginine sequence to enable electrostatic complexion with siRNAs, which is separated from the amphipathic, fusogenic sequence at the C-terminus by a glycine linker. The three peptides, DIVA3, DIV3H, and DIV3W, were designed to follow these formulations, with an additional histidine residue and tryptophan residue present in DIV3H and DIV3W, respectively.

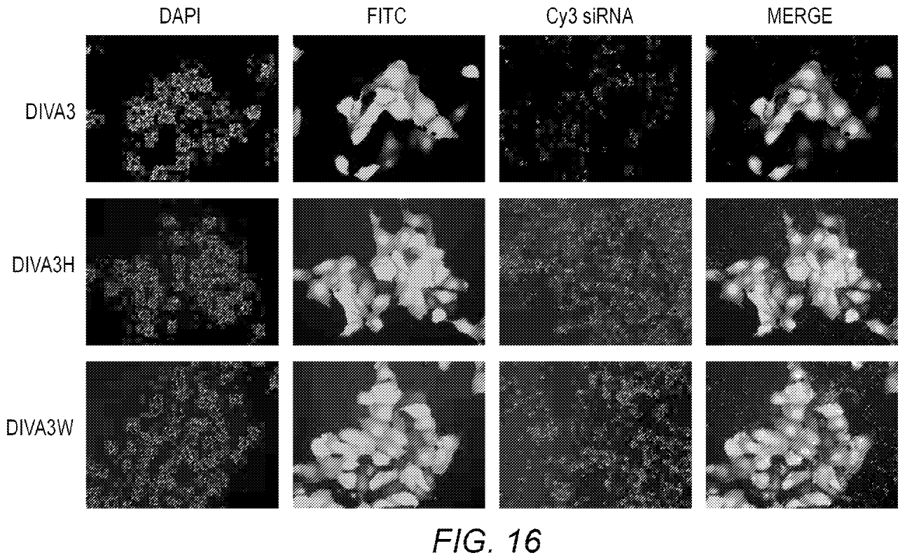

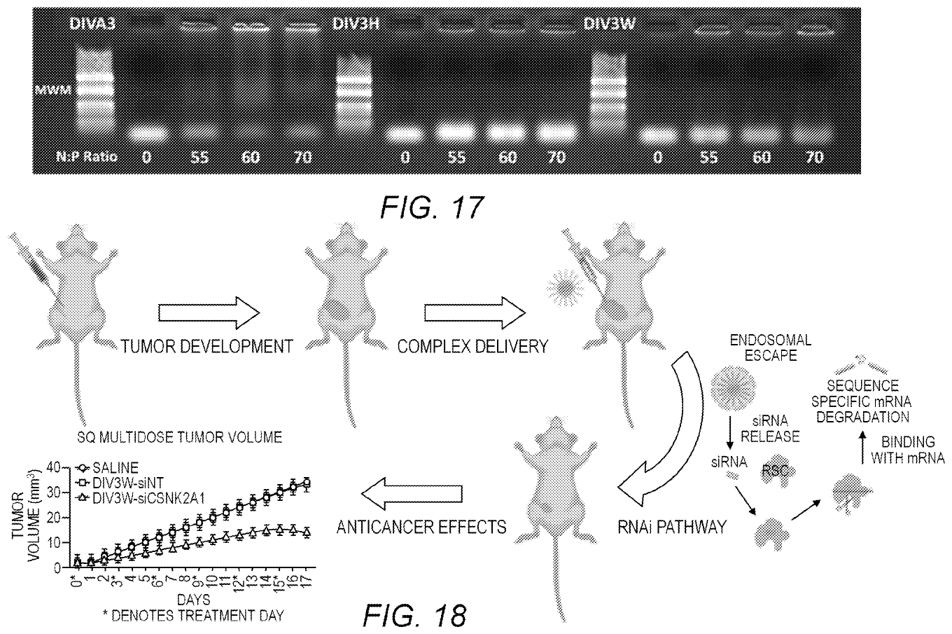

Agarose gel electrophoresis was used to determine the ability of DIVA3, DIV3H, and DIV3W (all three denoted as DIV3 (X)) peptides to completely complex free siRNAs and protect siRNAs from degradation. The DIVA3 peptide was able to completely complex free siRNAs at the lowest molar ratio of positively charged amine content in the peptide to negatively charged phosphate content in the siRNA (N:P), at 20:1. In contrast, a minimum molar ratio of 40:1 was required for complete complexation of DIV3H and DIV3W with siRNAs (FIG. 2A). However, when the arginine tail is truncated to only 5 arginine residues, FIG. 17 shows that the three truncated peptides of DIVA3, DIV3H, and DIV3W were unable to complex all the available siRNAs. To mimic physiological conditions and examine the ability of the peptides to protect siRNAs from degradation, DIV3 (X) peptides were complexed with non-targeting siRNA (siNT) at several N:P ratios and exposed to 50% serum (FIG. 2B) or RNase A (FIG. 2C) for 1 hour followed by agarose gel electrophoresis. White bands indicate intact siRNA and can be observed for DIV3W-siRNA complexes subjected to 50% serum at all N:P molar ratios. Reduction of intact siRNA signal was detected for DIVA3 and DIV3H complexes at 40:1 to 80:1 ratios incubated with 50% serum (FIG. 2B) and at 20:1 and 80:1 molar ratios of the DIVA3 complexes when incubated with RNase A (FIG. 2C). Minimal degradation, specifically in the DIV3W formulations after incubation in RNase A, indicates that the peptides protected siRNA from degradation (FIG. 2C).

Peptide molar ratios of 40:1, 60:1, and 80:1 were selected for subsequent experiments following confirmation of full siRNA complexation at each of these ratios for all three peptide variants. Each peptide/siRNA complex was characterized by dynamic light scattering to determine the nanoparticle size distribution and surface charge to ensure stability (FIGS. 2D-2E). All DIV3 (X)-siRNA complexes formed positively charged, monodisperse nanoparticles that were within the appropriate size range advantageous for passive targeting through the enhanced permeability and retention effect. DIV3W-siRNA and DIV3H-siRNA complexes were the largest, with a hydrodynamic diameter of 100.38±3.59 nm and 98.66±±3.62 nm, respectively, while DIVA3 formed complexes sized 85.07±3.15 nm (FIGS. 2D-2E). Transmission electron microscopy (TEM) of DIV3W-siRNA complexes (FIG. 2F) confirmed the formation of spherical nanoparticles.

DIV3 (X) peptides exhibit biocompatibility with ovarian cancer cells. To evaluate the effect of the peptides on cell viability, OVCAR3 and CAOV3 cells were treated with DIV3 (X) peptides alone at concentrations equivalent to 40:1, 60:1, and 80:1 molar ratios for each formulation. After a 48-hour treatment, none of the peptide formulations demonstrated any significant cytotoxicity compared to untreated cells (FIGS. 3A-3B). To examine biocompatibility with healthy cells, DIV3W was treated for 48 h on MCF10a breast epithelial cells where no cytotoxic effects were observed (FIG. 25). These results confirm the biocompatibility of the DIV3 (X) peptides and their potential for in vivo evaluation.

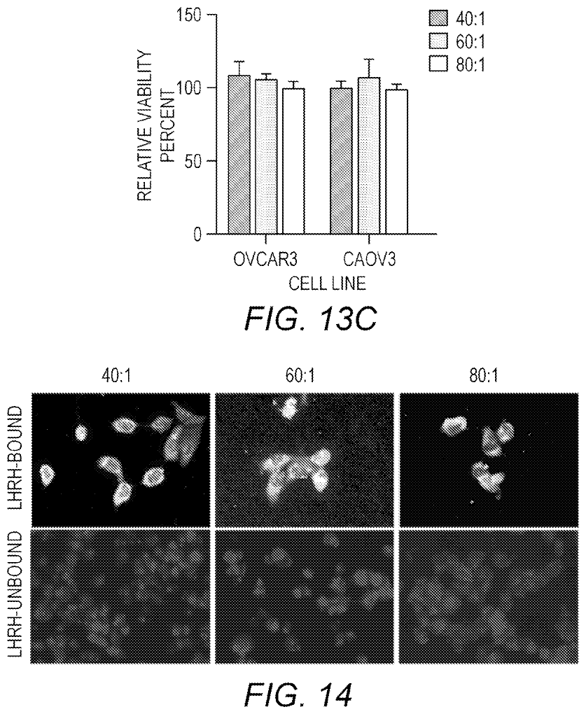

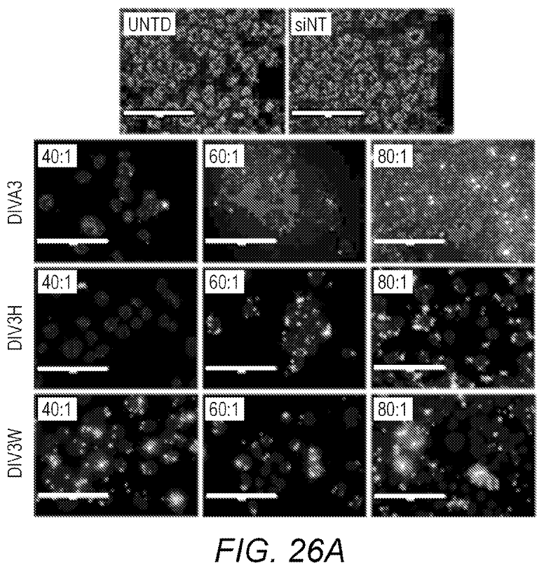

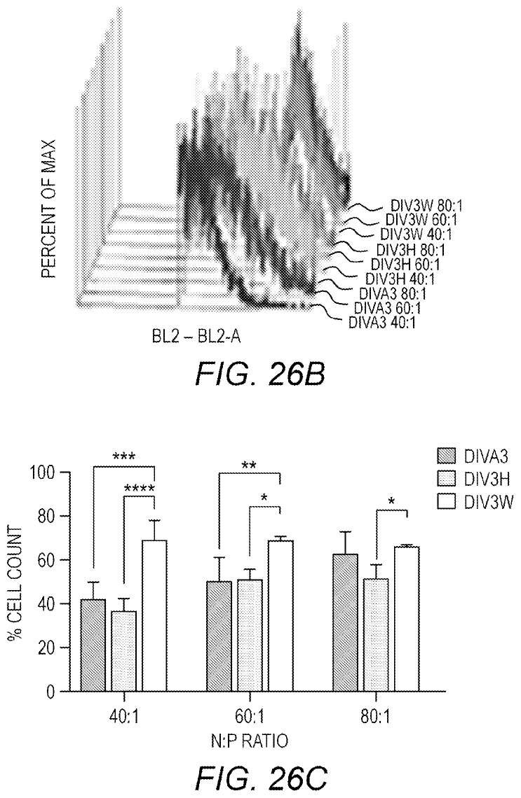

DIV3 (X) peptides enable siRNA uptake into ovarian cancer cells via endocytosis. A major barrier to siRNA delivery is inefficient cellular uptake. Through fluorescence microscopy and flow cytometry, the ability of the DIV3 (X) peptides to deliver fluorescently labeled siRNAs into ovarian cancer cells was examined. Fluorescence microscopy revealed that uptake of DY547 fluorescently labeled non-targeting siRNA (siNT-DY547) into OVCAR3 and CAOV3 cells increased with increasing N:P ratios across each peptide variant. Furthermore, siNT-DY547 uptake into ovarian cancer cells also increased with the use of DIV3W compared to DIVA3 and DIV3H (FIGS. 4A, 13A and 26A). Qualitative observations of siRNA uptake were confirmed with flow cytometry, which also demonstrated increasing uptake efficiency with increasing N:P ratios (FIGS. 4B, 13B and 26B). N:P molar ratio-dependent increases in siRNA uptake were apparent for all formulations, except DIV3W, which displayed high levels of uptake across all N:P ratios in OVCAR3 cells (FIG. 4C). DIV3W uptake efficiency was 70.0%, 69.7%, and 66.6% for 40:1, 60:1, and 80:1 molar ratios, respectively. DIV3H and DIVA3 peptides exhibited optimum cell uptake at an 80:1 ratio, at 52.1% and 63.6% efficiency, respectively (FIG. 4C). In CAOV3 cells, no statistically significant results were found between peptide variants, but uptake did increase with higher N:P ratios (FIG. 13C; FIG. 26C).

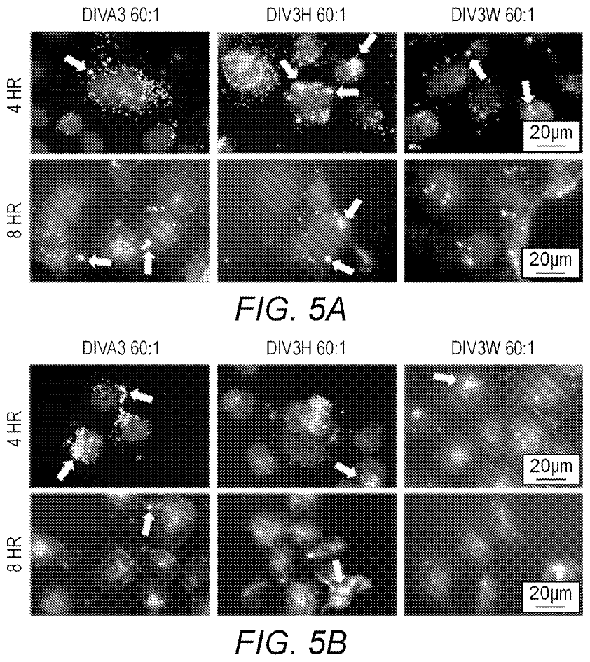

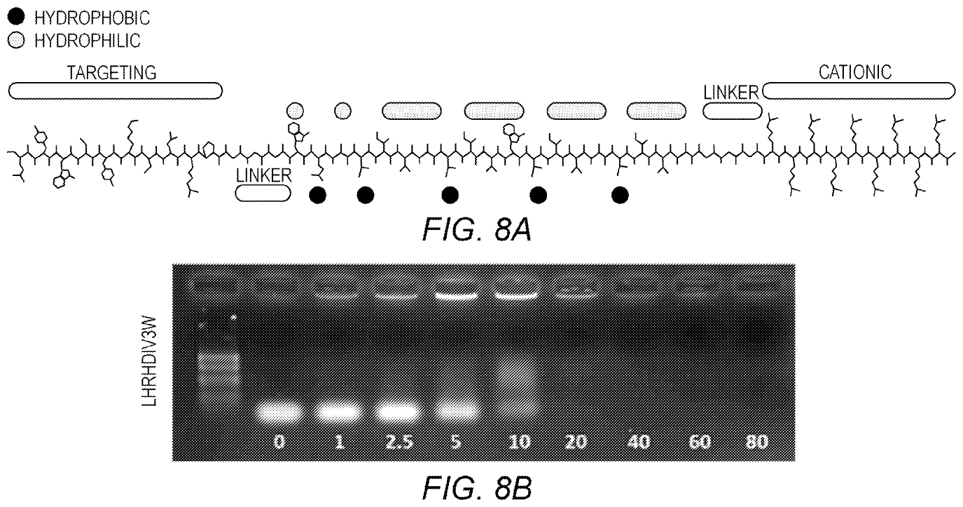

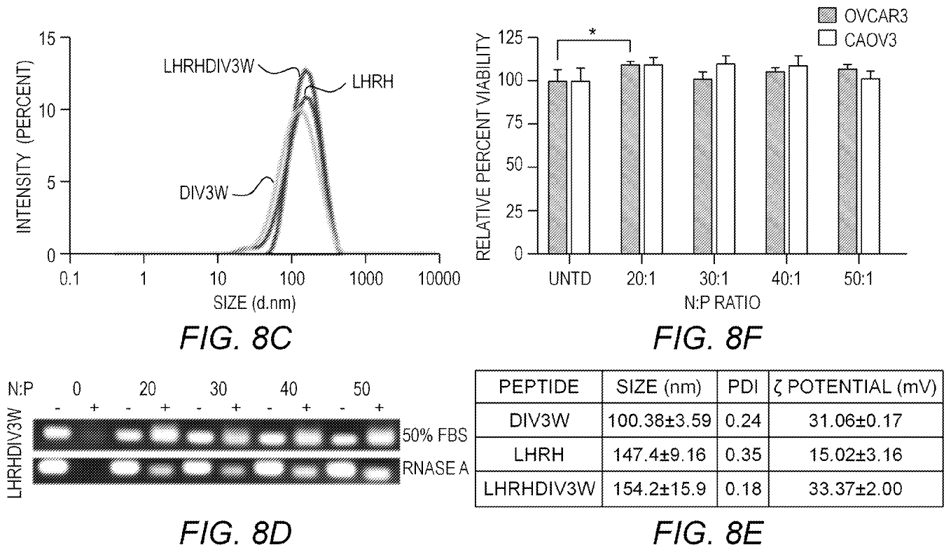

Another significant barrier to the delivery of endocytosed nanocarriers is endosomal entrapment, resulting in lysosomal trafficking and degradation of nanocarriers, which inhibit the bioactivity of the delivered siRNAs. The primary functional characteristic of the DIV3 (X) fusogenic peptides is their ability to overcome this barrier by mediating endosomal escape through fusion of exposed hydrophobic amino acids with the endosomal membrane following protonation of anionic residues. Each DIV3 (X) peptide was complexed with siNT-DY547 at a 60:1 molar ratio and incubated with OVCAR3 or CAOV3 cells for 4 (FIG. 5A) and 8 hours (FIG. 5B) prior to staining for early endosome antigen-1 (EEA1). Endosomal accumulation of siRNA indicates intracellular localization of the delivered complexes and was observed for all peptide complexes after 4 hours. However, colocalized signal of siNT-DY547 and EEA1 stain decreased after allowing the complexes to incubate for 8 hours before imaging, suggesting successful release of siRNA cargo from the endosome into the cytosol.

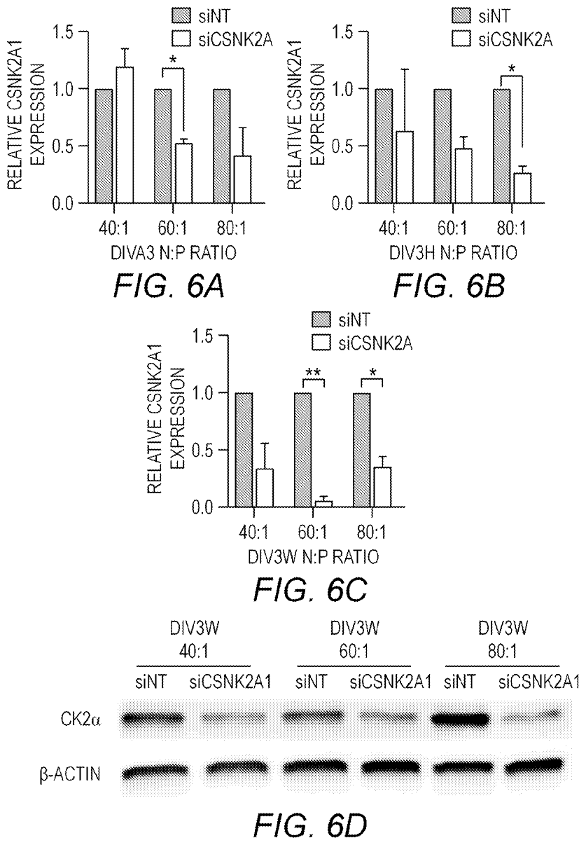

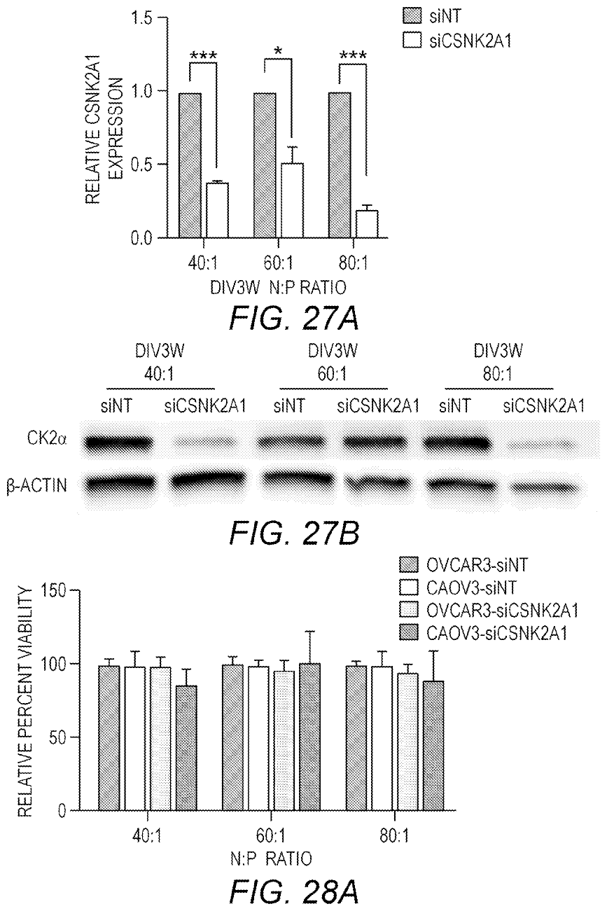

DIV3W-siCSNK2A1 complexes silence CSNK2A1 mRNA and CK2a. protein expression in ovarian cancer cells. Confirmation of endosomal escape of siRNAs mediated by the DIV3 (X) peptides was further confirmed through evaluation of siRNA bioactivity via gene silencing. Higher levels of gene silencing are associated with higher levels of cellular uptake, endosomal escape, and release of bioactive siRNAs. Gene silencing efficiency was determined by analyzing CSNK2A1 mRNA and CK2α protein levels in ovarian cancer cells treated with DIV3 (X) peptides complexed with siNT or siCSNK2A1 for 48 hours. DIVA3 (FIG. 6A) and DIV3H (FIG. 6B) complexes mediated statistically significant reduction of CSNK2A1 mRNA expression in OVCAR3 cells, but only at a 60:1 ratio in DIVA3 complexes and an 80:1 ratio in DIV3H complexes at 47% and 72% silencing efficacy, respectively (FIGS. 6E and 6F). DIV3W-siCSNK2A1 complexes significantly reduced CSNK2A1 mRNA expression in OVCAR3 cells compared to DIV3W-siNT by 95% and 65% at 60:1 and 80:1 molar ratios, respectively (FIG. 6C). Significant reduction of CSNK2A1 mRNA expression was also observed in CAOV3 cells when treated with DIV3W complexes at all N:P molar ratios, with a maximum of 80% knockdown using an 80:1 N:P ratio (FIGS. 14 and 27A). Due to DIVA3 and DIV3H reduced silencing efficiency compared to DIV3W, DIVA3 and DIV3H were not included for protein analysis or CAOV3 qPCR studies. Western blot analysis revealed that expression of CK2α protein was silenced in OVCAR3 cells across all N:P ratios (FIG. 6D) and CAOV3 cells at 40:1 and 80:1 ratios after treatment with DIV3W-siCSNK2A1 complexes in comparison to DIV3W-siNT (FIG. 27B). The significant CSNK2A1 mRNA and CK2α protein knockdown mediated by DIV3W resulted in the selection of this peptide for subsequent anticancer studies.

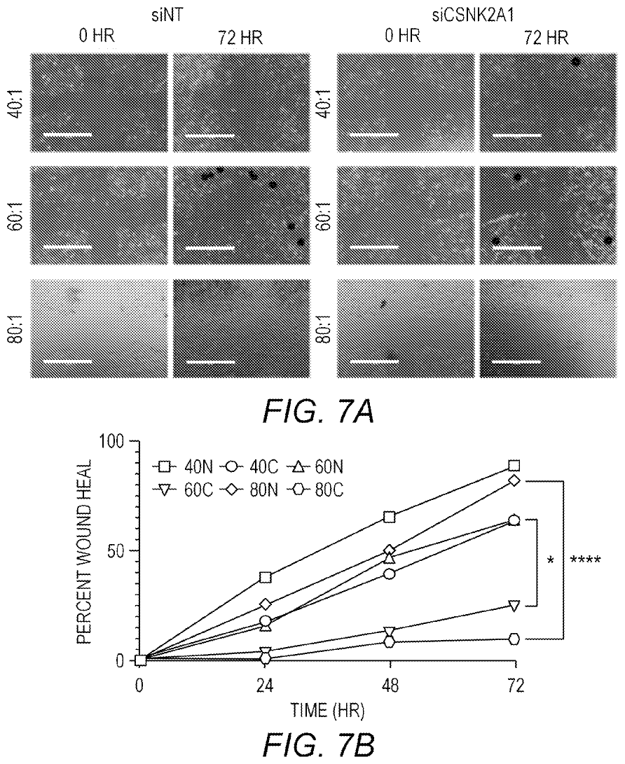

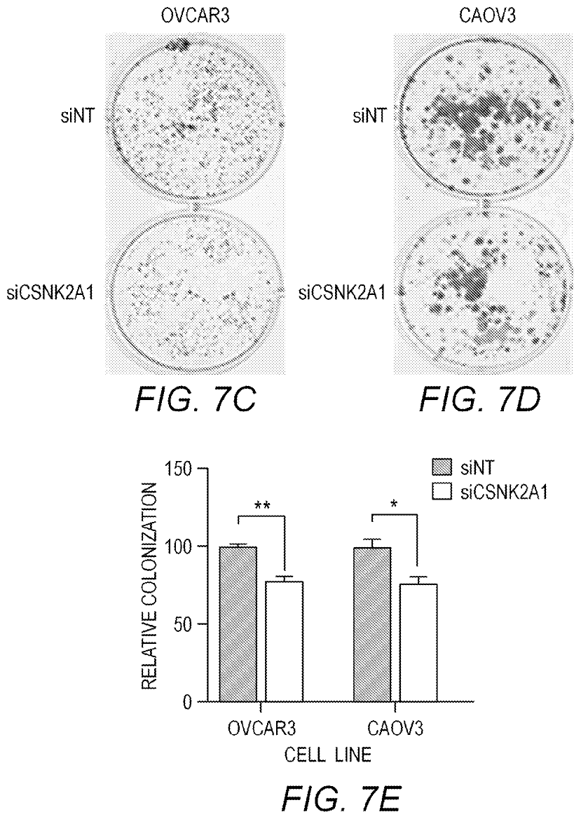

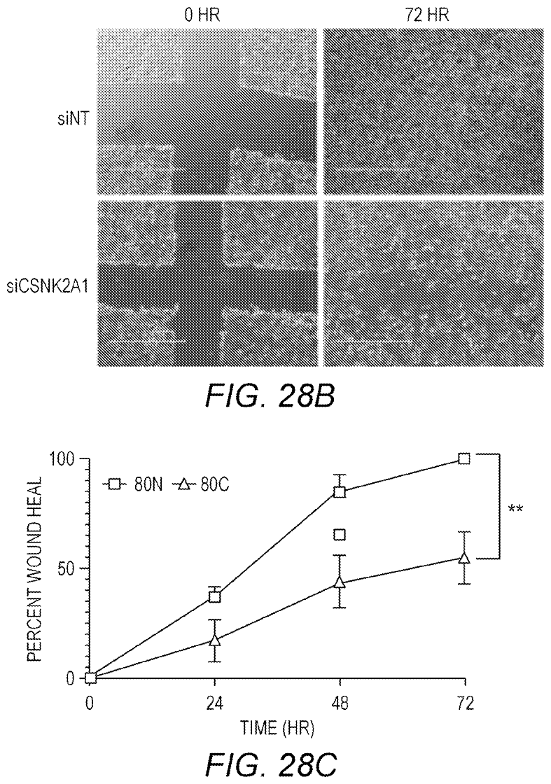

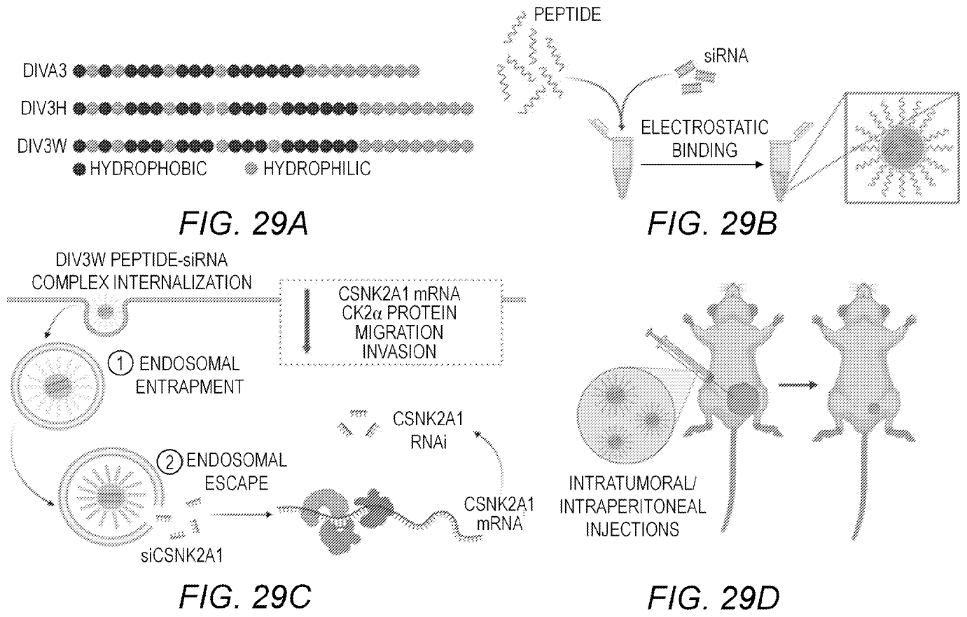

DIV3W peptide-mediated silencing of CSNK2A1 inhibits ovarian cancer cell migration and colonization. Analyzing the anticancer effect of silencing CSNK2A1 mRNA and CK2α protein provided support for utilizing CSNK2A1 as a prospective therapeutic gene target for the treatment of ovarian cancer. Initial evaluation of the effect of CSNK2A1 knockdown on ovarian cancer cell viability using an MTS assay did not produce statistically significant results. To evaluate the effect of silencing CSNK2A1 on cell migration, a scratch wound assay was conducted by first treating OVCAR3 cells with DIV3W-siNT or DIV3W-siCSNK2A1 complexes for 48 hours. Following treatment, a vertical and horizontal scratch wound was introduced on the cell monolayer in each treatment group. Consistent with the uptake and knockdown studies, delivery of DIV3W-siCSNK2A1 complexes at the 40:1 N:P ratio did not exhibit significant inhibition of cell migration compared to treatment with siNT, with cell migration across the scratch area covering 88.8±1.2% of the width in the siNT group and 63.7±15.6% in the siCSNK2A1 group (FIGS. 7A and 7B). Although cells treated with the complexes at the 60:1 N:P ratio did demonstrate some inhibited wound recovery, with 24.7±13.7% of the wound area being repopulated with cells in the siCSNK2A1 group, only the 80:1 ratio demonstrated a statistically significant reduction of migration, with only 9.9±7.2% wound closure in siCSNK2A1 treated cells compared to 82.2±14.7% closure in cells treated with siNT (FIGS. 7A and 7B). CAOV3 cells treated with DIV3W complexes at an 80:1 N:P ratio also displayed a significant reduction after 72 hours, with siCSNK2A1 treatment reducing the scratch wound closure by 45.6%+11.9% in comparison to siNT (FIGS. 28B and 28C).

To determine the effect of CSNK2A1 gene silencing on the long-term proliferative potential of ovarian cancer cells, a clonogenic assay was performed. Treatment of ovarian cancer cells with DIV3W-siNT complexes was compared with DIV3W-siCSNK2A1 complexes at an 80:1 N:P ratio (FIGS. 7C and 7D). OVCAR3 and CAOV3 cells both exhibited a statistically significant decrease in their ability to recolonize, with 78.5±2.5% and 76.4±4.3% relative colonization, respectively, when treated with siCSNK2A1 in comparison to siNT (FIG. 7E). This reduction in cell migration and clonogenicity in both cell lines demonstrated the therapeutic potential of CSNK2A1 as a gene target for ovarian cancer.

### DISCUSSION

Endosomal entrapment of nanocarriers encapsulating siRNA and activation of the lysosomal degradation pathway significantly hinders the translatability of siRNA therapies through acidic degradation. Several nanoparticle systems are able to encapsulate and deliver siRNA to cancer cells but may have reduced biocompatibility and allow insufficient endosomal escape. Cell penetrating peptides cause endosomal escape via the proton sponge effect but have been shown to induce cytotoxic effects on cells because of their strong positive charge. Conversely, lipid and polymer nanoparticles have high biocompatibility but may require the addition of cationic peptides or lipid/polymer formulation changes to increase the charge necessary for endosomal escape capabilities; however, these modifications can hinder uptake and increase cytotoxicity. This work explores novel fusogenic peptides as a viable carrier for RNAi therapeutics that may overcome barriers of cellular uptake, biocompatibility, and endosomal entrapment.

Three fusogenic peptide sequences have been designed with a core amphiphilic sequence repeat to enable endosomal membrane fusion in response to the acidic endosomal environment, each attached to a poly-D-arginine tail to allow efficient complexing of peptides with siRNA cargo through electrostatic interactions. Each DIV3 (X) peptide variant can efficiently complex with siRNAs, form monodisperse nanoparticles, and protect siRNAs from serum and RNase degradation. DIVA3 exhibited siRNA complexation at the lowest N:P molar ratio of 20:1, while DIV3H and DIV3W completely complexed siRNAs at a 40:1 molar ratio. Lower peptide molar ratios necessary for the formation of DIVA3/siRNA complexes may contribute to the smaller hydrodynamic diameter of approximately 85 nm and lower zeta potential, near 20 mV, whereas DIV3H and DIV3W require more peptide to complex siRNAs, contributing to slightly higher diameters around 100 nm and zeta potentials near 30 mV. All DIV3 (X) peptide complexes formed monodisperse nanoparticles with sizes appropriate for intravenous delivery. Analysis of the stability of the DIV3 (X) complexes revealed that each variant was able to protect siRNAs from degradation in the presence of serum and RNase A. Protection from degradation in serum and RNase A provides support for the use of this system in future work to include systemic administration and increased circulation time to allow for the accumulation of nanoparticles in the tumor microenvironment.

After characterizing the peptide/siRNA complexes, the ability of the fusogenic peptides to mediate efficient delivery of siRNAs into ovarian cancer cells in vitro was examined. DIV3W significantly outperformed DIVA3 and DIV3H in enhancing the internalization of siRNAs into OVCAR3 cells. The cellular internalization of siRNAs into ovarian cancer cells increased with increasing N:P ratio for each peptide due to greater hydrophobicity and positive charge. Increases in primary sequence hydrophobicity, and therefore, hydrophobicity of the supramolecular assembled complex, conferred by the tryptophan residue in DIV3W, may contribute to the increased internalization of siRNA by DIV3W compared to the DIVA3 and DIV3H peptides. Peptide sequences including tryptophan were shown to increase cellular membrane affinity and endocytotic activity with increased siRNA internalization mediated by DIV3W. The DIV3W peptide demonstrated siRNA internalization efficiencies of approximately 65% in OVCAR3 cells at each N:P ratio. The differences in endosomal entrapment and later escape, determined by levels of gene knockdown, may be attributed to the increased affinity for pH-dependent conformation change and increased hydrophobicity with the addition of histidine and tryptophan amino acid residues, respectively. Though peptide conformational change to helical secondary structure is required for α-helix formation, inclusion of the tryptophan residue in DIV3W inherently enables higher membrane affinity with increased hydrophobicity before pH changes occur through the insertion of the hydrophobic R groups into the endosomal membrane. It is believed that DIV3W enhances endosomal escape and release of bioactive siRNA into the cytosol. Furthermore, viability studies demonstrated that DIV3 (X) peptides did not cause cytotoxicity alone without siRNA cargo.

The selection of the optimal DIV3 (X) peptide variant was confirmed through qPCR and western blot analysis of siRNA bioactivity. DIV3W proved to significantly enhance bioactivity of siRNAs, as evidenced by increased knockdown of CSNK241 mRNA in ovarian cancer cells in comparison to the other peptide variants. At 60:1 and 80:1 molar ratios, DIV3W-siCSNK2A1 complexes reduced the relative expression of CSNK2A1 mRNA in OVCAR3 cells by approximately 90% and 60%, respectively, compared to treatment with DIV3W complexed with siNT. Although DIVA3 and DIV3H enabled significant knockdown of the target gene at one of three N:P ratios, they did not cause comparable knockdown to DIV3W peptide-mediated delivery. Protein knockdown downstream of mRNA degradation was confirmed following DIV3W-siCSNK2A1 delivery in ovarian cancer cells. DIV3W-siCSNK2A1 complexes exhibited significant knockdown of CK2α protein expression at all N:P ratios. The bioactivity mediated by the DIV3 (X) peptides also reflected the stability of the complexes in serum, since all gene silencing experiments were conducted in the presence of serum to replicate physiologically relevant conditions. The superior siRNA bioactivity achieved with DIV3W compared to the other peptide variants aligns with previous assays, as DIV3W also demonstrated the highest siRNA protection and endosomal escape. These results demonstrate the potential of DIV3W as a delivery system for RNAi-based therapies and justify the selection of DIV3W as the fusogenic peptide for analysis of ovarian cancer cellular response to silencing the target gene.

CSNK2A1 has a vast array of roles in the activation of cellular pathways and processes. Many of these responses may be cancer type-dependent; studies have shown that CSNK2A1 activates phosphorylation of SIRT6 in breast cancer, the PI3K-Akt-mTOR pathway in gastric cancer, and NF-κB in glioblastoma, all contributing to poor patient prognosis. In order to examine the effect of silencing CSNK2A1 expression on oncogenic activity in ovarian cancer cells, viability, scratch migration, and colonization assays were completed after treatment with DIV3W-siCSNK2A1 complexes. MTS viability experiments did not show any significant loss of cell viability after a 48-hour treatment with DIV3W-siCSNK2A1 complexes compared to cells treated with DIV3W-siNT. Other studies have also demonstrated that knocking down CSNK2A1 does not result in a loss in cell viability without including supplemental drug treatments due to the many roles of CSNK2A1 in cell communication pathways in different cell types. Though silencing CSNK2A1 did not affect cell viability, the results did demonstrate that silencing CSNK2A1 significantly inhibited the migration of ovarian cancer cells. Reduction in cell migration due to silencing CSNK2A1 is consistent with previous reports that show upregulated CSNK2A1 expression contributes to increased cancer aggressiveness and decreased survivability rates in several cancer types. Specifically, it has been demonstrated that CSNK2A1 expression mediates the PI3K-Akt-mTOR pathway, attributing to increased oncogenesis, migration, and invasion. Knockdown of CSNK2A1 via lentiviral transfection in gastric cancer cells inhibited migration across a scratch wound in comparison to cells with high CSNK2A1 expression. It was also observed that CSNK2A1 knockdown in gastric cancer cells reduced their invasion, which may correlate to decreased cancer cell regrowth after treatment, reducing distant metastatic spread. Additionally, studies have shown that overexpression of CSNK2A1 and its protein product, CK2a, enhance the clonogenic survivability of melanoma cells. The results herein are consistent with this observation; silencing of CSNK2A1 mediated by the DIV3W peptide significantly reduced the clonogenicity of OVCAR3 and CAOV3 ovarian cancer cells. This consistency between cancer models also suggests that CSNK2A1's roles in tumorigenesis can possibly be narrowed to cell communication, enhancing migration and regrowth.

Overall, these results demonstrate the ability of a series of fusogenic peptides, comprised of amphiphilic core repeats and a cationic poly-(D)-arginine tail, to efficiently complex with and deliver siRNA into ovarian cancer cells. The fusogenic peptides are able to self-assemble with siRNAs to form monodisperse nanoparticles and protect siRNAs from serum and RNase-mediated degradation. The DIV3W peptide proved optimal for enhancing intracellular delivery, endosomal escape, and silencing of CSNK2A1 mRNA and CK2α protein expression. Consequently, delivery of bioactive siCSNK2A1 resulted in significant inhibition of cancer cell migration and clonogenicity. These results demonstrate the potential of the DIV3W peptide for the delivery of RNAi therapeutics and the potential of CSNK2A1 as a therapeutic gene target to reduce ovarian cancer invasiveness.

### Example 2

Though Example 1 demonstrates the potential of DIV3W for enabling endosomal escape of siRNA cargo and highly efficient knockdown of target genes, delivery of cargo is nonspecific. Thus, to increase cell and tissue specificity, a targeting peptide was included within the fusogenic delivery system. The tandem peptide will not only enhance cellular uptake, but will also implement the fusogenicity of the DIV3W peptide to cause endosomal membrane destruction and release of bioactive siRNAs.

The current work highlights the propensity of a targeting peptide for the luteinizing hormone releasing hormone (LHRH) receptor, also known as gonadotropin releasing hormone (GNRH), to enhance the ability of DIV3W to mediate targeted delivery of bioactive siRNAs into ovarian cancer cells. LHRH receptor is a commonly overexpressed cell receptor in several cancers, including epithelial ovarian cancer. By introducing the LHRH peptide sequence to create a tandem peptide with DIV3W, it is hypothesized that uptake will be enhanced in cells overexpressing LHRH receptor, while also enabling endosomal escape of bioactive siRNA. The LHRH peptide, complexed to DIV3W via a glycine-linker, enabled targeted delivery of siRNAs into ovarian cancer cells through active targeting, enhancing uptake of siRNAs via receptor-mediated endocytosis after specific binding interactions with the LHRH receptor. In addition, the tandem LHRH-DIV3W peptide delivered bioactive siRNAs, causing significant CSNK2A1 gene knockdown and resulting in downstream reduction of ovarian cancer cell migration and recolonization. The ability of the tandem peptide to cause receptor-specific binding and endocytosis of siRNA cargo, in addition to resultant gene knockdown and reduction in cell recolonization, shows the potential for this system to be a potent delivery vehicle in in vivo or clinical applications.

### Materials and Methods

Cell Culture. Ovarian epithelial cancer cell lines, OVCAR3, obtained through the American Type Culture Collection (ATCC, Manassas, VA) and CAOV3 (Hollings Cancer Center, Charleston, SC) were cultured in McCoy's 5A culture medium and Dulbecco's Modified Eagles Medium (DMEM) (ATCC), respectively. These cultures were supplemented with 10% fetal bovine serum (FBS) (Corning, Corning, NY) and 1% penicillin/streptomycin (P/S) antibiotic solution (Corning). All cultures were incubated at 37° C. in 5% CO₂. Cell lysate of the healthy ovarian surface epithelial cell line (HOSEpiC, ScienCell, Carlsbad, CA) was stored at −80° C.

Peptide Synthesis. Peptides, DIV3W (WEADIVADIVWDIVADIVAGGG-(d) RRRRRRRRR) (SEQ ID NO:3), LHRH (pGHWSY-(d) L-LRP) (SEQ ID NO:4), and LHRH-DIV3W (pGHWSY-(d) LLRPGGGGGWEADIVADIVWDIVADIVAGGG-(d) RRRRRRRRR) (SEQ ID NO:5), were synthesized, purified (>95% purity), and analyzed via high performance liquid chromatography (HPLC) at Genscript USA Inc. (Genscript, Piscataway, NJ). All formulations contained C-terminal sequence of nine D-arginine (dR) residues with no terminal modifications. Peptide formulations were dissolved, diluted, and stored as previously described.

siRNAs. The siRNA targeting CSNK2A1 (siCSNK2A1) was synthesized by Thermo Fisher Scientific (#s3638, Thermo Fisher Scientific, Waltham, MA). Negative control siGENOME non-targeting siRNA #5 (siNT) and Cy5.5 fluorescently labeled siNT (siNT-Cy5.5.5) were purchased from GE Healthcare-Horizon Discovery, Dharmacon (GE Healthcare Dharmacon, Lafayette, CO).

Characterization of Nanoparticle Size and Stability. To determine the minimum molar ratio necessary to completely complex free siRNAs, increasing N:P molar ratios of LHRH, DIV3W, and LHRH-DIV3W peptide were complexed with siRNAs and electrophoresed using a electrophoretic mobility shift assay. Molar ratios, shown as N:P, are representations of the molar amine peptide content in relation to 1 mole of phosphate group siRNA content. To determine the ability of the peptides to protect siRNAs from serum- or RNase-mediated degradation, LHRH and LHRH-DIV3W complexes were formed in RNase-free water with siNT at increasing N:P molar ratios and incubated at 37° C., 5% CO₂, 50% v/v FBS, 5 μg/mL RNase A, or 50% v/v RNase free water for 1 hour. Peptide/siRNA complexes were dissociated using 6% sodium dodecyl sulfate (SDS) prior to electrophoresis in a 2% agarose gel run at 100 V. Gels were stained with ethidium bromide, washed with Milli-Q water, and imaged with a BioRad ChemiDoc XRS imager (BioRad, Hercules, CA).

To determine nanoparticle size and zeta potential, peptides resuspended in RNase-free water (10 μM) were thawed and kept on ice prior to complexation with 10 μM of siNT in RNase-free water and incubated for 20 minutes at room temperature to form DIV3W-siNT and LHRH-DIV3W-siNT complexes. Peptide complexes were prepared at 2 mg/mL in RNase-free water. Hydrodynamic size and surface charge analysis was conducted using a Malvern Nano ZS Zetasizer (Malvern Panalytical, Malvern, UK). All data was analyzed using Malvern's Zetasizer software.

Peptide Biocompatibility. OVCAR3 or CAOV3 cells were seeded at 10,000 cells per well with complete culture media in a transparent 96-well plate and incubated overnight to allow the cells to attach. LHRH and LHRH-DIV3W peptides were delivered to cells without siRNA cargo. After 24 hours of incubation the media was exchanged and after another 24-hour incubation at 37° C. in 5% CO₂, viability was measured using an MTS Assay performed with the CellTiter 96 AQueous One Solution Assay (Promega, Madison, WI). Assay reagent was added according to the manufacturer's protocol and cells were incubated for 4 hours. Absorbance was read using a BioTek Synergy LX (BioTek, Winooksi, VT) plate reader at 490 nm.

Immunofluorescence of LHRH Receptor Expression. Visualization of LHRH receptor expression in OVCAR3 and CAOV3 cells was completed by seeding cells at 50,000 cells per well in a 24-well plate. Cells were fixed with 4% paraformaldehyde for 15 minutes at room temperature and washed three times with 1× PBS. After washing, cells were blocked with 1% bovine serum albumin (BSA) solution for 30 minutes at 4° C. Three additional 1×PBS washes were completed and cells were then incubated with GnRHR primary antibody at 1:100 dilution (Invitrogen, Carlsbad, CA) overnight at 4° C. in the dark. After primary antibody incubation, cells were once again washed with 1×PBS and subsequently incubated with Secondary SuperSignal Alexa Fluor 488 antibodies (Invitrogen) according to the manufacturer's protocol. Nuclear stain was also performed with SuperSignal NucBlue according to Invitrogen protocol and cells were imaged at 20× using an Evos FL microscope (Thermo Fisher).

Western blotting of LHRH receptor expression in OVCAR3 and CAOV3 cells was completed by seeding cells at 100,000 cells per well in a 6-well plate. Cells were washed and lysed with cold radioimmunoprecipitation assay (RIPA) buffer supplemented with protease inhibitor cocktail (Thermo Fisher). Protein concentrations were determined via bicinchoninic assay according to the manufacturer's protocol and absorbance was read at 562 nm. OVCAR3, CAOV3, and HOSEpiC (ScienCell) protein isolate at equivalent amounts was separated by SDS-PAGE on a BioRad Stain-Free 10% gel according to BioRad protocols and then transferred to a Stain-Free PVDF membrane. PVDF was cut at appropriate molecular weight intervals and blocked for 1 hour with 5% non-fat dried milk solution in 1× Tri-HCl-buffered-saline-0.1% Tween 20 (TBST) at room temperature. Mouse anti-GnRHR antibody (1:1000, Invitrogen) and mouse anti-β-actin (1:10,000, Sigma-Aldrich, St. Louis, MO) was incubated with membranes at 4° C. overnight. Membranes were washed three times with 1× TBST and incubated with goat anti-mouse secondary antibodies (1:1000 or 1:10,000, Thermo Fisher) for 1 hour at room temperature. Five additional washes with 1× TBST were performed, and protein bands were detected with SuperSignal West Pico Plus Chemiluminescent Substrate (Thermo Fisher) and imaged using a BioRad ChemiDoc Imaging System.

Cellular Uptake and Targeting Specificity. Observation of cell uptake was performed by seeding OVCAR3 and CAOV3 cells at a density of 50,000 cells per well in a 24-well plate. LHRH and LHRH-DIV3W peptides were complexed with siNT-Cy5.5.5 as previously described and delivered to cells at 20:1 to 80:1 N:P ratios. Each cell line was incubated with tandem peptide complexes for both 4- and 24-hour treatment periods. Following incubation, cell media was removed, and cells were washed with 1× PBS prior to the addition of 500 μL of 1×PBS and SuperSignal NucBlue Hoescht 33342 nuclear stain (Thermo Fisher, Waltham, MA). Cellular imaging was completed using an EVOS FL Microscope (Thermo Fisher) at 10×, 20×, and 40× magnification. Flow cytometry was completed after cell imaging and cells were trypsinized, resuspended in 1×PBS, and analyzed with an Attune NXT Acoustic Flow Cytometer (Invitrogen) with the red laser line and RL2 (wavelength) channel.

To analyze the specificity of the LHRH-DIV3W tandem peptide targeting LHRH receptor, a competitive binding assay was performed by incubating OVCAR3 and CAOV3 cells with GnRHR primary antibody (1:100) in 1× PBS overnight at 4° C. after being blocked with a 1% bovine serum albumin solution for 30 minutes at room temperature. LHRH-DIV3W-siNT-Cy5.5 complexes were delivered after the overnight primary antibody incubation and incubated again for 4 hours. Secondary SuperSignal Alexa Fluor 488 antibodies were delivered dropwise to cells following Invitrogen SuperSignal protocols and left to incubate at room temperature for 30 minutes and subsequently stained with SuperSignal NucBlue Hoescht 33342 for an additional 30 minutes at room temperature. Cells were imaged at 20× and 40× using an Evos FL microscope (Thermo Fisher).

Gene and Protein Knockdown. Bioactivity of siRNAs was determined via qPCR and western blot analysis. OVCAR3 or CAOV3 cells were seeded at 50,000 cells per well as previously described and treated with siRNA targeting CSNK2A1 and siNT at N:P ratios 30:1, 40:1, 50:1, 60:1, and 70:1. LHRH-DIV3W peptide complexed with either siNT or siCSNK2A1 was incubated for a total of 48 hours in RPMI 1640/DMEM-10% FBS, with one media exchange occurring after the 24-hour timepoint. Treated cells were washed with 1×PBS at 24 hours and fresh media was added. RNA isolation was completed using a Qiagen Mini-RNeasy Kit (Qiagen, Hilden, Germany) according to the manufacturer's protocol. RNA isolation quality and quantity was measured through the Gen5 protocol using a Take3 plate (BioTek). RNA was reverse transcribed using the QuantiTect Reverse Transcription kit (Qiagen) according to the manufacturer's protocol. Quantitative real-time PCR was completed with a QuantStudio 3 qPCR system (Applied Biosystems, Waltham, MA) using Applied Biosystems TaqMan FAST Master Mix (Thermo Fisher) and CSNK2A1 (ID #Hs00751002_s1) and 18S (ID #Hs99999901_s1) predesigned TaqMan probes according to the manufacturer's protocol.

Clonogenic Recolonization. Clonogenicity, or the ability for cells to recolonize a large area, was analyzed to determine the ability of cells to recover and regrow into viable colonies after treatment with LHRH-DIV3W-siNT or siCSNK2A1. Recolonization of cells harvested after treatment with LHRH-DIV3W-siNT or siCSNK2A1 was analyzed via a clonogenic assay and quantified through absorbance spectroscopy using a BioTek Synergy LX plate reader (BioTek) at 490 nm. OVCAR3 cells were seeded at 50,000 cells per well in a 24-well plate and treated with N:P ratios 30:1, 40:1, 50:1, 60:1, and 70:1 of LHRH-DWIV3W-siNT or siCSNK2A1 complexes. After a 48-hour period, cells were harvested, centrifuged into a pellet, and reseeded at 2,500 cells per well in a 6 well plate, yielding 1.25 cells/μL. All treatment groups were allowed to colonize for a 14-day period with 1×PBS washes and media changes at 2-day intervals. Following the growth period, cells were stained using a 0.1% crystal violet solution for 30 minutes, washed with ddH₂O, and left to rest in ddH₂O overnight at 4° C. The following morning, ddH₂O was removed and cells were dried for 15 minutes before being UV imaged with a ChemiDoc Imaging System using the white light filter. Quantification was completed by dissolving the stained cells in 33% acetic acid and transferring 100 μL of each sample to a 96-well plate in triplicate. Well absorbance was measured at 490 nm.

### Results

The Targeting and Tandem Peptides form Monodisperse Nanoparticles and are Biocompatible. The LHRH receptor targeting peptide, a sequence implemented in several cancer targeting research efforts, was conjugated to the DIV3W peptide via a flexible penta-glycine linker to form a tandem peptide as shown in FIG. 8A. Results of the gel shift assay showed that the tandem LHRH-DIV3W peptide electrostatically bound with siRNAs at a minimum molar ratio of 20:1 (FIG. 8B); thus, N:P ratios of 20:1 to 80:1 were used for subsequent experiments. DLS results demonstrated that LHRH-DIV3W peptide complexes formed the largest nanoparticles compared to DIV3W and LHRH peptide complexes at 154.2±15.9 nm with a polydispersity index of 0.18 (FIGS. 8C and 8E), demonstrating formation of monodisperse nanoparticles. Additionally, the tandem peptide complexes exhibited the highest positive charge when compared to LHRH and DIV3W complexes, at 33.37±2.00 mV. After confirmation of nanoparticle formation, LHRH-DIV3W was evaluated for its ability to protect siRNA in physiological conditions. Simulation of physiological conditions was performed by incubating the LHRH-DIV3W-siNT complexes in 50% fetal bovine serum (FBS) or RNase A and detecting in-tact siRNAs using electrophoretic gel shift (FIG. 8D). Each N:P ratio incubated with 50% FBS displayed high protection of siRNAs evidenced by bright white banding. However, RNase A degradation protections required increased N:P ratios, with degradation of siRNAs appearing at 20:1 and 30:1 in comparison to higher 40:1 and 50:1 N:P ratios.

Ensuring the biocompatibility of these nanoparticles was pivotal in understanding that any anticancer effects observed downstream were resulting from RNAi mechanisms and not peptide cytotoxicity. It was determined that LHRH-DIV3W did not cause cytotoxic effects in OVCAR3 and CAOV3 cells when delivered without siRNA cargo, but did cause a slight statistically significant increase in viability in OVCAR3 cells at the 20:1 ratio with viability measured at 109.66%+0.8% (FIG. 8F). At N:P ratios greater than 20:1, viability values returned to insignificant levels in comparison to untreated controls, suggesting mitogenesis is rate limited by LHRH receptor binding capacity.

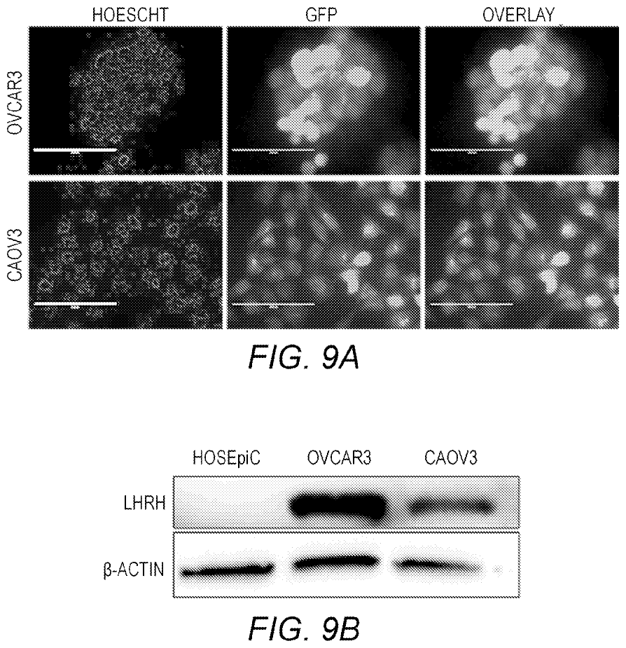

LHRH Receptor is Overexpressed in OVCAR3 and CAOV3 Cells. LHRH receptor expression in ovarian cancer confirmed that both OVCAR3 and CAOV3 cells exhibited expression of LHRH receptor when analyzed with immunofluorescent microscopy (FIG. 9A). For comparison to a healthy epithelial cell line, HOSEpiC cell lysate was blotted for LHRH receptor, in addition to OVCAR3 and CAOV3 lysate. HOSEpiC cells did not display a protein band for LHRH receptor, while OVCAR3 and CAOV3 both expressed LHRH (FIG. 9B) protein in direct correlation with the immunofluorescence imaging. OVCAR3 had the highest LHRH receptor expression between the three cell lines.

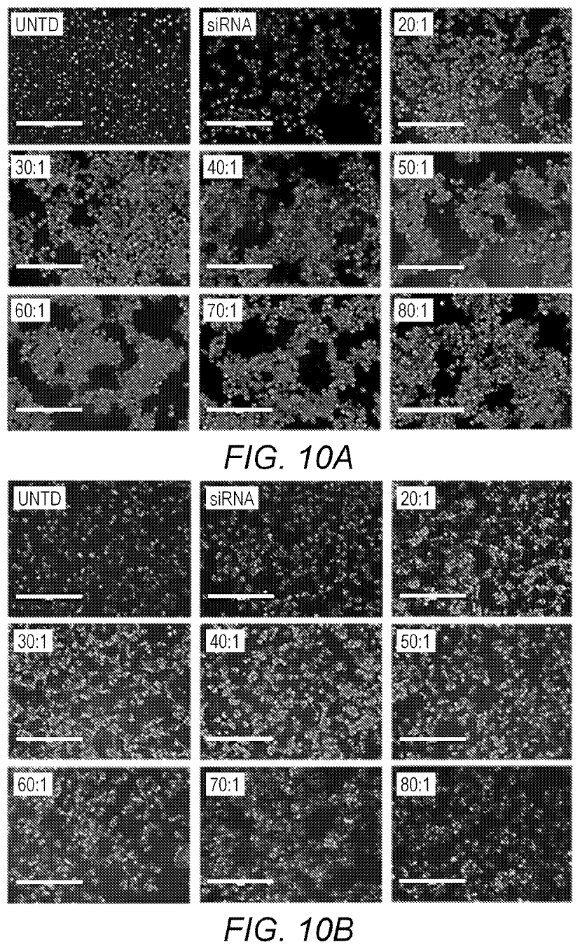

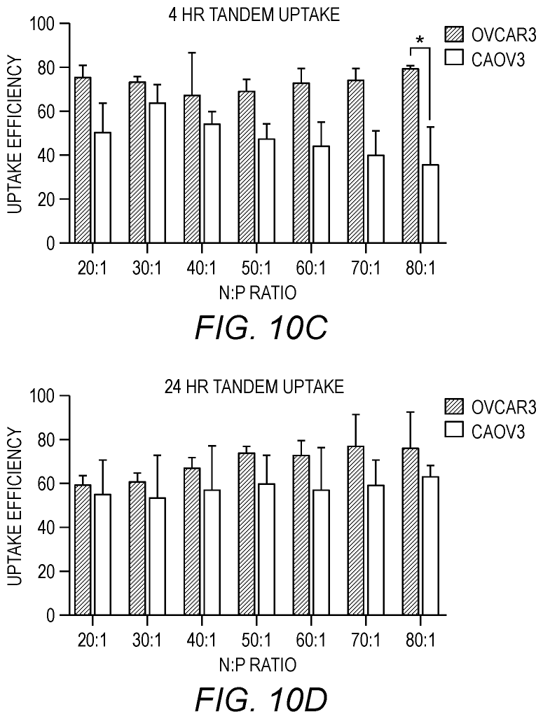

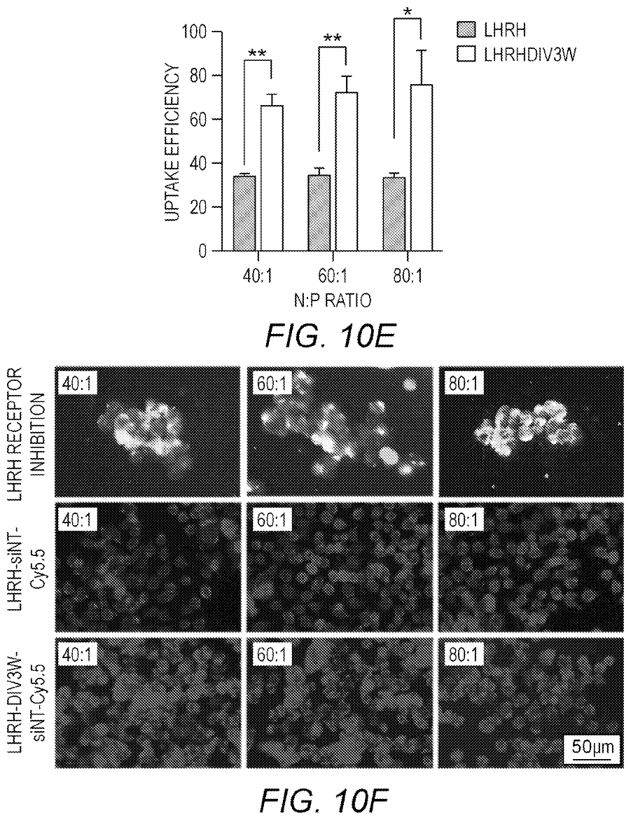

The LHRH-DIV3W Tandem Peptide Specifically Targets the LHRH Receptor on Ovarian Cancer Cells. Cellular uptake of siRNAs via active targeting mechanisms is the major goal of including the LHRH peptide with the DIV3W fusogenic sequence, forming the LHRH-DIV3W peptide. Active targeting and uptake were examined using immunofluorescent imaging and flow cytometry. The LHRH-DIV3W peptide complexed with siNT-Cy5.5.5 was delivered to OVCAR3 CAOV3 cells and exhibited increasing cell internalization with increasing N:P ratios for all treatment groups except 50:1 to 80:1 in CAOV3 after 4 hours (FIGS. 10A and 10B). Quantitation using flow cytometry showed OVCAR3 uptake efficiencies reaching 79.58%+0.61% at the 80:1 ratio, which was significantly greater than CAOV3 cell uptake efficiency at the same ratio, reaching 36.19%+9.67% (FIG. 10C). However, after 24-hour incubation with tandem-siNT-Cy5.5 complexes, this discrepancy was no longer observed and CAOV3 cells also had consistent uptake efficiency levels, reaching 63.47%+4.61% at the 80:1 ratio (FIG. 10D). Compared against the LHRH peptide alone, LHRH-DIV3W tandem significantly improved cell uptake at the ratios 40:1, 60:1, and 80:1 with uptake efficiencies of 66.81%±4.3%, 72.59%±6.87%, and 76.26%±14.9%, respectively (FIG. 10E). LHRH-mediated uptake efficiencies at these same ratios were 34.3%±0.56%, 34.83%±2.75%, and 33.87%±1.63%, respectively.

To confirm that the LHRH-DIV3W peptide binds specifically to the LHRH receptor, comparative imaging was completed with OVCAR3 and CAOV3 cells first incubated with LHRH receptor antibody. Preincubation with LHRH receptor antibody would ensure LHRH receptor-antibody binding, blocking the binding of the LHRH-DIV3W peptide. FIG. 10F and FIG. 14 show that when both cell lines were preincubated with LHRH receptor antibody, siRNA internalization was drastically decreased.

LHRH-DIV3W Mediates Delivery of Bioactive siRNAs at Higher N:P Molar Ratios. The DIV3W fusogenic peptide can deliver bioactive siRNAs and mediate silencing of CSNK2A1 in OVCAR3 and CAOV cells. Thus, whether the tandem peptide, containing LHRH conjugated via a penta-glycine linker to DIV3W, retained the ability to delivery bioactive siRNAs into ovarian cancer cells was examined. qPCR results revealed silencing of CSNK2A1 mRNA expression mediated by LHRH-DIV3W-siCSNK2A1 at 50:1, 60:1, and 70:1 N:P ratios, resulted in statistically significant knockdown of 48.2% at a 70:1 ratio in OVCAR3 cells (FIG. 11A). 50:1 and 60:1 ratios of LHRH-DIV3W-siCSNK2A1 yielded a knockdown of 40.3% and 27.8%, respectively. Western blot analysis of CK2α protein also revealed decreased protein expression in 50:1 N:P ratio (FIG. 11B).

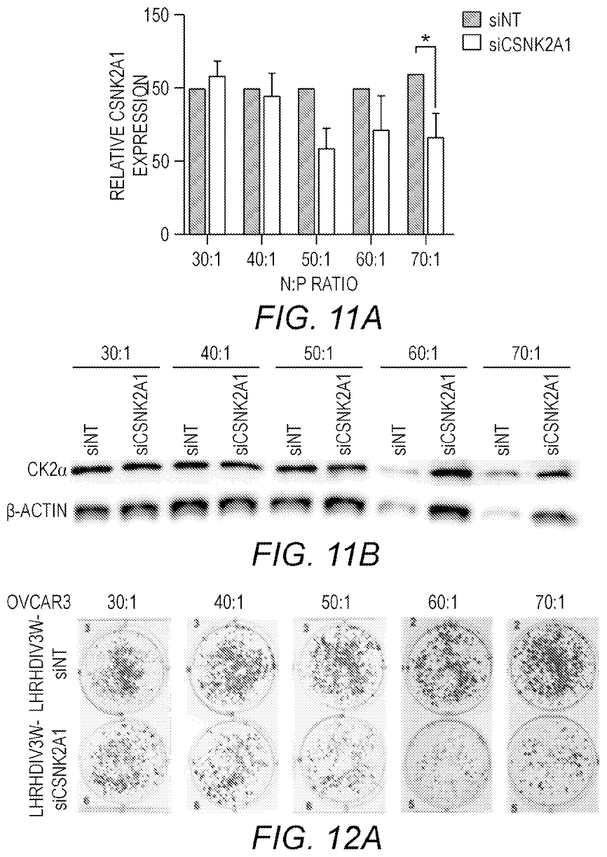

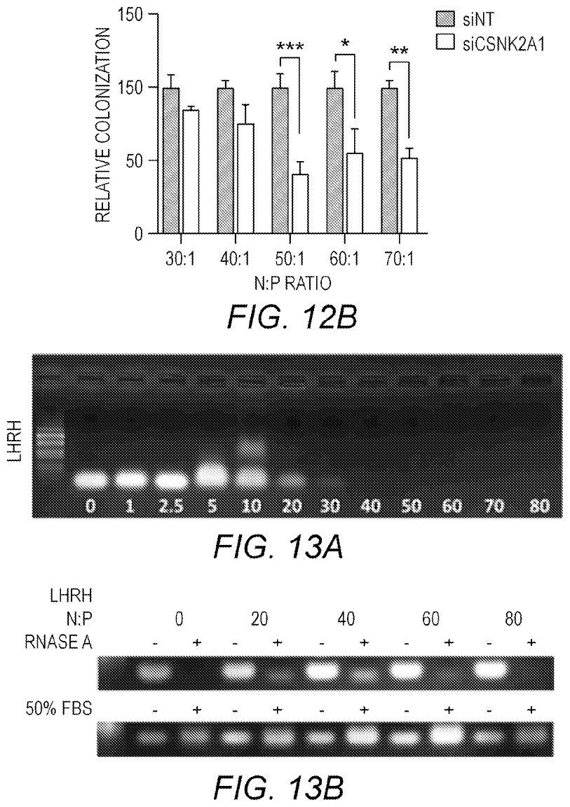

Recolonization Responds to Low Knockdown Levels Without Loss of Viability or Migration. Characterization of cell response to knocking down the CSNK2A1 gene is an important aspect of determining the clinical applications for this gene target in ovarian cancer. Silencing of CSNK2A1 results in reduction of cell migration and recolonization in ovarian cancer cells. Clonogenic recolonization of OVCAR3 cells treated with LHRH-DIV3W-siSCNK2A1 displayed a significant reduction in colonization compared to treatment with LHRH-DIV3W-siNT at 50:1, 60:1, and 70:1 ratios with only 41.9%±7.8%, 56.8%±15.3%, and 52.5%±6.7% colonization shown for each ratio, respectively (FIGS. 12A and 12B).

### DISCUSSION

The design of the LHRH-DIV3W peptide takes advantage of the functional properties of the LHRH and DIV3W peptides to create a tandem peptide with both targeting and fusogenic properties to enhance delivery of bioactive siRNAs. LHRH receptor has been successfully targeted in ovarian cancer drug delivery systems and enhances cell internalization through its receptor-mediated endocytotic behavior. Many drug and gene delivery systems have utilized targeting peptides as complementary systems to enhance active targeting in the tumor microenvironment or to evaluate systemic routes of therapeutic delivery. Identified and synthesized primarily via phage display techniques, targeting peptides have the capacity to continue to expand in their uses as more overexpressed cellular receptors are identified on cancer cells. Though targeting is advantageous for increasing therapeutic delivery, another significant barrier to RNAi therapeutic efficacy is endosomal escape after targeted uptake has taken place. Thus, additional components must be added to create a robust delivery system capable of not only targeted delivery, but delivery of bioactive therapeutics.

This work introduces the use of an LHRH receptor targeting peptide as a complement to the DIV3W fusogenic peptide that shows the ability to deliver bioactive siRNAs in vitro and in vivo. The LHRH and DIV3W peptides are joined through the inclusion of a flexible penta-glycine linker at the junction between the sequences and another glycine linker is included at the junction between DIV3W and the arginine tail, a cationic sequence necessary for electrostatic binding of anionic siRNAs. Characterization of the tandem system revealed the ability of the LHRH-DIV3W peptide to completely bind siRNAs starting at an N:P ratio of 20:1 and form monodisperse nanoparticles sized 154.2±15.9 nm. This allows for passive targeting through the enhanced permeability and retention effect in addition to active targeting via the LHRH receptor. Compared to DIV3W and LHRH peptides alone, the tandem system formed the largest, most monodisperse nanoparticles and contained the highest surface charge, which is attributed to the longer amino acid sequence in the tandem system as well as the combinatorial effect of cationic residues in both the LHRH and DIV3W regions. While complexation efficiency is pivotal, it is also important to show that the tandem system can effectively protect the siRNA cargo from degradation when exposed to RNases and/or serum. The LHRH-DIV3W peptide was able to fully protect siRNA cargo from degradation in a 50% serum environment, shown as white banding indicating intact siRNA. Higher N:P ratios were required for full protection of siRNAs when exposed to RNase A. Similar to results seen using the DIV3W fusogenic peptide, enhanced ability for the peptide systems to protect siRNA cargo in these simulated physiological environments provides support for their systemic use in vivo by increasing circulation time and allowing the targeted delivery system to navigate to the target tissue site.

Before using the tandem peptide to deliver therapeutics, it is important to ensure that the delivery vehicle is not causing cytotoxic effects on its own. The results demonstrated that the tandem peptide can be delivered without causing reduction in cell viability in OVCAR3 and CAOV3 cells. However, due to the binding of the LHRH-receptor, it was observed that an increase in cell viability occurs at a 20:1 N:P ratio. LHRH is known to be an activator of cell communication with possible enhanced cell proliferation. However, it was observed that changes in viability were insignificant compared to untreated cells at N:P ratios greater than 20:1.

The goal of incorporating the LHRH peptide with the DIV3W fusogenic peptide system was to enhance targeted delivery of siRNAs. The data allowed visualization and quantification of the efficiency of siRNA delivery via the tandem complex through fluorescence microscopy and flow cytometric analyses. The LHRH-DIV3W peptide enabled enhanced siRNA uptake in both OVCAR3 and CAOV3 cells after 4 hours compared to the LHRH peptide alone. Consistent with delivery by the DIV3W peptide, siRNA uptake increased linearly with increasing N:P ratios for OVCAR3 cells and a statistically significant difference in uptake efficiency between OVCAR3 and CAOV3 was observed at an 80:1 ratio. As expected, the flow cytometry results and uptake imaging of the tandem peptide complexes directly correlated with the level of LHRH receptor expression between the two cell lines used. CAOV3 cells exhibited a lower LHRH receptor expression in comparison to OVCAR3 cells and is reflected in the reduced ability of CAOV3 cells to bind with the tandem complex at higher N:P ratios over the short time period of 4 hours. This can be explained by the crowding of nanoparticles at few LHRH-receptor sites with each nanoparticle competing for fewer available receptors in comparison to the availability of binding sights in OVCAR3 cells, shown in the immunofluorescence and western blot characterization of LHRH receptor expression levels between OVCAR3 and CAOV3 cell lines. To confirm this effect, uptake of the tandem peptide complexes was also analyzed after 24 hours. The increased time-period allowed CAOV3 cells to recycle the LHRH receptor and caused increased levels of siRNA internalization, from 36.19%±16.76% after 4 hours to 63.4%±4.6% after 24 hours at an 80:1 N:P ratio. In contrast, OVCAR3 cells, which had higher LHRH receptor expressed, exhibited high uptake efficiencies at both time points, where 80:1 N:P ratio yielded 79.58%±1.05% uptake at 4 hours and 76.26%±15.56% uptake at 24 hours. Compared to DIV3W uptake, LHRH-DIV3W complexes mediated uptake efficiencies 12% higher in OVCAR3 cells and 11% higher in CAOV3 cells.

Though high levels of siRNA uptake was evident through fluorescence imaging and flow cytometry, it is also crucial to ensure that the LHRH-DIV3W peptide specifically targets the LHRH receptor. Evaluation of the targeting specificity was performed through inhibiting the LHRH receptor using an LHRH receptor antibody. LHRH-bound cells exhibited minimal siRNA internalization in comparison to cells treated with LHRH-DIV3W complexes without receptor inhibition in both OVCAR3 and CAOV3 cells. Through these observations, it was confirmed that the tandem peptide complex effectively targeted the LHRH receptor, causing increased receptor-mediated endocytosis and internalization of siRNAs.

After confirming active targeting of the LHRH receptor, the ability of the tandem peptide to mediate delivery of bioactive siRNAs was examined through western blot and qPCR analysis. When delivered at high N:P ratios, the LHRH-DIV3W peptide was able to achieve statistically significant knockdown of the CSNK2A1 gene, with the 70:1 ratio reducing mRNA expression to 51.88%±9.87% of the negative control, which was treated with LHRH-DIV3W-siNT. Ratios of 60:1 and 50:1 also caused gene knockdown to 72.17%±23.19% and 59.75%±12.61%, respectively. Additionally, western blot analysis displayed slight reduction in protein band intensity at the 50:1 ratio. Due to the reduction in protein with the negative control at the 60:1 and 70:1 ratios, conclusions could not be drawn on the protein silencing at each of those ratios using siCSNK2A1. Compared to the fusogenic DIV3W peptide, the LHRH-DIV3W tandem delivery did not cause an equivalent amount of CSNK2A1 mRNA reduction. The DIV3W peptide is able to efficiently escape the endosome and cause mRNA reduction of up to 94%.

A full understanding of CSNK2A1 pathways is yet to be determined, but it is known that that gene plays a role in increasing cancer aggressiveness and oncogenesis through PI3K-Akt-mTOR pathway activation. It has been demonstrated herein that silencing CSNK2A1 reduces cell migration and recolonization in ovarian cancer cells. Although knockdown of CSNK2A1 mediated by LHRH-DIV3W-siCSNK2A1 was not as efficient as that observed with DIV3W-siCSNK2A1, the tandem peptide complexes did still cause reduction in cell migration and recolonization ability. When seeded at very low densities, the ability for cells to recolonize simulates tumor resection where clear margins may be difficult to obtain. Residual cancerous cells may remain and without effective treatment recolonize and grow, resulting in disease recurrence, a common complication in late-stage ovarian cancer patients. Treatment of these cells with LHRH-DIV3W-siCSNK2A1 significantly reduced the ability of OVCAR3 cells to recolonize at low densities in comparison to cells treated with LHRH-DIV3W-siNT. Additionally, silencing of CSNK2A1 caused a significant reduction in ovarian cancer cells migration across a scratch wound, lending support to the role of CSNK2A1 in cell-cell communication and implying a reduced risk of metastasis. These results are consistent with the delivery of DIV3W-siCSNK2A1 and provide support for the clinical relevance of CSNK2A1 as a therapeutic gene target.

This work sought to improve upon the delivery and bioactivity of siRNA therapeutics for ovarian cancer by incorporating a peptide targeting the LHRH receptor with the fusogenic DIV3W peptide. The results confirmed the ability of this tandem peptide, LHRH-DIV3W, to efficiently complex with and protect siRNA cargo in physiological conditions as well as form monodisperse nanoparticles. Incorporation of the LHRH targeting peptide increases siRNA uptake efficiency through specific binding of the LHRH receptor, but exhibited decreased endosomal escape compared to the DIV3W peptide, also resulting in reduced mRNA and protein knockdown when compared to DIV3W-mediated delivery of siCSNK2A1. However, the knockdown achieved was still sufficient in causing reduction in cell migration and recolonization and continue to support the potential of CSNK2A1 as a therapeutic target for ovarian cancer treatment. Additionally, the achievement of active targeting using the LHRH peptide sequence to form a tandem peptide can assist in increasing endocytotic uptake of siRNA cargo. With enhanced targeting and manipulation of the linker sequence to allow for high fusogenicity via the DIV3W region, there is potential for great therapeutic potency using RNAi as an interventional modality.

### Example 3

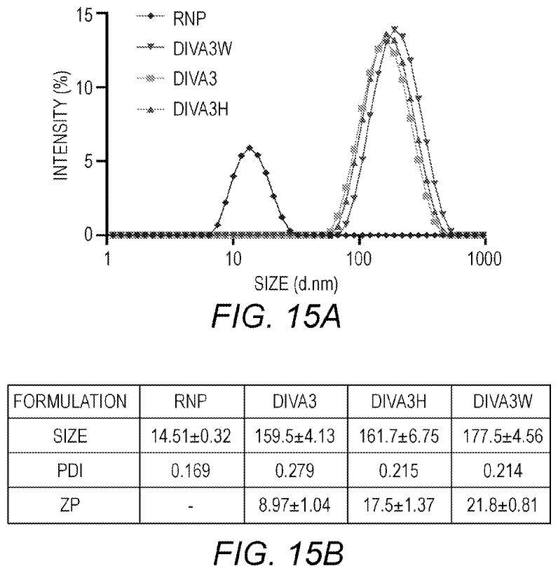

The size analysis and zeta potential of the nanocomplexes was determined using dynamic light scattering at 80:1 molar ratio of peptides relative to ribonucleoprotein (FIGS. 15A-15B), which show the size distribution of the DIVA3W, DIVA3, and DIVA3H peptides (FIG. 15A) and the PDI and zeta potential of the assembled Cas9/gRNA nanocomplexes (FIG. 15B).

Peptide nanocomplexes delivered Cy3-siRNA in MCF10A cells. FIG. 16 shows fluorescence microscopy analysis of the peptides complexed with Cas9/Cy-3 labeled siRNA (red) incubated with MCF10A clover cells (green) for 4 h hours. Nuclei are counterstained with DAPI (blue).

### Example 4

Fusogenic peptides comprising a shorter second peptide sequence were tested. The data show that DIVA3, DIV3H, AND DIV3W peptides containing a penta-arginine tail did not fully complex with siRNAs at various N:P molar ratios (FIG. 17). Shown as white bands, free siRNA was observed for all N:P conditions for each of the three peptide systems. MWM-molecular weight marker.

### Example 5

With nearly 21,000 deaths due to ovarian cancer expected in the U.S. in 2021 and a 5-year survival rate of 27% in stage III and 16% in stage IV diagnoses, there is a significant need for improved treatment strategies and options. The growth and migration rate of epithelial ovarian cancer is the largest contributor to disease severity and late-stage diagnoses, as most patients are not diagnosed until distant metastases are observed, leading to the low survival rates mentioned. Upon diagnosis, aggressive therapeutic approaches are implemented through surgical mass removal and systemic administration of platinum-based chemotherapeutics. A vast majority of these patients experience relapse and progression of disease.

Gene therapy through RNA interference (RNAi) has garnered much attention in recent years and has had several successful applications both in vivo and in clinical models. While this mechanism of cancer therapy is attractive, there are several barriers that complicate efficient delivery of bioactive siRNA: degradation by serum endonucleases and RNases, poor interaction with the cell membrane restricting cellular uptake, and entrapment in the endosomal compartment resulting in lysosomal trafficking and degradation of the siRNA. These issues necessitate development of a robust nanocarrier that can fully complex and protect siRNAs as well as deliver the siRNA cargo into target cells and facilitate endosomal escape into the cytosol. Herein, peptide-based nanocarriers show the ability to deliver RNAi therapeutics to cancer cells in vitro and in vivo. Specifically, fusogenic peptides are used to encourage rapid endosomal membrane destabilization and release of siRNA cargo into the cytosol, allowing for recruitment of RNAi machinery and mRNA destruction. The efficacy of a novel fusogenic peptide, termed DIV3W, is demonstrated herein to deliver siRNAs to ovarian cancer cells in vitro and in vivo, causing significant knockdown in target gene mRNA and protein expression.

The silencing of CSNK2A1 inhibited migration of ovarian cancer cells across a scratch wound and recolonization in comparison to non-targeting siRNA (siNT). Though CSNK2A1 and its protein product, CK2a, is not entirely well understood in ovarian cancer, the in vitro results suggest a significant role in cell-cell communication and increased cell migration, possibly causing high rates of metastasis, recurrence, and aggressiveness in clinical models. Thus, the ability of the DIV3W peptide to efficiently deliver siRNAs in vivo and mediate knockdown of CSNK2A1 gene expression is evaluated herein in a well-characterized xenograft mouse model of ovarian cancer. Two tumor models, intraperitoneal (IP) and subcutaneous (SQ), were established for examination of DIV3W-mediated siRNA biodistribution and bioactivity. SQ tumors were treated via intratumoral (IT) injection and IP tumors were treated via IP administration of peptide-siRNA complexes. The results showed that DIV3W effectively enhanced siRNA localization in ovarian tumor tissue in both models via IP or SQ administration. siRNA uptake was most enhanced via IT administration in SQ tumors, causing significant knockdown of CSNK2A1 mRNA after 48 hours. Further, a multidosing study of DIV3W-siCSNK2A1 in SQ ovarian tumors resulted in 42.6% reduction in tumor volume, further supporting the role of CSNK2A1 in oncogenesis.

### Materials and Methods

Peptide Synthesis and siRNA DIV3W peptide Complexation. (WEADIVADIVWDIVADIVAGGGGG-dRRRRRRRRR) (SEQ ID NO:3) was synthesized to >95% purity at GenScript Inc. (Piscataway, NJ). The DIV3W peptide was complexed with either 1) Cy5.5-labeled siGENOME non-targeting #5 siRNA (siNT-Cy5.5, Dharmacon, Lafayette, CO), 2) non-targeting siRNA, (siNT, Dharmacon), or 3) CSNK2A1-targeted siRNA, (siCSNK2A1, #s3638, ThermoFisher, Waltham, MA). Briefly, DIV3W peptides (10 μM) were incubated for 20 minutes with 100 μM siNT, siNT-Cy5.5, or siCSNK2A1 to a final siRNA dosage of 0.5 mg/kg.

Cell Culture. The epithelial ovarian cancer cell line, OVCAR3, was purchased from the American Type Culture Collection (ATCC, Manassas, VA). OVCAR3 cells were cultured in RPMI 1640 media supplemented with 20% fetal bovine serum (FBS), 1% penicillin/streptomycin, and 0.1% bovine insulin. Cells were maintained at 37° C., 5% CO₂. RPMI 1640 media was replaced with phenol-red free RPMI 1640 media prepared with the same supplements two days prior to harvesting and preparation of cells for animal injections.

Generation of Tumor Xenografts. All mouse procedures and experiments were approved by the Clemson University Institutional Animal Care and Use Committee (IACUC) and completed in accordance with the Guide for the Care and Use of Laboratory Animals (NIH) and Godley-Snell Animal Research Facility (GSRC) Animal Use policies. Procedures for the generation of xenograft subcutaneous and intraperitoneal ovarian cancer tumors were as follows. Athymic nude-FoxN1^{nu} mice were obtained at 6-7 weeks of age (Envigo, Indianapolis, IN). OVCAR3 cells were cultured and grown to 50% confluence, harvested, and 10×10⁶ cells were suspended in 100 μL of 10% Matrigel matrix suspension (Corning, Corning, NY) in 1:1 ratio (v/v) of media/Matrigel. Cell suspensions were injected into mice either SQ on the right flank or IP into the lower right quadrant from the animal's perspective. Tumors were allowed to grow for 14 days before administration of treatment.

In Vivo and Ex Vivo Imaging. After tumor growth was established and tumors measured at least 50 mm³, either siNT-Cy5.5 alone or DIV3W-siNT-Cy5.5 complexes at an siRNA dosage of 0.5 mg/kg were delivered intratumorally to SQ tumors or intraperitoneally to IP tumors. Animals were imaged at 3, 6, 12, and 24 hours post-treatment using the PerkinElmer IVIS Spectrum In Vivo Imaging System (PerkinElmer, Waltham, MA). Gray scale images were obtained using 2×2 binning and fluorescent images were captured using the Cy5 optical filter also using 2×2 binning. Images were acquired and formatted to inverted rainbow spectrum using LivingImage software (PerkinElmer) and exported for fluorescence intensity quantification using Aura software (Spectral Instruments, Tucson, AZ). Following the final 24-hour imaging, animals were euthanized according to IACUC and GSRC protocols. Tumor, heart, lungs, liver, kidneys, and spleen were explanted for ex vivo imaging with quantification as described.

qPCR Analysis. SQ and IP tumors were treated with saline, DIV3W-siNT, or DIV3W-siCSNK2A1 for either 48 hours or in a multidose schedule over a 2-week period. Mice were injected with 0.5 mg/kg of siRNA at each treatment, and multidosing occurred every 3 days for a total of 4 doses. Tumors were measured in two dimensions using digital calipers and tumor volumes calculated using the formula: d²×D/2, where d is the smaller dimension. Mice were euthanized 48 hours post-injection of the final dose. Tumors were resected after euthanasia and stored in RNAlater (Thermo Fisher). Tumors were measured for volume as described above and weighed. Tumor tissue was then homogenized and the lysate was processed using a Qiagen AllPrep RNA/Protein kit (Qiagen, Hilden, Germany). RNA isolation was performed according to Qiagen protocols and was quantified using a Take3 plate (BioTek, Winooksi, VT) and the Gen5 analysis software. RNA was reverse-transcribed using the QuantiTect Reverse Transcription kit (Qiagen). Quantitative real-time PCR was completed with a QuantStudio 3 qPCR system (Applied Biosystems) using Applied Biosystems TaqMan FAST Master Mix (Thermo Fisher) and CSNK2A1 (ID #Hs00751002_s1), AKTI (ID #Hs00178298_m1), VMAC (ID #Hs00418522_m1), CD274 (ID #Hs00204257_m1), and 18S (ID #Hs99999901_s1) predesigned TaqMan probes according to the manufacturer's protocol.

Western Blot Analysis. Tumors were homogenized as previously described. Lysates were processed using the Qiagen AllPrep RNA/Protein kit (Qiagen) and total protein was quantified via bicinchoninic assay according to the manufacturer's protocol. Equivalent protein concentrations for each treatment condition were separated by SDS-PAGE on a BioRad Stain-Free 10% gel according to BioRad protocols and transferred to a Stain-Free PVDF membrane using a wet transfer system. PVDF was cut at appropriate molecular weight intervals consistent with protein weights and blocked for one hour with 5% non-fat dried milk solution in 1× Tris-HCl-buffered-saline-0.1% Tween 20 (TBST) at room temperature. Blots were probed at 4° C. overnight with rabbit monoclonal anti-CK2 antibody (1:1000, Invitrogen) or mouse monoclonal anti-β-actin (1:10,000, Sigma-Aldrich, St. Louis, MO). Membranes were washed five times with 1× TBST and incubated with goat anti-rabbit (1:1000, Thermo Fisher) or goat anti-mouse (1:10,000, Thermo Fisher) secondary antibodies for one hour at room temperature. Five additional washes with 1× TBST were performed and protein bands were detected with SuperSignal West Pico Plus Chemiluminescent Substrate (Thermo Fisher) and imaged using a BioRad ChemiDoc Imaging System (BioRad, Hercules, CA).

Statistical Analysis. Data were acquired to N=3 in biodistribution studies, N=4 in bioactivity analysis, and N=5 in anticancer effects for all experimental groups. Saline controls were complete to N=2 in bioactivity experiments. All data are reported as mean±SEM and comparisons between groups of three or more with one independent variable being analyzed by one-way ANOVA and groups of three or more with more than one independent variable analyzed by two-way ANOVA, all with multiple comparisons between groups using GraphPad Prism 8.4.3.

### Results

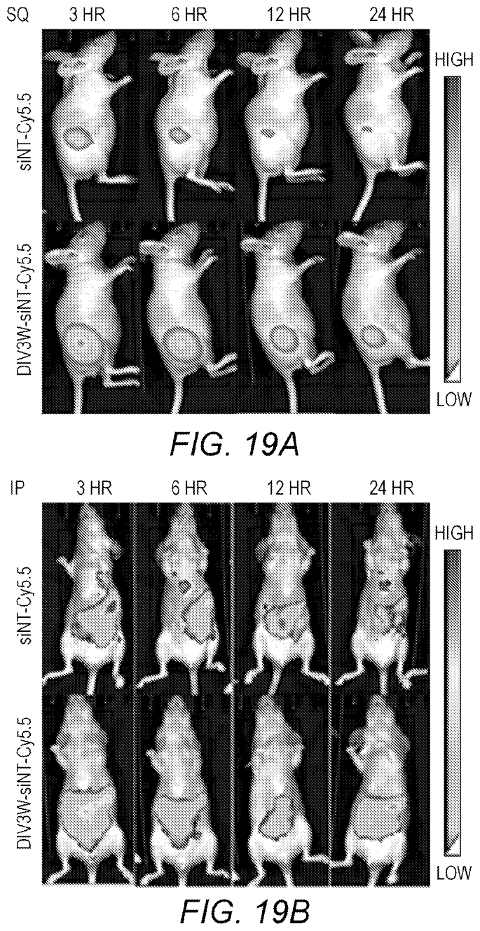

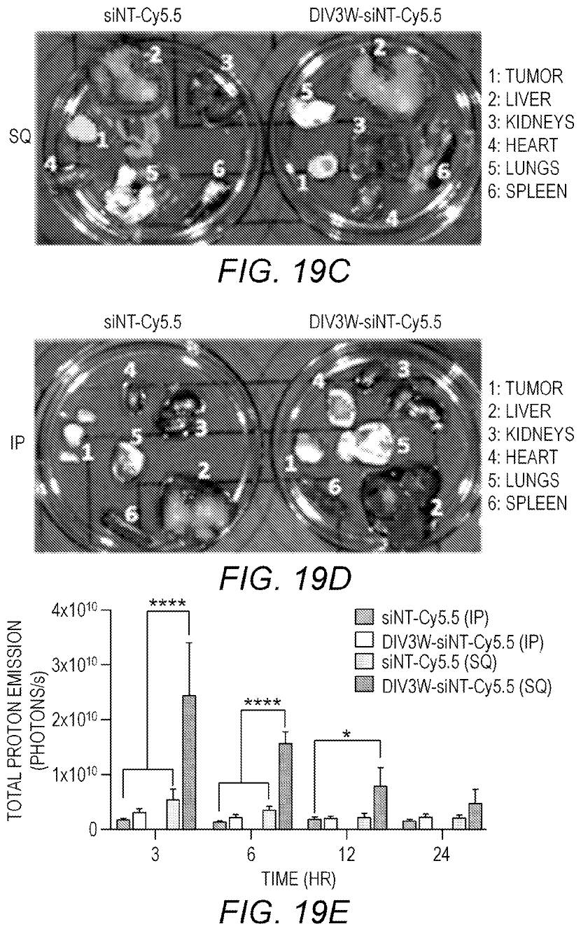

Biodistribution of DIV3W-siRNA Complexes after Intratumoral or Intraperitoneal Administration to IP or SQ Tumors. Mice with subcutaneous and intraperitoneal tumors were treated with either DIV3W-siNT-Cy5.5 or siNT-Cy5.5 alone. SQ tumors were injected intratumorally and IP tumors were treated via intraperitoneal injections. Over the course of 24 hours, DIV3W-mediated delivery of siNT-Cy5.5 exhibited significant tumor localization of fluorescent siNT signal, specifically at 3, 6, and 12-hour post-injection in comparison to siNT-Cy5.5 for all IP treatments (FIGS. 19A, 19B, and 19E). Intratumorally delivered DIV3W-siNT-Cy5.5 complexes exhibited a significantly higher fluorescence intensity over the course of 24 hours, at nearly all time points, in comparison to siNT-Cy5.5 alone. Additionally, these intratumoral injections of DIV3W-siNT-Cy5.5 remained localized to the tumor when compared to the intraperitoneal diffusion in the IP tumor model. Regarding the intraperitoneal delivery, DIV3W-siNT-Cy5.5 also showed higher fluorescence intensity in comparison to siNT-Cy5.5 alone (FIG. 19E). Further, IVIS imaging revealed differences in the localization of fluorescent signal; focal distribution of siRNA over the SQ tumor was observed in intratumoral treatments (FIG. 19A) while regional fluorescence within the entire IP cavity was observed for IP tumors with intraperitoneal treatment (FIG. 19B). Both SQ and IP injection of DIV3W-siNT-Cy5.5 complexes exhibited higher fluorescence across the entire 24-hour period in comparison to siNT-Cy5.5 alone, suggesting increased peptide protection of siRNA cargo. Ex vivo imaging of harvested organs and tumors from the SQ group (FIG. 19C) and IP group (FIG. 19D) revealed higher fluorescence intensity of siNT-Cy5.5 in the tumor tissue in comparison to other tissues. Both SQ and IP tumors exhibited some distribution of siRNA fluorescence in the liver. Low siRNA fluorescence was also observed in the kidneys of mice with SQ tumors and spleen of mice with IP tumors.

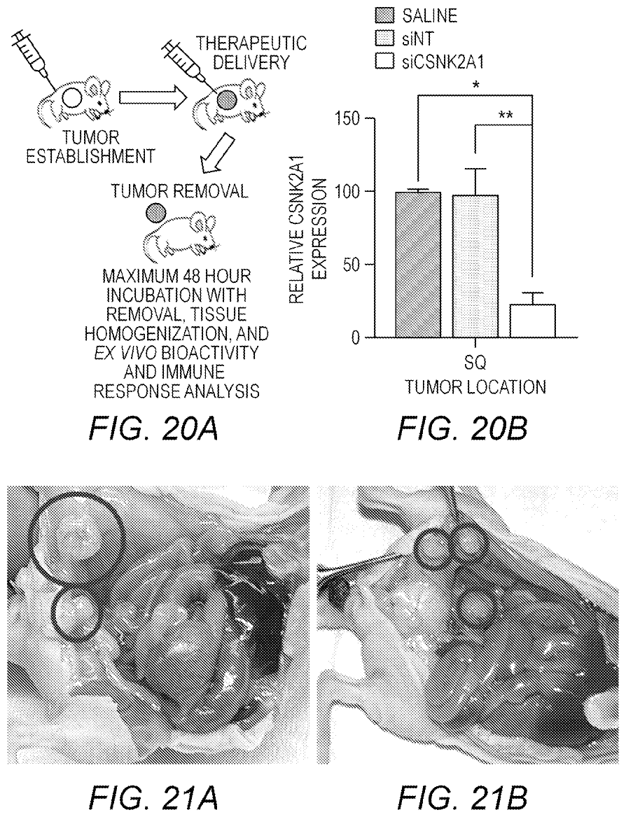

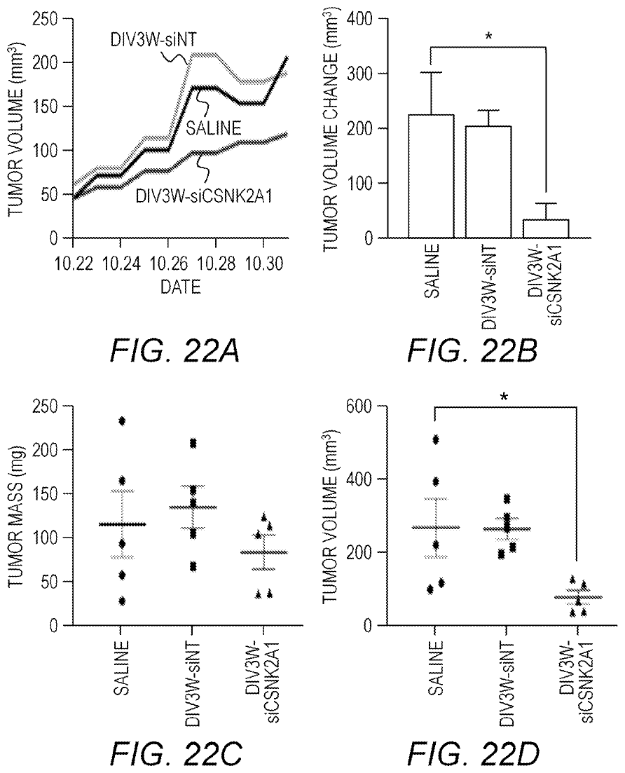

DIV3W-Mediated Delivery of siCSNK2A1 Causes Gene Silencing in SQ Tumors After a Single-Dose Treatment. Bioactivity of siRNAs delivered via DIV3W was evaluated in both SQ and IP tumor models (FIG. 20A) 48 hours post-treatment. In a single dose of 0.5 mg/kg siRNA, DIV3W-siCSNK2A1 efficiently silenced expression of CSNK2A1 mRNA to 22.53%=8.24% when delivered intratumorally to SQ tumors in comparison to saline and DIV3W-siNT controls (FIG. 20B). Also noted were multiple IP metastatic events prior to DIV3W-siRNA treatments (FIGS. 21A-21B). Delivery of OVCAR3 cells to the IP cavity, even with 50% v/v matrigel substrate, were not able to be confined to one location, resulting in spreading throughout the abdomen. Several mice with intraperitoneal metastasis were identified in the DIV3W-siCSNK2A1 group, which may have contributed to the increased CSNK2A1 expression within the IP harvested tumors. Thus, subsequent multidosing treatments were conducted using SQ tumors which successfully demonstrated efficient siRNA delivery and gene knockdown.

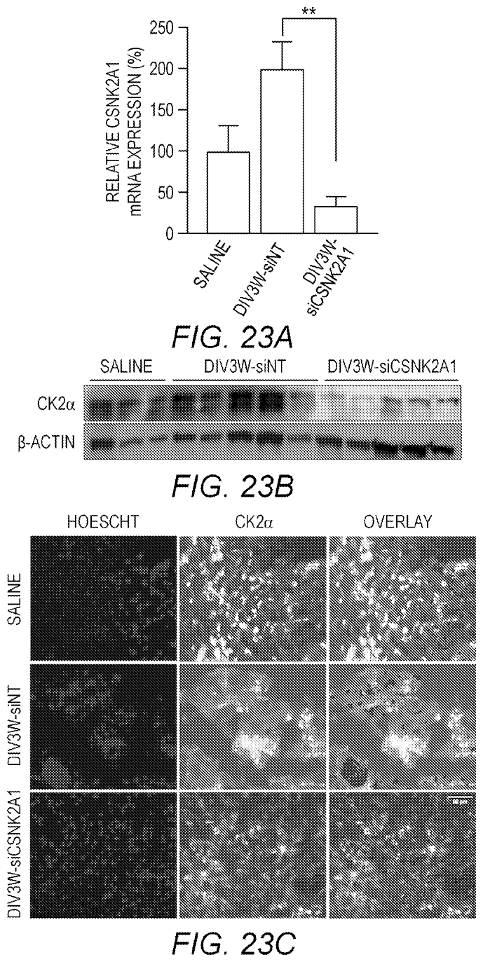

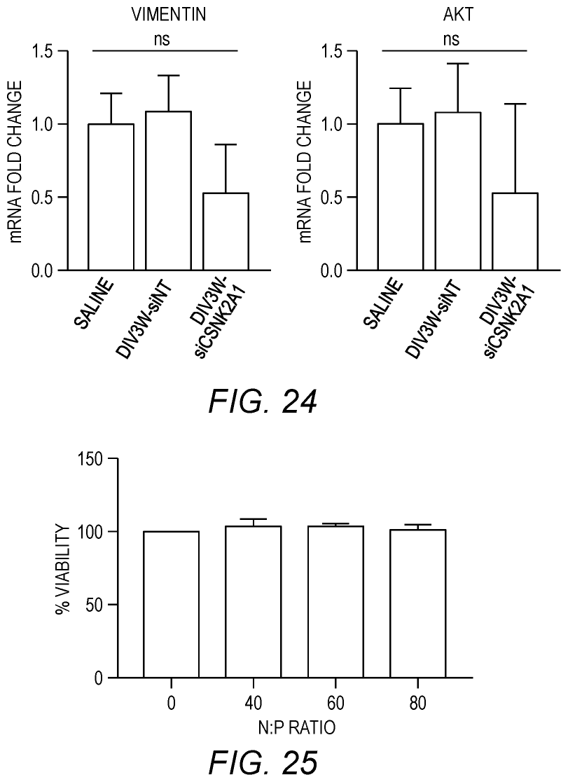

DIV3W-siCSNK2A1-mediated Knockdown of CSNK2A1 Reduces Tumor Burden in SQ Tumors Following a Multidosing Schedule. A multidosing study was completed to evaluate the clinical efficacy of the DIV3W peptide delivery system and CSNK2A1 as a gene target with therapeutic relevance. Mice with SQ flank ovarian tumors were intratumorally injected with saline, DIV3W-siNT, or DIV3W-siCSNK2A1 in four doses over a 2-week period. Excised tumors were evaluated for CSNK2A1 mRNA (FIG. 23A) and CK2α protein (FIG. 23B) expression. qPCR analysis revealed that mice treated with DIV3W-siCSNK2A1 yielded only 37.7%+11.2% CSNK2A1 mRNA expression relative to saline controls and 16.4%+11.2% mRNA expression compared with DIV3W-siNT-treated animals (FIG. 23A). A reduction in CK2α protein production was also noted through western blotting (FIG. 23B) and immunocytochemistry analysis of tumor tissue (FIG. 23C). Five animals in each treatment group were evaluated for changing tumor volume over time (FIG. 22A). Though all groups did have tumor growth, DIV3W-siCSNK2A1 treatments reduced the growth rate such that the final measurement was just 57.41%±14.23% the total volume of the negative control saline treated tumors, with treated tumors only 33.37±26.68 mm³ larger than the volume at the first treatment injection (FIG. 22B). In contrast, DIV3W-siNT-treated tumors were 205.25±25.77 mm³ larger in volume, and saline-treated tumors grew 224.22±78.51 mm³ over the course of the study, where DIV3W-siNT treated animals resulted in 90.92%±7.36% total growth of the saline controls. (FIG. 22B). The mean volumes of resected tumors were 78.29±18.07 mm³, 266.74±27.37 mm³, and 270.67±80.21 mm³ for mice treated with DIV3W-siCSNK2A1, DIV3W-siNT, and saline, respectively. Additionally, vimentin and Akt mRNA levels were assessed to determine downstream effects on cellular migration, growth, and proliferation. In tumors treated with DIV3W-siCSNK2A1, vimentin and Akt mRNA levels were downregulated 2-fold; however, the differences were not statistically significant between the DIV3W-siCSNK2A1 treatment and saline or siNT controls (FIG. 24).

### Discussion and Conclusions

RNAi is a promising option as an alternative or supplementary treatment to chemotherapy in the oncologic clinical space. Several studies have shown the potency of gene knockdown by delivering siRNAs either locally or systemically to tumor tissue, yielding anticancer effects and reduction of tumor growth when compared against saline or non-targeting controls. However, barriers to clinical translation are still prevalent, with many delivery systems not adequately protecting siRNAs during transport and allowing serum degradation and rapid clearance through the reticuloendothelial system. Peptide systems, particularly fusogenic peptides, are shown herein to exhibit the ability to complex and protect siRNAs to overcome some of the hallmark barriers to translation of drug and gene delivery systems. The fusogenic DIV3W peptide is shown herein to mediate delivery of bioactive siRNAs in vitro, causing a significant reduction in targeted mRNA and protein expression. Further, silencing of the target oncogene, CSNK2A1, reduced the ability of ovarian cancer cells to migrate and recolonize after treatment. To further evaluate the clinical translatability of this peptide system as a gene delivery tool, the DIV3W peptide's ability to delivery bioactive siRNAs into ovarian tumor tissue was examined using a xenograft murine model.

The cell line used in this study, OVCAR3, was selected because of its clinical relevance to high-grade serous ovarian adenocarcinoma. However, this cell line has had some difficulty in developing xenograft tumor models without the use of a supplementary basement matrix or delivering high quantities of cells. To address this issue, 10×10⁶ cells OVCAR3 cells in a 50% v/v solution containing 10% Matrigel were delivered. Using this cell dosage, tumors were formed subcutaneously and were easily observed, but IP injected cells exhibited sporadic development of palpable tumors. Additionally, IP delivery of cell/Matrigel suspensions were easily disseminated throughout the IP cavity, and resulted in the growth of multiple tumors with higher likelihood of significant tumor burden.

After establishing the tumor model both through SQ and IP growth, the ability of the DIV3W peptide to deliver fluorescent siRNAs to the tumor environment and remain local to the TME for 24 hours was examined. Following the in vitro results discussed herein, DIV3W-mediated delivery of siNT-Cy5.5 resulted in increased fluorescence in the tumor for both the SQ and IP groupings in comparison to siNT-Cy5.5 alone. Additionally, intratumoral injected SQ tumors exhibited high fluorescence intensity in a focal area, suggesting DIV3W's ability to remain in the TME and protect these injected siRNAs in a physiological environment over time. This protection was also observed in the intraperitoneal delivery route, though fluorescence was not confined to the tumor area. It is important to consider though that the DIV3W peptide in this experiment was not functionalized with an active targeting system, so distribution across the abdominal area was not a surprising result.

Significant fluorescence intensity and localization to the tumor through intratumoral delivery in SQ models caused the selection of this model for bioactivity and anticancer studies, in addition to the metastatic events observed in animals with IP-developed tumors. When treated with DIV3W-siCSNK2A1, SQ tumor homogenate yielded significantly less CSNK2A1 mRNA expression. Compared to saline and DIV3W-siNT controls, DIV3W-siCSNK2A1 groups only showed 22.53%±8.24% gene expression. In addition to tumor retention, the DIV3W peptide is causing cellular uptake and endosomal membrane fusion, releasing bioactive siRNAs for activation of the RNAi pathway. These results suggest a therapeutic potential for the DIV3W delivery system and for CSNK2A1 as a target. However, in order to further support this claim, a multidose schedule of saline, DIV3W-siNT, and DIV3W-siCSNK2A1 was completed to evaluate the downstream effects of tumor growth after gene knockdown.

Anticancer studies were completed through a two-week multidose schedule of saline, DIV3W-siNT, or DIV3W-siCSNK2A1 treatment every 3 days for a total of 4 doses. Tumor volume and mouse mass changes were recorded over the total treatment period and DIV3W-siCSNK2A1 caused a repressed rate of tumor growth compared to saline and DIV3W-siNT controls. Saline and DIV3W-siNT treated animals were reported to grow tumors to 208.7±40.7 mm³ and 189.76±15.36 mm³ respectively, with DIV3W-siCSNK2A1 treated SQ tumors only grew to an average volume of 119.82±29.7 mm³. Translating these measurements to relative total growth, DIV3W-siCSNK2A1 treated tumors only grew to 57.41%±14.23% of saline treated tumors. The significance of tumor growth suppression speaks to the ability of siCSNK2A1 to reduce cell migration. This migration reduction possibly plays a role in reducing the expansion rate of the tumor volume and reduced growth rates observed using DIV3W-siCSNK2A1 in SQ tumors.

This work highlighting the ability of the DIV3W fusogenic peptide to deliver bioactive siRNAs in vivo and suppressing tumor growth provides support to the clinical applicability of the peptide system and gene target CSNK2A1. We have shown the increased tumor retention and fluorescence of siRNA when delivered via DIV3W in comparison to siRNA delivered alone, highlighting the complexation and protection of therapeutic cargo by DIV3W in a physiological environment. Additionally, the significant knockdown of CSNK2A1 in a SQ tumor showing efficient endosomal escape and release of bioactive siRNAs, illustrates the fusogenic capabilities of the DIV3W peptide and role fusogenic peptides play in therapeutic delivery. This gene knockdown caused reduction in tumor growth and reinforces the clinical relevance of CSNK2A1 in ovarian cancer growth. DIV3W has the ability to be a robust delivery vehicle for nucleic acid therapies.

### Example 6

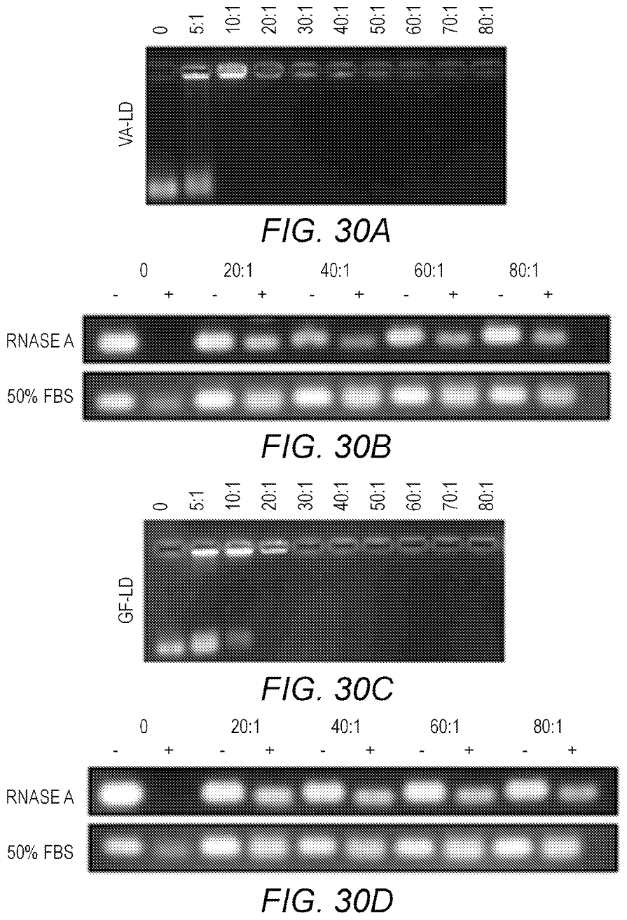

Peptides VA-LD and GF-LD, comprising the cleavable linker VA or GLFG, respectively, between the LHRH target sequence and the DIV3W peptide, and non targeting siRNA were electrostatically complexed at increasing N:P ratios then subjected to agarose gel electrophoresis to determine optimal siRNA binding for each peptide. VA-LD and GF-LD peptides bind siRNA at N:P ratios of 10:1 or higher and protect siRNA in physiologically relevant environments. Peptide-siRNA nanocomplexes at increasing N:P ratios for peptides for VA-LD (FIG. 30A) and GF-LD (FIG. 30C) were incubated in either Rnase A enzyme or in 50% fetal bovine serum (FBS) for 1 h, dissociated using SDS then subjected to gel electrophoresis to visualize siRNA bands. Gels showing increasing N:P ratios of peptide:siRNA for VA-LD (FIG. 30B) and GF-LD (FIG. 30D) from 20:1 to 80:1 N:P; white bands are indicative of intact siRNA

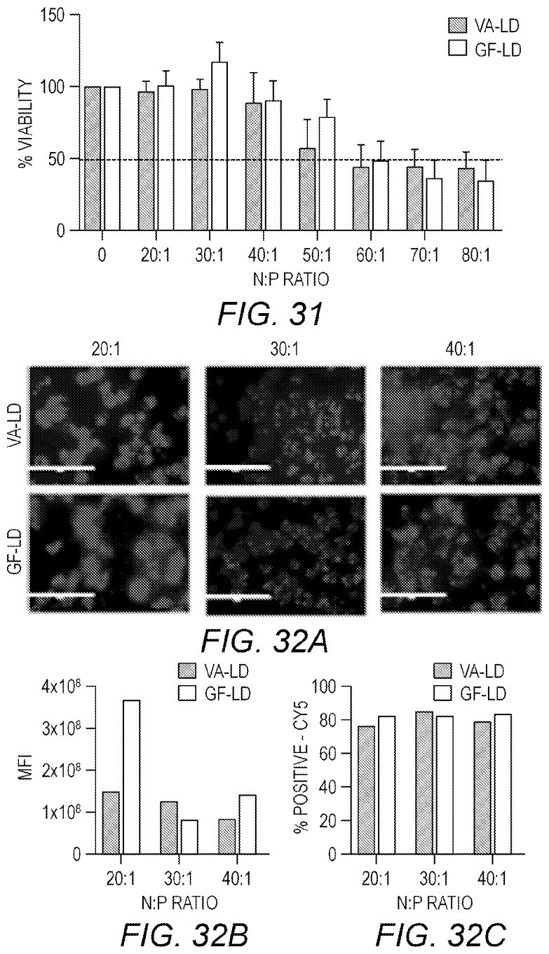

VA-LD and GF-LD peptides were electrostatically complexed with and non targeting siRNA at increasing N:P ratios. Complexes were delivered to human ovarian ES2 cells for 48 hours and analyzed via MTS assay. FIG. 31 confirms VA-LD and GF-LD peptides are biocompatible with ES2 ovarian cancer cells at least at N:P ratio of ≤40:1.

Peptides, VA-LD and GF-LD, and cy5 labeled non targeting siRNA were electrostatically complexed at increasing N:P ratios and delivered to human ovarian ES2 cells for 4 hours. After 4 hours, cells were imaged using fluorescence microscopy then quantified via flow cytometry for mean fluorescence intensity (MFI) and cells positive for siRNA (FIGS. 32A-32C), showing the fusogenic peptides with cleavable linkers deliver siRNA to ES2 ovarian cancer cells at N:P ratios of 20:1-40:1.

All publications, patents, and patent applications are herein incorporated by reference to the same extent as if each individual publication, patent, or patent application was specifically and individually indicated to be incorporated by reference.

Although the foregoing invention has been described in some detail by way of illustration and example for purposes of clarity of understanding, it will be obvious that certain changes and modifications may be practiced within the scope of the list of the foregoing embodiments and the appended claims.

1. A fusogenic peptide for delivery of a cargo to a cell, the fusogenic peptide comprising:
a first peptide sequence with amphipathic properties; and
a second peptide sequence comprising two or more positively-charged amino acid residues for binding the cargo.

2. The fusogenic peptide of claim 1, wherein the first peptide sequence is from 8 to 38 residues in length.

3. The fusogenic peptide of claim 1, wherein the first peptide sequence comprises one or more sequences of DIVX, wherein X is any amino acid.

4. The fusogenic peptide of claim 3, wherein the first peptide sequence comprises from 2 to 6 repeat sequences of DIVX.

5. The fusogenic peptide of claim 3, wherein each X of the sequence of DIVX is independently A, H, or W.

6. The fusogenic peptide of claim 1, wherein the second peptide sequence comprises from 6 to 16 amino acid residues, optionally 9 amino acid residues.

7. (canceled)

8. The fusogenic peptide of claim 1, wherein at least one of the two or more postively-charged amino acids is arginine, optionally D-arginine.

9. (canceled)

10. The fusogenic peptide of claim 1 further comprising a targeting peptide sequence, optionally wherein the targeting peptide targets luteinizing hormone releasing hormone (LHRH) receptor or human epidermal growth factor receptor 2 (HER2).

11. (canceled)

12. The fusogenic peptide of claim 1, further comprising a first linker between the first peptide sequence and the second peptide sequence.

13. The fusogenic peptide of claim 10, further comprising a second linker between the first peptide sequence and the targeting peptide sequence.

14. The fusogenic peptide of claim 13, wherein the first linker and/or the second linker are a glycine linker, each independently comprising 1 to 10 glycine residues.

15. The fusogenic peptide of claim 13, wherein the first linker and/or the second linker is a cleavable linker, optionally wherein the cleavable linker comprises VA or GFLG (SEQ ID NO:7).

16. (canceled)

17. The fusogenic peptide of claim 12, wherein the fusogenic peptide comprises, in order from the N-terminal end, the first peptide, the linker and the second peptide.

18. The fusogenic peptide of claim 13, wherein the fusogenic peptide comprises, in order from the N-terminal end, the targeting peptide, the second linker, the first peptide, the first linker, and the second peptide.

19. The fusogenic peptide of claim 1, comprising the sequence of SEQ ID NO:1, SEQ ID NO:2, SEQ ID NO:3, or a sequence at least 90% identical thereto.

20. The fusogenic peptide of claim 19, wherein one or more amino acid residues are biologically or chemically modified, optionally wherein the modification is glycosylation.

21. (canceled)

22. A nanoparticle complex comprising the fusogenic peptide of claim 1 and a cargo.

23-29. (canceled)

30. A composition comprising the fusogenic peptide of claim 1 and a suitable carrier, diluent, or excipient.

31-34. (canceled)

35. A method of delivering a cargo into a cell, the method comprising contacting the cell with the composition of claim 30, thereby delivering the cargo to the cell.

36-37. (canceled)

38. A method of treating, preventing, or delaying progression of a disease or condition in a subject in need thereof, the method comprising administering to the subject an effective amount of the composition of claim 30, wherein the disease or condition is treatable by the cargo present in the composition.

39-49. (canceled)
